SEQUENCE LISTING (61 sequences)

SEQ ID NO: 1 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 2 - protein, 45 aa, Homo sapiens
TTTPAPRPPTPAPTIASQPLSLRPEACRPAAGGAVHTRGLDFACD

SEQ ID NO: 3 - protein, 45 aa, Artificial Sequence
  Made in Lab - Modified CD8alpha hinge
TTTPAPRPPTPAPTIASQPLSLRPEASRPAAGGAVHTRGLDFACD

SEQ ID NO: 4 - protein, 45 aa, Artificial Sequence
  Made in Lab - Modified CD8alpha hinge
TTTPAPRPPTPAPTIASQPLSLRPEACRPAAGGAVHTRGLDFASD

SEQ ID NO: 5 - protein, 45 aa, Artificial Sequence
  Made in Lab - Modified CD8alpha hinge
TTTPAPRPPTPAPTIASQPLSLRPEASRPAAGGAVHTRGLDFASD

SEQ ID NO: 6 - protein, 21 aa, Homo sapiens
IYIWAPLAGTCGVLLLSLVIT

SEQ ID NO: 7 - protein, 22 aa, Homo sapiens
SGQVLLESNIKVLPTWSTPVQP

SEQ ID NO: 8 - protein, 15 aa, Homo sapiens
KHLCPSPLFPGPSKP

SEQ ID NO: 9 - protein, 36 aa, Homo sapiens
APPRASALPAPPTGSALPDPQTASALPDPPAASALP

SEQ ID NO: 10 - protein, 39 aa, Homo sapiens
DTGLYICKVELMYPPPYYLGIGNGTQIYVIDPEPCPDSD

SEQ ID NO: 11 - protein, 38 aa, Homo sapiens
QIKESLRAELRVTERRAEVPTAHPSPSPRPAGQFQTLV

SEQ ID NO: 12 - protein, 15 aa, Homo sapiens
EPKSCDKTHTCPPCP

SEQ ID NO: 13 - protein, 12 aa, Homo sapiens
ERKCCVECPPCP

SEQ ID NO: 14 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 15 - protein, 12 aa, Homo sapiens
ESKYGPPCPSCP

SEQ ID NO: 16 - protein, 231 aa, Homo sapiens
EPKSCDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 140 aa, Homo sapiens
EPKSCDKTHTCPPCPGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGLQLDETCAEAQDGELDG

SEQ ID NO: 18 - protein, 229 aa, Homo sapiens
ESKYGPPCPSCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 229 aa, Homo sapiens; identical to SEQ ID NO: 18

SEQ ID NO: 20 - protein, 53 aa, Homo sapiens
SGTYLCGAISLAPKAQIKESLRAELRVTERRAEVPTAHPSPSPRPAGQFQTLV

SEQ ID NO: 21 - protein, 5 aa, Artificial Sequence
  Peptide linker sequence
DGGGS

SEQ ID NO: 22 - protein, 5 aa, Artificial Sequence
  Peptide linker sequence
TGEKP

SEQ ID NO: 23 - protein, 4 aa, Artificial Sequence
  Peptide linker sequence
GGRR

SEQ ID NO: 24 - protein, 5 aa, Artificial Sequence
  Peptide linker sequence
GGGGS

SEQ ID NO: 25 - protein, 14 aa, Artificial Sequence
  Peptide linker sequence
EGKSSGSGSESKVD

SEQ ID NO: 26 - protein, 18 aa, Artificial Sequence
  Peptide linker sequence
KESGSVSSEQLAQFRSLD

SEQ ID NO: 27 - protein, 8 aa, Artificial Sequence
  Peptide linker sequence
GGRRGGGS

SEQ ID NO: 28 - protein, 9 aa, Artificial Sequence
  Peptide linker sequence
LRQRDGERP

SEQ ID NO: 29 - protein, 12 aa, Artificial Sequence
  Peptide linker sequence
LRQKDGGGSERP

SEQ ID NO: 30 - protein, 16 aa, Artificial Sequence
  Peptide linker sequence
LRQKDGGGSGGGSERP

SEQ ID NO: 31 - protein, 18 aa, Artificial Sequence
  Peptide linker sequence
GSTSGSGKPGSGEGSTKG

SEQ ID NO: 32 - protein, 7 aa, Artificial Sequence
  Cleavage sequence by TEV protease
  misc_feature (2)..(3): Xaa is any amino acid
  misc_feature (5)..(5): Xaa is any amino acid
  MISC_FEATURE (7)..(7): Xaa = Gly or Ser
EXXYXQX

SEQ ID NO: 33 - protein, 7 aa, Artificial Sequence
  Cleavage sequence by TEV protease
ENLYFQG

SEQ ID NO: 34 - protein, 7 aa, Artificial Sequence
  Cleavage sequence by TEV protease
ENLYFQS

SEQ ID NO: 35 - protein, 22 aa, Artificial Sequence
  Self-cleaving polypeptide comprising 2A site
GSGATNFSLLKQAGDVEENPGP

SEQ ID NO: 36 - protein, 19 aa, Artificial Sequence
  Self-cleaving polypeptide comprising 2A site
ATNFSLLKQAGDVEENPGP

SEQ ID NO: 37 - protein, 14 aa, Artificial Sequence
  Self-cleaving polypeptide comprising 2A site
LLKQAGDVEENPGP

SEQ ID NO: 38 - protein, 21 aa, Artificial Sequence
  Self-cleaving polypeptide comprising 2A site
GSGEGRGSLLTCGDVEENPGP

SEQ ID NO: 39 - protein, 18 aa, Artificial Sequence
  Self-cleaving polypeptide comprising 2A site
EGRGSLLTCGDVEENPGP

SEQ ID NO: 40 - protein, 13 aa, Artificial Sequence
  Self-cleaving polypeptide comprising 2A site
LLTCGDVEENPGP

SEQ ID NO: 41 - protein, 23 aa, Artificial Sequence
  Self-cleaving polypeptide comprising 2A site
GSGQCTNYALLKLAGDVESNPGP

SEQ ID NO: 42 - protein, 20 aa, Artificial Sequence
  Self-cleaving polypeptide comprising 2A site
QCTNYALLKLAGDVESNPGP

SEQ ID NO: 43 - protein, 14 aa, Artificial Sequence
  Self-cleaving polypeptide comprising 2A site
LLKLAGDVESNPGP

SEQ ID NO: 44 - protein, 25 aa, Artificial Sequence
  Self-cleaving polypeptide comprising 2A site
GSGVKQTLNFDLLKLAGDVESNPGP

SEQ ID NO: 45 - protein, 22 aa, Artificial Sequence
  Self-cleaving polypeptide comprising 2A site
VKQTLNFDLLKLAGDVESNPGP

SEQ ID NO: 46 - protein, 14 aa, Artificial Sequence
  Self-cleaving polypeptide comprising 2A site
LLKLAGDVESNPGP

SEQ ID NO: 47 - protein, 19 aa, Artificial Sequence
  Self-cleaving polypeptide comprising 2A site
LLNFDLLKLAGDVESNPGP

SEQ ID NO: 48 - protein, 19 aa, Artificial Sequence
  Self-cleaving polypeptide comprising 2A site
TLNFDLLKLAGDVESNPGP

SEQ ID NO: 49 - protein, 14 aa, Artificial Sequence
  Self-cleaving polypeptide comprising 2A site
LLKLAGDVESNPGP

SEQ ID NO: 50 - protein, 17 aa, Artificial Sequence
  Self-cleaving polypeptide comprising 2A site
NFDLLKLAGDVESNPGP

SEQ ID NO: 51 - protein, 20 aa, Artificial Sequence
  Self-cleaving polypeptide comprising 2A site
QLLNFDLLKLAGDVESNPGP

SEQ ID NO: 52 - protein, 24 aa, Artificial Sequence
  Self-cleaving polypeptide comprising 2A site
APVKQTLNFDLLKLAGDVESNPGP

SEQ ID NO: 53 - protein, 40 aa, Artificial Sequence
  Self-cleaving polypeptide comprising 2A site
VTELLYRMKRAETYCPRPLLAIHPTEARHKQKIVAPVKQT

SEQ ID NO: 54 - protein, 18 aa, Artificial Sequence
  Self-cleaving polypeptide comprising 2A site
LNFDLLKLAGDVESNPGP

SEQ ID NO: 55 - protein, 40 aa, Artificial Sequence
  Self-cleaving polypeptide comprising 2A site
LLAIHPTEARHKQKIVAPVKQTLNFDLLKLAGDVESNPGP

SEQ ID NO: 56 - protein, 33 aa, Artificial Sequence
  Self-cleaving polypeptide comprising 2A site
EARHKQKIVAPVKQTLNFDLLKLAGDVESNPGP

SEQ ID NO: 57 - DNA, 10 bp, Artificial Sequence
  Consensus Kozak sequence
gccrccatgg

SEQ ID NO: 58 - protein, 4 aa, Artificial Sequence
  Exemplary rule for determining light chain CDR-L3 motif
  MOD_RES (3)..(3): Xaa is any amino acid
FGXG

SEQ ID NO: 59 - protein, 4 aa, Artificial Sequence
  Exemplary rule for determining heavy chain CDR-H1 motif
  MOD_RES (2)..(4): Xaa is any amino acid
CXXX

SEQ ID NO: 60 - protein, 5 aa, Artificial Sequence
  Exemplary rule for determining heavy chain CDR-H2 motif
LEWIG

SEQ ID NO: 61 - protein, 4 aa, Artificial Sequence
  Exemplary rule for determining heavy chain CDR-H3 motif
  MOD_RES (3)..(3): Xaa is any amino acd
WGXG

(19) United States
(12) Patent Application Publication
(10) Pub. No.: US 2024/0261404 A1
(43) Pub. Date: Aug. 8, 2024

(54) MODIFIED CCR POLYPEPTIDES AND USES THEREOF

(71) Applicant: 2seventy bio, Inc., Cambridge, MA (US)

(72) Inventor: JOHN W. EVANS, NORTON, MA (US)

(73) Assignee: 2seventy bio, Inc., Cambridge, MA (US)

(21) Appl. No.: 17/918,830

(22) PCT Filed: Apr. 16, 2021
(86) PCT No.: PCT/US2021/027648

§ 371 Date: Oct. 13, 2022

Related U.S. Application Data
(60) Provisional application No. 63/011,494, filed on Apr. 17, 2020.

(51) Int. Cl. A61K 39/00; A61P 35/00; C07K 14/705; C07K 14/725; C12N 5/0783; C12N 15/86

(52) U.S. Cl. CPC A61K 39/4631; A61K 39/4611; A61K 39/4613; A61K 39/4614; A61P 35/00; C07K 14/7051; C07K 14/70517; C12N 5/0636; C12N 15/86; C12N 2510/00; C12N 2740/15043

(57) ABSTRACT

The present disclosure provides improved chimeric co-stimulatory receptors (CCRs), fusion proteins, genetically modified immune effector cells, and use of these compositions to treat disease.

### CROSS REFERENCE TO RELATED APPLICATIONS

This application is a National Stage application under 35 U.S.C. § 371 of International Application No. PCT/US2021/027648, filed Apr. 16, 2021, which claims the benefit under 35 U.S.C. § 119(e) of U.S. Provisional Application No. 63/011,494, filed Apr. 17, 2020, each of which are incorporated by reference herein in their entireties.

### STATEMENT REGARDING SEQUENCE LISTING

The Sequence Listing associated with this application is provided in text format in lieu of a paper copy and is hereby incorporated by reference into the specification. The name of the text file containing the Sequence Listing is BLUE-127_PC_ST25.txt. The text file is 27 KB, created on Apr. 14, 2021, and is being submitted electronically via EFS-Web, concurrent with the filing of the specification.

### BACKGROUND

### Technical Field

The present disclosure relates to improved compositions and methods for treating disease. More particularly, the disclosure relates to improved chimeric co-stimulatory receptors (CCRs), fusion proteins, genetically modified immune effector cells, and use of these compositions to treat disease.

### Description of the Related Art

Chimeric antigen receptors (CARs) are receptor proteins that combine both antigen-recognition and T-cell activation into a single receptor. CARs are complex proteins having several protein domains, such as an antigen-specific binding domain, a hinge or spacer sequence, a transmembrane domain, one or more co-stimulatory domains, and a signaling domain. T cells engineered to express CARs can be redirected to particular cell types (e.g., cancer cells) expressing a target antigen. Indeed, such CAR T cells have proven useful for inducing T-cell mediated killing of certain leukemias and lymphomas which express the CD19 antigen.

However, despite the excitement around CAR T technology, designing effective CARs continues to be a challenging and a time-consuming endeavor (see e.g., Guedan et al., Mol Ther Methods Clin Dev. 2019 Mar 15; 12: 145-156) and attempts to use CAR T cells have been met with limited success. For example, one major obstacle that limits the efficacy of CAR T cell therapy is relapse of “antigen negative” cancers. Additionally, administration of CAR T cells is also known to cause adverse side effects, e.g., cytokine storms and/or neurotoxicity. Moreover, the therapeutic efficacy of a given antigen binding domain used in a CAR can be unpredictable: if the antigen binding domain binds too strongly, the CAR T cells may induce massive cytokine release resulting in a potentially fatal immune reaction deemed a “cytokine storm,” and if the antigen binding domain binds too weakly, the CAR T cells may not display sufficient therapeutic efficacy in clearing cancer cells. Heterogenicity of antigen expression within tumors also affects the efficacy of CAR T cell therapies.

Successful strategies to improve and/or augment CAR T cell signaling have yet to be realized and represent a substantial impediment to advancing CAR T therapies to a variety of cancers.

### BRIEF SUMMARY

The present disclosure generally relates, in part, to improved CCR polypeptides, fusion proteins, and methods of using the same. Particularly, the invention provides fusion polypeptides comprising a CAR and a modified CCR polypeptide and their use in treating, preventing, or ameliorating of at least one symptom of cancer. More particularly, the CCR comprises a modified hinge region, wherein the CAR/CCR fusion polypeptide displays reduced antigen-independent signaling, e.g., signaling in the absence of a CAR antigen.

In various embodiments, a fusion polypeptide is provided comprising a) a chimeric antigen receptor (CAR) comprising a first hinge region; b) a polypeptide cleavage signal; and c) a chimeric co-stimulatory receptor (CCR) comprising a second hinge region modified to reduce CCR antigen-mediated stimulation of T cell signaling in the absence of CAR antigen compared to a CCR comprising a non-modified hinge region.

In various embodiments, a fusion polypeptide is provided comprising a) a chimeric antigen receptor (CAR) comprising a first hinge region; b) a polypeptide cleavage signal; and c) a chimeric co-stimulatory receptor (CCR) comprising a second hinge region comprising one or more cysteines substituted with another amino acid.

In particular embodiments, the one or more cysteine substitutions within the second hinge region reduces CCR antigen-mediated stimulation of T cell signaling in the absence of CAR antigen, compared to a CCR comprising a non-modified second hinge region.

In particular embodiments, the one or more cysteine substitutions within the second hinge region reduces CAR/CCR association in the absence of CAR antigen, compared to a CCR comprising a non-modified second hinge region.

In various embodiments, the first hinge region is modified, wherein, optionally, the modification comprises one or more substitutions or deletions. In some embodiments, the modified first hinge region comprises one or more cysteines substituted with a different amino acid. In some embodiments, the modified first hinge region reduces CCR antigen-mediated stimulation of T cell signaling in the absence of CAR antigen compared to a CAR comprising a non-modified first hinge region. In some embodiments, the modified first hinge region reduces CAR/CCR association in the absence of CAR antigen compared to a CAR comprising a non-modified first hinge region.

In various embodiments, the one or more cysteine residues within the first and/or second hinge regions are substituted with serine(s) or alanine(s). In some embodiments, the first and second hinge regions are derived from the same protein. In some embodiments, the first and second hinge regions are derived from different proteins.

In various embodiments, the first and/or second hinge region comprises a hinge region, or functional fragment thereof, selected from the group consisting of: a CD8α hinge, a CD4 hinge, a CD28 hinge, a CD7 hinge, a CD152 hinge, a PD-1 hinge, a IgG1 hinge, a IgG2 hinge, a IgG3 hinge, a IgG4 hinge, an IgG1 hinge/CH2/CH3, an IgG1 hinge-CH3-hinge-M1, an IgG1 hinge/CH2/CH3, an IgG1 hinge/CH3/hinge/M1, an IgG4 hinge/CH2/CH3, and an IgG4 hinge/CH2. In particular embodiments, the first and/or second hinge region is a CD8α hinge region, or functional fragment thereof.

In some embodiments, the CD8α hinge region comprises an amino acid substitution at position 27 of SEQ ID NO: 2. In some embodiments, the CD8α hinge region comprises an amino acid substitution at position 44 of SEQ ID NO: 2. In some embodiments, the CD8α hinge region comprises amino acid substitutions at positions 27 and 44 of SEQ ID NO: 2.

In particular embodiments, the first and/or second hinge region comprises an amino acid sequence as set forth in SEQ ID NO: 3. In some embodiments, the first and/or second hinge region comprises an amino acid sequence having at least 90%, at least 93%, at least 95%, or at least 97% identity to the sequence set forth in SEQ ID NO: 3.

In particular embodiments, the first and/or second hinge region comprises an amino acid sequence as set forth in SEQ ID NO: 4. In some embodiments, the first and/or second hinge region comprises an amino acid sequence having at least 90%, at least 93%, at least 95%, or at least 97% identity to the sequence set forth in SEQ ID NO: 4.

In particular embodiments, the first and/or second hinge region comprises an amino acid sequence as set forth in SEQ ID NO: 5. In some embodiments, the first and/or second hinge region comprises an amino acid sequence having at least 90%, at least 93%, at least 95%, or at least 97% identity to the sequence set forth in SEQ ID NO: 5.

In some embodiments, the CD4 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 7, or functional fragment thereof. In some embodiments, the CD28 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 8, or functional fragment thereof. In some embodiments, the CD7 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 9, or functional fragment thereof. In some embodiments, the CD152 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 10, or functional fragment thereof. In some embodiments, the PD-1 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 11, or functional fragment thereof. In some embodiments, the IgG1 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 12, or functional fragment thereof. In some embodiments, the IgG2 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 13, or functional fragment thereof. In some embodiments, the IgG3 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 14, or functional fragment thereof. In some embodiments, the IgG4 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 15, or functional fragment thereof. In some embodiments, the IgG1 hinge/CH2/CH3 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 16, or functional fragment thereof. In some embodiments, the IgG1 Hinge-CH3-Hinge-M1 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 17, or functional fragment thereof. In some embodiments, the IgG4 hinge/CH2/CH3 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 18, or functional fragment thereof. In some embodiments, the IgG4 hinge/CH2 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 19, or functional fragment thereof. In some embodiments, the PD-1 hinge region comprising at least 85%, 86%, 87%, 88%, 89%, 90%, 91%, 92%, 93%, 94%, 95%, 96%, 97%, 98%, 99%, or 100% amino acid identity the amino acid sequence set forth in SEQ ID NO: 20, or functional fragment thereof.

In various embodiments, the CAR comprises a first antibody or antigen-specific binding fragment thereof; and the CCR comprises a second antibody or antigen-specific binding fragment thereof. In some embodiments, the first and/or second antibody or antigen-specific binding fragment thereof is selected from the group consisting of: a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a VHH, a minibody, a diabody, a triabody, a tetrabody, a disulfide stabilized Fv protein (“dsFv”), a single-domain antibody (sdAb, Nanobody), and a camelid antibody (VHH) or fragment thereof. In some embodiments, the first and/or second antibody or antigen-specific binding fragment thereof is an scFv or a VHH.

In some embodiments, the first and second antibody or antigen-specific binding fragments bind the same target antigen. In some embodiments, the first and second antibody or antigen-specific binding fragments bind different epitopes on the same target antigen. In some embodiments, the first and second antibody or antigen-specific binding fragments bind different target antigens.

In various embodiments, the first and/or second antibody or antigen-specific binding fragment binds a target antigen selected from the group consisting of: alpha folate receptor (FRα), αvβ6 integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

In various embodiments, the first antibody or antigen-specific binding fragment binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the second antibody or antigen-specific binding fragment binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands.

In various embodiments, the first antibody or antigen-specific binding fragment binds a target antigen selected from the group consisting of: B cell maturation antigen (BCMA), CD33, CD79a, CD79b, C-type lectin-like molecule-1 (CLL-1), MAGE-A4, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the second antibody or antigen-specific binding fragment binds a target antigen selected from the group consisting of: B cell maturation antigen (BCMA), CD20, epidermal growth factor receptor (EGFR), epidermal growth factor receptor variant III (EGFRvIII), and TAG72.

In various embodiments, the first antibody or antigen-specific binding fragment binds B cell maturation antigen (BCMA); and the second antibody or antigen-specific binding fragment binds a target antigen selected from the group consisting of: epidermal growth factor receptor (EGFR), epidermal growth factor receptor variant III (EGFRvIII), and TAG72.

In various embodiments, the CAR further comprises a first transmembrane domain; and the CCR further comprises a second transmembrane domain. In some embodiments, the first and/or second transmembrane domain is derived from a polypeptide selected from the group consisting of: alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, and PD1. In some embodiments, the first and/or second transmembrane domain is derived from CD8α.

In various embodiments, the CAR further comprises a first intracellular co-stimulatory domain; and the CCR further comprises a second intracellular co-stimulatory domain. In some embodiments, the first and/or second co-stimulatory domain is derived from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, tumor necrosis factor receptor 2 (TNFR2), and ZAP70. In some embodiments, the first and/or second co-stimulatory domain is derived from CD137 (4-1BB). In some embodiments, the first and/or second co-stimulatory domain is derived from CD28.

In various embodiments, the CAR further comprises a primary signaling domain. In some embodiments, the primary signaling domain is derived from a polypeptide selected from the group consisting of: FcRγ, FcRβ, CD3γ, CD3δ, CD3ε, CD3ζ, CD22, CD79a, CD79b, and CD66d. In some embodiments, the primary signaling domain is derived from CD3ζ.

In various embodiments, the CAR comprises a first antibody or antigen-specific binding fragment, a first hinge region, a first transmembrane domain, a first intracellular co-stimulatory domain, and a primary signaling domain; and the CCR comprises a second antibody or antigen-specific binding fragment, a second hinge region, a second transmembrane domain, and a second intracellular co-stimulatory domain.

In particular embodiments, a fusion polypeptide is provided comprising: a) a chimeric antigen receptor (CAR) comprising a first antibody or antigen-specific binding fragment, a first hinge region, a first transmembrane domain, a first intracellular co-stimulatory domain, and a primary signaling domain; b) a polypeptide cleavage signal; and c) a chimeric co-stimulatory receptor (CCR) comprising a second antibody or antigen-specific binding fragment, a second hinge region, a second transmembrane domain, and a second intracellular co-stimulatory domain; wherein the second hinge region comprises one or more cysteine substitutions or deletions that i) reduce CCR antigen-mediated stimulation of T cell signaling in the absence of CAR antigen, compared to a CCR comprising a non-modified second hinge region; or ii) reduce CAR/CCR association in the absence of CAR antigen, compared to a CCR comprising a non-modified second hinge region.

In particular embodiments, a fusion polypeptide is provided comprising a) a CAR comprising a first antibody or antigen-specific binding fragment that bind BCMA, a first hinge region derived from CD8α, a first transmembrane domain derived from CD8α, a first intracellular co-stimulatory domain derived from 4-1BB, and a primary signaling domain derived from CD3ζ; b) a polypeptide cleavage signal; and c) a CCR comprising a second antibody or antigen-specific binding fragment that bind EGFR, a second hinge region derived from CD8α, a second transmembrane domain derived from CD8α, and a second intracellular co-stimulatory domain derived from CD28; wherein the second hinge region comprises one or more cysteine substitutions or deletions that i) reduce CCR antigen-mediated stimulation of T cell signaling in the absence of CAR antigen, compared to a CCR comprising a non-modified second hinge region; or ii) reduce CAR/CCR association in the absence of CAR antigen, compared to a CCR comprising a non-modified second hinge region.

In various embodiments, the modified second hinge region comprises an amino acid sequence selected from the group consisting of: SEQ ID NO: 3, SEQ ID NO: 4, and SEQ ID NO: 5.

In various embodiments, the peptide cleavage signal is a viral self-cleaving polypeptide. In some embodiments, the polypeptide cleavage signal is a viral self-cleaving 2A polypeptide. In some embodiments, the polypeptide cleavage signal is a viral self-cleaving polypeptide selected from the group consisting of: a foot-and-mouth disease virus (FMDV) 2A (F2A) peptide, an equine rhinitis A virus (ERAV) 2A (E2A) peptide, a Thosca asigna virus (TaV) 2A (T2A) peptide, a porcine teschovirus-1 (PTV-1) 2A (P2A) peptide, a Theilovirus 2A peptide, and an encephalomyocarditis virus 2A peptide.

In particular embodiments, a polynucleotide encoding the fusion polypeptide of any one of the fusion polypeptides described herein is provided. In particular embodiments, a vector comprising the polynucleotide is provided. In some embodiments, the vector is an expression vector. In some embodiments, the vector is an episomal vector. In some embodiments, the vector is a viral vector. In some embodiments, the vector is a retroviral vector. In some embodiments, the vector is a lentiviral vector.

In particular embodiments, a cell that expresses a fusion polypeptide describe herein is provided. In some embodiments, the cell comprises a polynucleotide or the vector described herein. In some embodiments, the cell is a genetically engineered host cell. In some embodiments, the cell is a hematopoietic cell. In some embodiments, the cell is a hematopoietic stem or progenitor cell. In some embodiments, the cell is a CD34+ hematopoietic stem or progenitor cell. In some embodiments, the cell is an immune effector cell. In some embodiments, the cell is a T-cell. In some embodiments, the cell is a CD3+, CD4+, and/or CD8+ cell. In some embodiments, the cell is a cytotoxic T lymphocytes (CTLs), a tumor infiltrating lymphocytes (TILs), or a helper T cell. In some embodiments, the T-cell is a αβ-T cell. In some embodiments, the T-cell is a γδ-T cell. In some embodiments, the host cell is a natural killer (NK) cell. In some embodiments, the natural killer cell is a natural killer T (NKT) cell. In some embodiments, the host cell is a macrophage.

In particular embodiments, a composition comprising a cell described herein and a pharmaceutically acceptable carrier is provided.

In particular embodiments, a method of treating a cancer in a subject in need thereof is provided. In various embodiments, the method comprises administering to the subject a therapeutically effective amount of a composition described herein.

In particular embodiments, a method for ameliorating at one or more symptoms associated with a cancer in a subject is provided. In various embodiments, the method comprises administering to the subject a therapeutically effective amount of a composition described herein sufficient to ameliorate at least one symptom associated with the cancer. In some embodiments, the one or more symptoms ameliorated are selected from the group consisting of: weakness, fatigue, shortness of breath, easy bruising and bleeding, frequent infections, enlarged lymph nodes, distended or painful abdomen, bone or joint pain, fractures, unplanned weight loss, poor appetite, night sweats, persistent mild fever, and decreased urination.

In various embodiments, the cancer is solid cancer. In some embodiments, the cancer is a liquid cancer. In some embodiments, the cancer is a hematological malignancy. In some embodiments, the cancer is non-Hodgkin's lymphoma, acute lymphocytic leukemia (ALL), chronic lymphocytic leukemia (CLL), hairy cell leukemia (HCL), multiple myeloma (MM), acute myeloid leukemia (AML), or chronic myeloid leukemia (CML). In some embodiments, the non-Hodgkin's lymphoma is Burkitt's lymphoma, small lymphocytic lymphoma (SLL), diffuse large B cell lymphoma (DLBCL), follicular lymphoma (FL), mantle cell lymphoma (MCL), or marginal zone lymphoma (MZL). In some embodiments, the non-Hodgkin's lymphoma is diffuse large B cell lymphoma (DLBCL). In some embodiments, the cancer is a MM selected from the group consisting of: overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In particular embodiments, a method of generating a population of cells that expresses a fusion polypeptide described herein is provided. In various embodiments, the method comprises introducing into the population of cells a polynucleotide or the vector described herein. In some embodiments, the population of cells comprises hematopoietic stem or progenitor cells. In some embodiments, the population of cells comprises CD34+ hematopoietic stem or progenitor cells. In some embodiments, the population of cells comprises immune effector cells. In some embodiments, the population of cells comprises T cells, NK cells, and/or NKT cells. In some embodiments, the population of cells comprises T cells.

In particular embodiments, a method of reducing CCR co-stimulation of T cell signaling in cells expressing both a CAR and a CCR is provided. In various embodiments, the method comprises a) obtaining CAR and CCR polypeptides each having hinge domain, optionally wherein the CAR and CCR are expressed as a fusion polypeptide; b) substituting one or more cysteine residues within the CCR hinge domain for another residue, thereby producing a modified CCR; and c) expressing the CAR and modified CCR in a cell.

In particular embodiments, a method of reducing CCR antigen-mediated stimulation of T cell signaling in the absence of CAR antigen, compared to a CCR comprising a non-modified hinge, is provided. In various embodiments, the method comprises a) obtaining CAR and CCR polypeptides each having hinge domain, optionally wherein the CAR and CCR are expressed as a fusion polypeptide; b) substituting one or more cysteine residues within the CCR hinge domain for another residue, thereby producing a modified CCR; and c) expressing the CAR and modified CCR in a cell.

In various embodiments, the cysteine residue substitution(s) within the hinge domain of the CCR antigen-mediated stimulation of T cell signaling in the absence of CAR antigen, compared to a CCR comprising a non-modified hinge region. In some embodiments, the cysteine residue substitution(s) within the hinge domain of the CCR reduce(s) association with the CAR in the absence of CAR antigen, compared to a CCR comprising a non-modified hinge region. In some embodiments, both the CCR and CAR comprise modified hinge regions. In some embodiments, the modified CAR hinge region comprises one or more cysteines substituted with a different amino acid. In some embodiments, the one or more cysteines substituted within the CAR hinge region reduces antigen-independent signaling of the CAR, compared to a CAR comprising a non-modified hinge region. In some embodiments, the one or more cysteines substitutes within the CAR hinge region reduces antigen-independent association with the CCR, compared to a CAR comprising a non-modified hinge region. In some embodiments, the CAR and CCR hinge regions are derived from the same protein. In some embodiments, the CAR and CCR hinge regions are derived from different proteins. In some embodiments, the one or more cysteine residues within the CCR and/or CAR hinge regions are substituted with serine(s) or alanine(s).

In various embodiments, the CCR and/or CAR hinge region comprises a hinge region, or functional fragment thereof, selected from the group consisting of: a CD8α hinge, a CD4 hinge, a CD28 hinge, a CD7 hinge, a CD152 (CTLA-4) hinge, a PD-1 hinge, a IgG1 hinge, a IgG2 hinge, a IgG3 hinge, a IgG4 hinge, an IgG1 hinge/CH2/CH3, an IgG1 hinge/CH3/hinge/M1, an IgG4 hinge/CH2/CH3, and an IgG4 hinge/CH2. In some embodiments, the CCR and/or CAR hinge region is a CD8α hinge region, or fragment thereof. In some embodiments, the CCR and/or CAR hinge region is a CD8α hinge region. In some embodiments, the CD8α hinge region of the CCR and/or CAR comprises an amino acid substitution at position 27 of SEQ ID NO: 2. In some embodiments, the CD8α hinge region of the CCR and/or CAR comprises an amino acid substitution at position 44 of SEQ ID NO: 2. In some embodiments, the CD8α hinge region of the CCR and/or CAR comprises amino acid substitutions at positions 27 and 44 of SEQ ID NO: 2. In some embodiments, the CD8α hinge region of the CCR and/or CAR comprises an amino acid sequence as set forth in SEQ ID NO: 3. In some embodiments, the CD8α hinge region of the CCR and/or CAR comprises an amino acid sequence having at least 90%, at least 93%, at least 95%, or at least 97% identity to the sequence set forth in SEQ ID NO: 3. In some embodiments, the CD8α hinge region of the CCR and/or CAR comprises an amino acid sequence as set forth in SEQ ID NO: 4. In some embodiments, the CD8α hinge region of the CCR and/or CAR comprises an amino acid sequence having at least 90%, at least 93%, at least 95%, or at least 97% identity to the sequence set forth in SEQ ID NO: 4. In some embodiments, the CD8α hinge region of the CCR and/or CAR comprises an amino acid sequence as set forth in SEQ ID NO: 5. In some embodiments, the CD8α hinge region of the CCR and/or CAR comprises an amino acid sequence having at least 90%, at least 93%, at least 95%, or at least 97% identity to the sequence set forth in SEQ ID NO: 5.

In various embodiments, the CAR and CCR are expressed as any one of the fusion polypeptides described herein. In various embodiments, step (c) comprises introducing into the cell a polynucleotide or vector encoding the CAR and/or CCR.

### BRIEF DESCRIPTION OF SEVERAL VIEWS OF THE DRAWINGS

FIG. 1A shows IFNγ levels from untransduced T cells (UTD) and CAR T cells expressing an anti-BCMA CAR with and without an anti-EGFR CCR, in the absence or presence of EGFR+ HT-1080 fibrosarcoma cells.

FIG. 1B shows impedance data to calculate % cytotoxicity induced by UTD T cells and CAR T cells expressing an anti-BCMA CAR with and without an anti-EGFR CCR co-cultured with EGFR+ HT-1080 fibrosarcoma cells at Effector:Target (E:T) ratios of 20:1, 10:1, and 5:1.

FIG. 2 shows human A549 tumor volume in immunocompromised NSG mice after treatment with Vehicle, UTD T cells, and anti-BCMA CAR T cells with and without an anti-EGFR CCR.

FIG. 3 shows a cartoon representing several mechanisms by which CCR activation may induce CAR-mediated signaling in the absence of the CAR antigen.

FIG. 4A shows a cartoon representing different CAR/CCR construct pairs having mutations in their hinge regions and a graph showing IFNγ levels from UTD T cells and CAR T cells expressing different CAR/CCR pairs in two different cancer cell lines which express EGFR, with and without exogenous expression of BCMA.

FIG. 4B shows TNFα levels from UTD T cells and CAR T cells expressing different CAR/CCR pairs in two different cancer cell lines which express EGFR, with and without exogenous expression of BCMA.

FIG. 4C shows IL-2 levels from UTD T cells and CAR T cells expressing different CAR/CCR pairs in two different cancer cell lines which express EGFR, with and without exogenous expression of BCMA.

FIG. 5A shows the domains of exemplary CAR/CCR constructs.

FIG. 5B shows FACS expression of CAR and CCR constructs on T cells.

FIG. 6 shows IFNγ levels from UTD T cells and CAR T cells expressing various combinations of anti-BCMA CARs and anti-EGFR CCRs, with or without the hinge C to S mutations, in two cancer cell lines which express EGFR, but not BCMA.

FIG. 7A shows IFNγ levels from CAR T cells expressing various combinations of anti-BCMA CARs and anti-EGFR CCRs, with or without the hinge C to S mutations, in A549 cells (EGFR+) and A549 cells (EGFR+) modified to also express BCMA.

FIG. 7B shows IL-2 levels from CAR T cells expressing various combinations of anti-BCMA CARs and anti-EGFR CCRs, with or without the hinge C to S mutations, in A549 cells (EGFR+) and A549 cells (EGFR+) modified to also express BCMA.

FIG. 7C shows IFNγ levels from CAR T cells expressing various combinations of anti-BCMA CARs and anti-EGFR CCRs, with or without the hinge C to S mutations, in HT-1080 cells (EGFR+) and HT-1080 cells (EGFR+) modified to also express BCMA.

FIG. 7D shows IL-2 levels from CAR T cells expressing various combinations of anti-BCMA CARs and anti-EGFR CCRs, with or without the hinge C to S mutations, in HT-1080 cells (EGFR+) and HT-1080 cells (EGFR+) modified to also express BCMA.

FIG. 8A shows impedance data to calculate % cytotoxicity induced by UTD T cells and CAR T cells expressing various combinations of anti-BCMA CARs and anti-EGFR CCRs, with or without the hinge C to S mutations, in EGFR+ HT-1080 fibrosarcoma cells.

FIG. 8B shows impedance data to calculate % cytotoxicity induced by UTD T cells and CAR T cells expressing various combinations of anti-BCMA CARs and anti-EGFR CCRs, with or without the hinge C to S mutations, in EGFR+ HT-1080 fibrosarcoma cells also modified to express BCMA.

FIG. 8C shows impedance data to calculate % cytotoxicity induced by UTD T cells and CAR T cells expressing various combinations of anti-BCMA CARs and anti-EGFR CCRs, with or without the hinge C to S mutations, in EGFR+ HT-1080 fibrosarcoma cells.

FIG. 8D shows impedance data to calculate % cytotoxicity induced by UTD T cells and CAR T cells expressing various combinations of anti-BCMA CARs and anti-EGFR CCRs, with or without the hinge C to S mutations, in EGFR+ HT-1080 fibrosarcoma cells also modified to express BCMA.

### BRIEF DESCRIPTION OF THE SEQUENCE IDENTIFIERS

SEQ ID NO: 1 sets forth the amino acid sequence of the human CD8α protein (Swiss-Prot accession number P01732).

SEQ ID NO: 2 sets forth the amino acid of an illustrative wildtype CD8α hinge region.

SEQ ID NO: 3 sets forth the amino acid sequence of an illustrative CD8α hinge comprising a cysteine to serine mutation at position 27 of (C27S).

SEQ ID NO: 4 sets forth the amino acid sequence of an illustrative CD8α hinge comprising a cysteine to serine mutation at position 44 (C44S).

SEQ ID NO: 5 sets forth the amino acid sequence of an illustrative CD8α hinge comprising cysteine to serine mutations at positions 27 and 44 (C27S; C44S)

SEQ ID NO: 6 sets forth the amino acid sequence of an illustrative CD8α transmembrane (TM) region.

SEQ ID NOs: 7-20 set forth amino acid sequences of additional illustrative hinge regions.

SEQ ID NOs: 21-31 set forth the amino acid sequence of various linkers.

SEQ ID NOs: 32-56 set forth the amino acid sequence of protease cleavage sites and self-cleaving polypeptide cleavage sites.

In the foregoing sequences, X, if present, refers to any amino acid or the absence of an amino acid.

### DETAILED DESCRIPTION

### A. OVERVIEW

The present disclosure provides modified CCR polypeptides, fusion polypeptides, and genetically modified cells, wherein the CCR comprises a modified hinge region. In particular embodiments, cells modified to express CARs and CCRs contemplated herein, display reduced antigen-independent signaling in the absence of a CAR target antigen, compared to a polypeptide, fusion polypeptide, or cell comprising a non-modified CCR hinge region. More particularly, cells modified to express CARs and CCRs contemplated herein, display reduced signaling when in the presence of the CCR antigen and absence of the CAR antigen.

In other words, the genetically modified cells described herein are activated against target cells expressing both the CAR and CCR antigens, while displaying little to no activation against cells expressing the CCR antigen alone. Without wishing to be bound by any particular theory, it is contemplated that the decreased signaling in the presence of the CCR antigen and absence of the CAR antigen is achieved by reducing disulfide bonding between the CCR and CAR hinge regions.

In particular embodiments, CCRs are useful for their ability to augment CAR signaling. Moreover, CCRs can be designed to target a different antigen than a CAR, thus potentially increasing target cell specificity and/or reducing the occurrence of refractory target cells. Importantly, the dual-antigen approach, in its purest form, may provide the ability to utilize boolean logic (AND; OR; NOT) to target particular subsets of cells and not others. The usefulness of this approach in cell therapies is highlighted by the difficulty in finding cancer-specific antigens (scc e.g., Newick K. et al., Annu. Rev. Med. 2017;68:139-152). Indeed, many antigens display at least some expression on other cell types and/or elsewhere in the body. Accordingly, one approach to overcome this problem is to differentiate the CAR and CCR antigen specificity.

In particular embodiments, therapies are designed to target cells that only express both the CAR and the CCR antigens. Moreover, by appropriately tuning the level of signaling/activation, targeting of cells that only express one of the antigens is excluded. For example, cells that express 1) the CAR antigen, and 2) both the CAR and CCR antigens, but not cells that only express the CCR antigen are targeted. In other embodiments, therapies are designed to target cells that express 1) the CCR antigen, and 2) both the CCR and CAR antigens, but not cells that only express the CAR antigen. Thus, careful selection and tuning of CAR/CCR combinations and/or fusion polypeptides, and the use of boolean logic, enables targeting of specific cells types (e.g., cancer cells) while minimizing off-target effects, e.g., the targeting of cells that express only the CAR or CCR antigen.

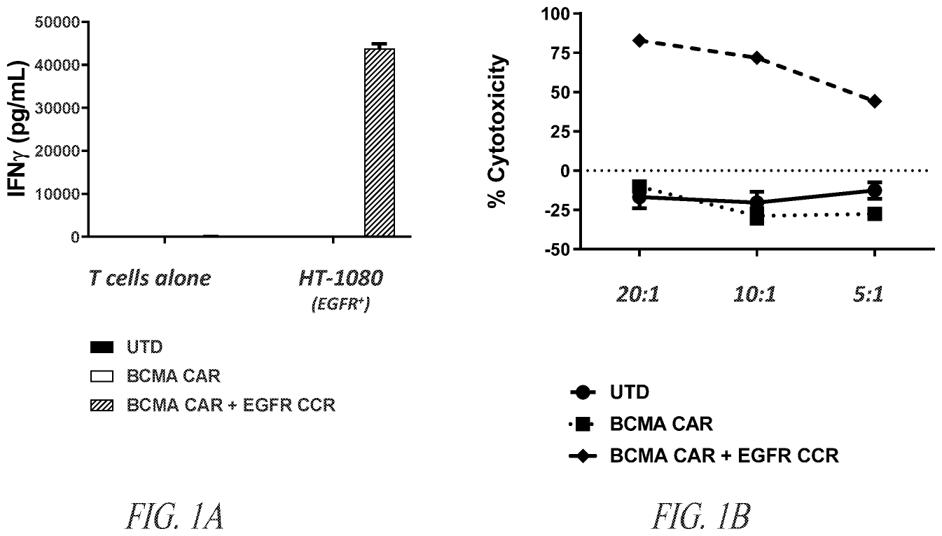

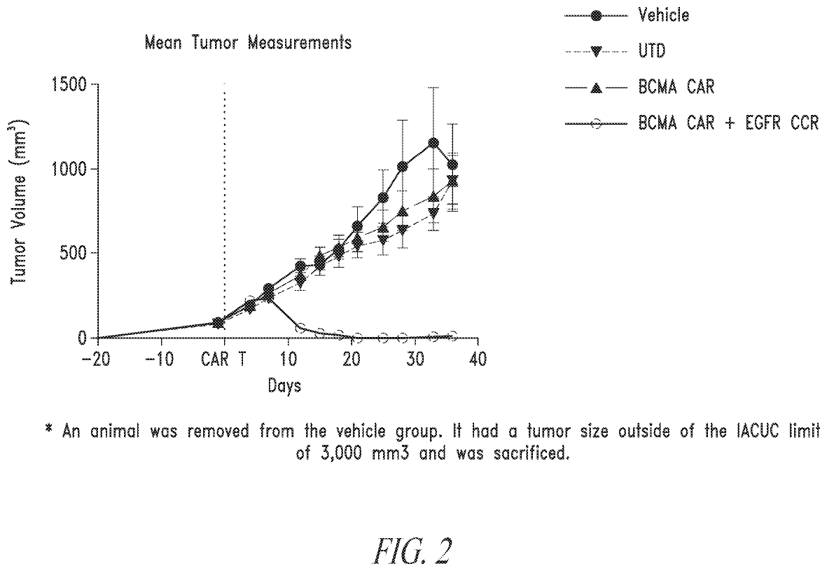

Interestingly, the inventors surprisingly discovered that some CAR/CCR combinations (e.g., fusion polypeptides) exhibit signaling in response to target cells expressing only the CCR antigen, i.e., against target cells that do not express the CAR antigen (Sec, e.g., FIGS. 1A, 1B, and 2). As discussed above, this is surprising because the CCR does not contain a signaling domain, and thus theoretically should not be able to signal on its own. Moreover, if the CCR antigen is one that is widely expressed on other cell types (e.g., an EGFR antigen), activation/signaling in the presence of the CCR antigen alone significantly reduces the benefits of a dual-antigen approach. Additionally, such signaling may also induce T cell dysfunction and/or killing of off-target cell types.

Without wishing to be bound by any particular theory, the inventors herein contemplate that certain CCRs are able to interact with the corresponding CAR via disulfide bonding of cysteine residues between the CAR and CCR hinge regions which induces CAR-mediated T cell signaling in the absence CAR antigen. This CCR antigen-dependent, CAR antigen-independent, signaling results in T cell dysfunction and reduces T-cell efficacy. As described and exemplified herein, the inventors surprisingly discovered that the problem of CAR antigen-independent signaling can in part be driven by the CCR and such signaling can be solved by mutating particular cysteine residues within the CCR hinge region. In particular embodiments, immune effector cells expressing a CAR and a CCR comprising a modified hinge region contemplated herein is activated against cells that express both CAR and CCR antigens, while eliminating/reducing activation against cells expressing only the CCR antigen.

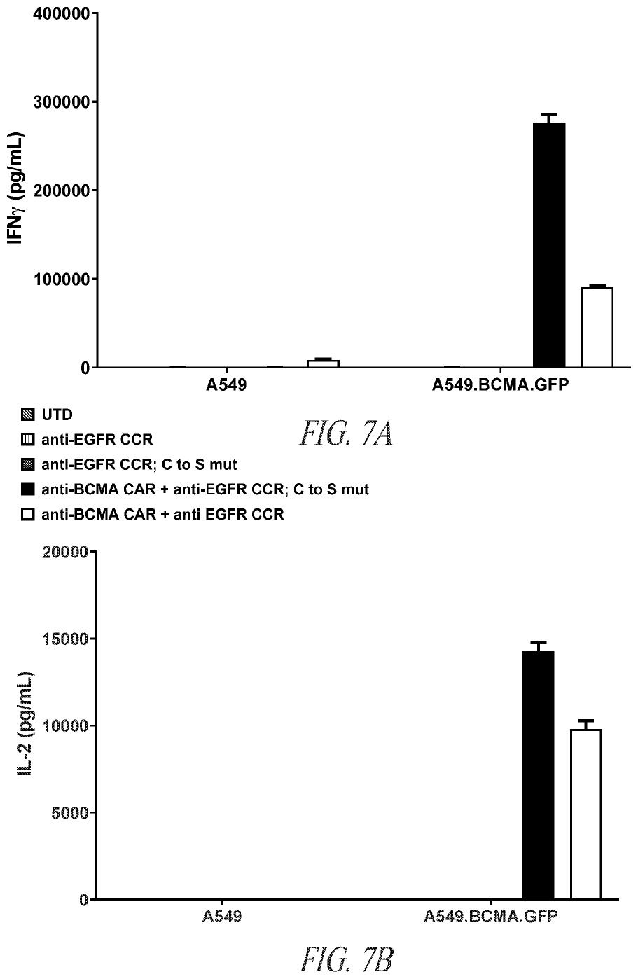

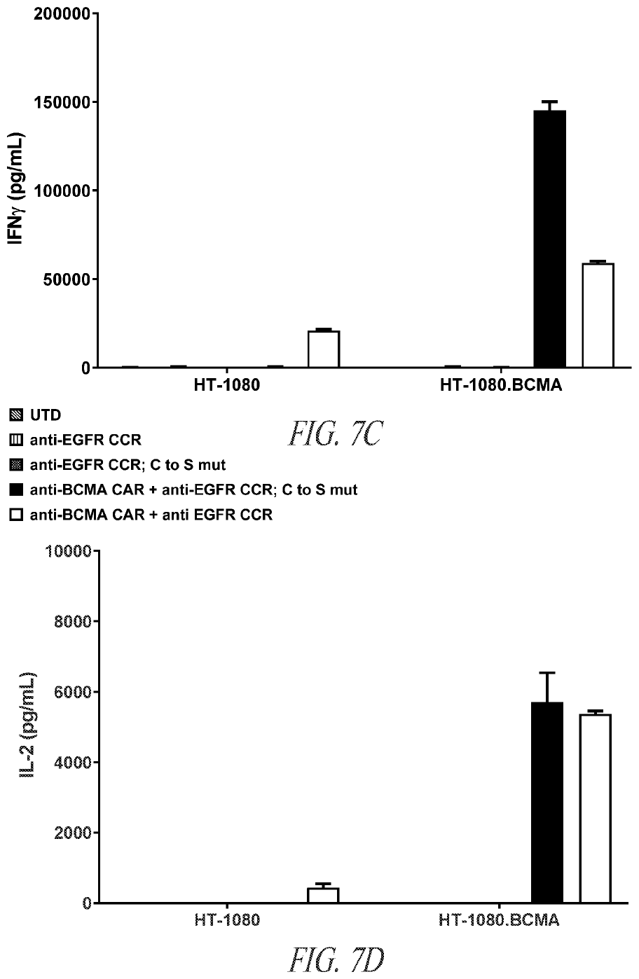

Thus, the present invention provides, in part, CAR and CCR polypeptides, fusion polypeptides, and genetically modified cells that express a CAR and a CCR, wherein the CCR comprises a modified hinge region. In particular, the polypeptides, fusion polypeptides, or genetically modified cells described herein, display reduced T cell signaling in the absence of the CAR antigen compared to a fusion polypeptide or cell comprising nonmodified CCR hinge region. More particularly, the improved polypeptides, fusion polypeptides, or genetically modified cells described herein surprisingly reduce CAR antigen-independent signaling, while maintaining or increasing T cell signaling in the presence of both antigens (sce e.g., FIGS. 7A-7D).

In various embodiments, a fusion polypeptide is provided comprising a chimeric antigen receptor (CAR); a polypeptide cleavage signal; and a chimeric co-stimulatory receptor (CCR) comprising a hinge region modified to reduce CCR co-stimulation of T cell signaling in the absence of CAR antigen, as compared to a CCR comprising a non-modified hinge region.

In various embodiments, a fusion polypeptide is provided comprising a chimeric antigen receptor (CAR); a polypeptide cleavage signal; and a chimeric co-stimulatory receptor (CCR) comprising a hinge region comprising one or more cysteines substituted with another amino acid. In some embodiments, the one or more cysteine substitutions within the CCR hinge region reduces CCR antigen-mediated stimulation of T cell signaling in the absence of CAR antigen, compared to a CCR comprising a non-modified hinge region. In some embodiments, the one or more cysteine substitutions within the CCR hinge region reduces CAR/CCR association in the absence of CAR antigen, compared to a CCR comprising a non-modified hinge region.

In various embodiments, the CCR and/or CAR comprise modified hinge region(s). In some embodiments, the modified CAR and/or CCR hinge regions comprise one or more cysteines substituted with a different amino acid (e.g., serine(s) or alanine(s)). In some embodiments, the one or more cysteines substituted within the CAR and/or CCR hinge region(s) reduce(s) CCR antigen-mediated stimulation of T cell signaling in the absence of CAR antigen compared to a CAR and/or CCR comprising a non-modified hinge region. In some embodiments, the one or more cysteines substitutions within the CAR and/or CCR hinge region(s) reduce(s) CAR/CCR association in the absence of CAR antigen compared to a CAR and/or CCR comprising a non-modified hinge region.

In various embodiments, the CAR and CCR hinge regions are derived from the same or different proteins. In some embodiments, the hinge region is derived from the CD8α hinge region (e.g., SEQ ID NO: 2). In some embodiments, the CD8α hinge region of the CCR and/or CAR comprises an amino acid substitution at position 27 of SEQ ID NO: 2 (e.g., SEQ ID NO: 3). In some embodiments, the CD8α hinge region of the CCR and/or CAR comprises an amino acid substitution at position 44 of SEQ ID NO: 2 (e.g., SEQ ID NO: 4). In some embodiments, the CD8α hinge region of the CCR and/or CAR comprises amino acid substitutions at positions 27 and 44 of SEQ ID NO: 2 (e.g., SEQ ID NO: 5).

In various embodiments, a fusion polypeptide is provided comprising a chimeric antigen receptor (CAR), a polypeptide cleavage signal, and a chimeric co-stimulatory receptor (CCR), wherein the CAR comprises a first antibody or antigen-specific binding fragment thereof, a first hinge region, a first transmembrane domain, and a first intracellular co-stimulatory domain, and a primary signaling domain; and the CCR comprises a second antibody or antigen-specific binding fragment thereof, a second hinge region, a second transmembrane domain, and a second intracellular co-stimulatory domain; wherein the second hinge domain of the CCR comprises one or more cysteine residues substituted for a different amino acid.

In various embodiments, a polynucleotide encoding a fusion polypeptide as described herein is provided. In some embodiments, vector comprising a polynucleotide as described herein is provided. In some embodiments, a cell that expresses a fusion polypeptide as contemplated herein is provided. In some embodiments, the cell is a genetically engineered host cell, a hematopoietic cell, a hematopoietic stem cell, a hematopoietic progenitor cell, CD34⁺ cell, an immune effector cell, a T cell, a CD3⁺ cell, a CD4⁺ cell, a CD8⁺ cell, a cytotoxic T lymphocyte (CTL), a tumor infiltrating lymphocyte (TIL), a helper T cell, a αβ-T cell, γδ-T cell, a natural killer (NK) cell, a natural killer T (NKT) cell, or a macrophage.

In various embodiments, a composition comprising a cell as described herein is provided. In some embodiments, the composition comprises a pharmaceutically acceptable carrier.

In various embodiments, a method of treating a cancer in a subject in need thereof is provided, comprising administering to the subject a therapeutically effective amount of a composition as described herein.

In various embodiments, a method of generating a population of cells that express a fusion polypeptide as described herein is provided, comprising introducing into the population of cells a polynucleotide or vector as described herein.

In various embodiments, a method of reducing CCR antigen-mediated stimulation of T cell signaling in the absence of CAR antigen, compared to a CCR comprising a non-modified hinge region is provided, the method comprising: obtaining CAR and CCR polypeptides each having hinge domain, optionally wherein the CAR and CCR are expressed as a fusion polypeptide; substituting one or more cysteine residues within the CCR hinge domain for another residue, thereby producing a modified CCR; and expressing the CAR and modified CCR in a cell. In various embodiments, a method of reducing CCR co-stimulation of T cell signaling in cells expressing both a CAR and a CCR is provided, the method comprising: a) obtaining CAR and CCR polypeptides cach having hinge domain, optionally wherein the CAR and CCR are expressed as a fusion polypeptide; b) substituting one or more cysteine residues within the CCR hinge domain for another residue, thereby producing a modified CCR; and c) expressing the CAR and modified CCR in a cell.

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d cd., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeck, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lanc, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. DEFINITIONS

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by up to 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or clements. By “consisting of” is meant including, and limited to, whatever follows the phrase “consisting of.” Thus, the phrase “consisting of” indicates that the listed elements are required or mandatory, and that no other elements may be present. By “consisting essentially of” is meant to include any elements listed after the phrase and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

Additional definitions are set forth throughout this disclosure.

### C. CHIMERIC ANTIGEN RECEPTORS (CARS) AND MODIFIED CHIMERIC CO-STIMULATORY RECEPTORS (CCRS)

In various embodiments, immune effector cells are modified to express a CAR and a CCR in order to redirect cytotoxicity of immune effector cells toward cancer cells expressing particular antigens and synergistically increase the effectiveness of the immune effector cell therapy. CARs are molecules that combine antibody-based specificity for a desired antigen with T cell receptor primary and co-stimulatory signaling components. Unlike CARs, CCRs are molecules that combine antibody-based specificity for a desired antigen with a T cell receptor co-stimulatory domain but that lack a primary signaling domain. As used herein, the term, “chimeric,” describes being composed of parts of different proteins or DNAs from different origins.

In particular embodiments, an immune effector cell expresses a CAR comprising an antigen binding domain and a CCR comprising a different antigen binding domain. CARs comprise an extracellular antigen binding domain, a hinge region, a transmembrane domain, an intracellular co-stimulatory domain, and a primary signaling domain and CCRs comprise an extracellular antigen binding domain, a hinge region, a transmembrane domain, and an intracellular co-stimulatory domain, and lack a primary signaling domain. Engagement of the antigen binding domain of the CCR with the antigen on the surface of a target cell results in clustering of the CCR and delivers a co-stimulatory signal to the CAR-containing cell. The main characteristic of CCRs is their ability to further redirect or fine tune immune effector cell specificity in an MHC independent manner and enhance the immune effector cell response in the presence of a CAR, preferably in the presence of a CAR antigen.

In various embodiments, a CCR comprises an extracellular binding domain that comprises an antigen-specific binding domain; a modified hinge region; a transmembrane domain; and a co-stimulatory signaling domain, but not a primary signaling domain.

In preferred embodiments, the CCR hinge region comprises one or more cysteine residues substituted with one or more other amino acid residues (e.g., one or more serine residues).

Illustrative examples of CAR and CCR componentry are further disclosed, infra.

### 1. Binding Domain

In particular embodiments, the CARs and CCRs comprise an extracellular binding domain that comprises an antibody or antigen binding fragment thereof that specifically binds to a particular antigen expressed on a target cell, e.g., a cancer cell. As used herein, the terms, “binding domain,” “extracellular domain,” “extracellular binding domain,” “antigen-specific binding domain,” and “extracellular antigen specific binding domain,” are used interchangeably and provide a CAR and CCR with the ability to specifically bind to the target antigen of interest. The binding domain may be derived either from a natural, synthetic, semi-synthetic, or recombinant source.

The terms “specific binding affinity” or “specifically binds” or “specifically bound” or “specific binding” or “specifically targets” as used herein, describe binding of an antibody or antigen binding fragment thereof (or a CAR and CCR comprising the same) to an antigen at greater binding affinity than background binding. A binding domain (or a CAR and CCR comprising a binding domain or a fusion protein containing a binding domain) “specifically binds” to an antigen if it binds to or associates with the antigen with an affinity or Ka (i.e., an equilibrium association constant of a particular binding interaction with units of 1/M) of, for example, greater than or equal to about 10⁵ M⁻¹. In certain embodiments, a binding domain (or a fusion protein thereof) binds to a target with a Ka greater than or equal to about 10⁶ M⁻¹, 10⁷ M⁻¹, 10⁸ M⁻¹, 10⁹ M⁻¹, 10¹⁰ M⁻¹, 10¹¹ M⁻¹, 10¹² M⁻¹, or 10¹³ M⁻¹. “High affinity” binding domains (or single chain fusion proteins thereof) refers to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, at least 10¹³ M⁻¹, or greater.

Alternatively, affinity may be defined as an equilibrium dissociation constant (Kd) of a particular binding interaction with units of M (e.g., 10⁻⁵ M to 10⁻¹³ M, or less). Affinities of binding domain polypeptides and CAR and CCR proteins according to the present disclosure can be readily determined using conventional techniques, e.g., by competitive ELISA (enzyme-linked immunosorbent assay), or by binding association, or displacement assays using labeled ligands, or using a surface-plasmon resonance device such as the Biacore T100, which is available from Biacore, Inc., Piscataway, NJ, or optical biosensor technology such as the EPIC system or EnSpire that are available from Corning and Perkin Elmer respectively (see also, e.g., Scatchard et al. (1949) Ann. N.Y. Acad. Sci. 51:660; and U.S. Pat. Nos. 5,283,173; 5,468,614, or the equivalent).

In one embodiment, the affinity of specific binding is about 2 times greater than background binding, about 5 times greater than background binding, about 10 times greater than background binding, about 20 times greater than background binding, about 50 times greater than background binding, about 100 times greater than background binding, or about 1000 times greater than background binding or more.

In particular embodiments, the extracellular binding domain of a CAR and CCR comprises an antibody or antigen binding fragment thereof. An “antibody” refers to a binding agent that is a polypeptide comprising at least a light chain or heavy chain immunoglobulin variable region which specifically recognizes and binds an epitope of an antigen, such as a peptide, lipid, polysaccharide, or nucleic acid containing an antigenic determinant, such as those recognized by an immune cell. An “isolated antibody or antigen binding fragment thereof” is one which has been identified and separated and/or recovered from a component of its natural environment.

An “antigen (Ag)” refers to a compound, composition, or substance that can stimulate the production of antibodies or a T cell response in an animal, including compositions (such as one that includes a cancer-specific protein) that are injected or absorbed into an animal. An antigen reacts with the products of specific humoral or cellular immunity, including those induced by heterologous antigens, such as the disclosed antigens.

An “epitope” or “antigenic determinant” refers to the region of an antigen to which a binding agent binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation.

Antibodies include chimeric antibodies (for example, humanized murine antibodies), heteroconjugate antibodies (such as, bispecific antibodies) and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

As would be understood by the skilled person and as described elsewhere herein, a complete antibody comprises two heavy chains and two light chains. Each heavy chain consists of a variable region and a first, second, and third constant region, while cach light chain consists of a variable region and a constant region. Mammalian heavy chains are classified as α, δ, ε, γ, and μ. Mammalian light chains are classified as λ or κ. Immunoglobulins comprising the α, δ, ε, γ, and μ heavy chains are classified as immunoglobulin (Ig)A, IgD, IgE, IgG, and IgM. The complete antibody forms a “Y” shape. The stem of the Y consists of the second and third constant regions (and for IgE and IgM, the fourth constant region) of two heavy chains bound together and disulfide bonds (inter-chain) are formed in the hinge. Heavy chains γ, α and δ have a constant region composed of three tandem (in a line) Ig domains, and a hinge region for added flexibility; heavy chains μ and ε have a constant region composed of four immunoglobulin domains. The second and third constant regions are referred to as “CH2 domain” and “CH3 domain”, respectively. Each arm of the Y includes the variable region and first constant region of a single heavy chain bound to the variable and constant regions of a single light chain. The variable regions of the light and heavy chains are responsible for antigen binding.

Light and heavy chain variable regions contain a “framework” region interrupted by three hypervariable regions, also called “complementarity-determining regions” or “CDRs.” The CDRs can be defined or identified by conventional methods, such as by sequence according to Kabat et al. (Wu, T T and Kabat, E. A., J Exp Med. 132(2):211-50, (1970); Borden, P. and Kabat E. A., PNAS, 84: 2440-2443 (1987); (see, Kabat et al., Sequences of Proteins of Immunological Interest, U.S. Department of Health and Human Services, 1991, which is hereby incorporated by reference), or by structure according to Chothia et al (Chothia, C. and Lesk, A. M., J Mol. Biol., 196(4): 901-917 (1987), Chothia, C. et al, Nature, 342: 877 - 883 (1989)).

Illustrative examples of rules for predicting light chain CDRs include: CDR-L1 starts at about residue 24, is preceded by a Cys, is about 10-17 residues, and is followed by a Trp (typically Trp-Tyr-Gln, but also, Trp-Leu-Gln, Trp-Phe-Gln, Trp-Tyr-Leu); CDR-L2 starts about 16 residues after the end of CDR-L1, is generally preceded by Ile-Tyr, but also, Val-Tyr, Ile-Lys, Ile-Phe, and is 7 residues; and CDR-L3 starts about 33 residues after the end of CDR-L2, is preceded by a Cys, is 7-11 residues, and is followed by Phe-Gly-XXX-Gly (XXX is any amino acid) (SEQ ID NO: 58).

Illustrative examples of rules for predicting heavy chain CDRs include: CDR-H1 starts at about residue 26, is preceded by Cys-XXX-XXX-XXX (SEQ ID NO: 59), is 10-12 residues and is followed by a Trp (typically Trp-Val, but also, Trp-Ile, Trp-Ala); CDR-H2 starts about 15 residues after the end of CDR-H1, is generally preceded by Leu-Glu-Trp-Ile-Gly (SEQ ID NO: 60), or a number of variations, is 16-19 residues, and is followed by Lys/Arg-Leu/Ile/Val/Phe/Thr/Ala-Thr/Ser/Ile/Ala; and CDR-H3 starts about 33 residues after the end of CDR-H2, is preceded by Cys-XXX-XXX (typically Cys-Ala-Arg), is 3 to 25 residues, and is followed by Trp-Gly-XXX-Gly (SEQ ID NO: 61).

In one embodiment, light chain CDRs and the heavy chain CDRs are determined according to the Kabat method

In one embodiment, light chain CDRs and the heavy chain CDR2 and CDR3 are determined according to the Kabat method, and heavy chain CDR1 is determined according to the AbM method, which is a comprise between the Kabat and Clothia methods, see e.g., Whitelegg N & Rees A R, Protein Eng. 2000 Dec; 13(12):819-24 and Methods Mol Biol. 2004;248:51-91. Programs for predicting CDRs are publicly available, e.g., AbYsis (www.bioinf.org.uk/abysis/).

The sequences of the framework regions of different light or heavy chains are relatively conserved within a species, such as humans. The framework region of an antibody, that is the combined framework regions of the constituent light and heavy chains, serves to position and align the CDRs in three-dimensional space. The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, the CDRs located in the variable domain of the heavy chain of the antibody are referred to as CDRH1, CDRH2, and CDRH3, whereas the CDRs located in the variable domain of the light chain of the antibody are referred to as CDRL1, CDRL2, and CDRL3. Antibodies with different specificities (i.e., different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “VL” or “VL” refer to the variable region of an immunoglobulin light chain, including that of an antibody, Fv, scFv, dsFv, Fab, or other antibody fragment as contemplated herein. References to “VH” or “VH” refer to the variable region of an immunoglobulin heavy chain, including that of an antibody, Fv, scFv, dsFv, Fab, or other antibody fragment as contemplated herein.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “chimeric antibody” has framework residues from one species, such as human, and CDRs (which generally confer antigen binding) from another species, such as a mouse. In particular preferred embodiments, a CAR and/or CCR comprises antigen-specific binding domain that is a chimeric antibody or antigen binding fragment thereof.

In particular embodiments, the antibody is a human antibody (such as a human monoclonal antibody) or fragment thereof that specifically binds to a human polypeptide. Human antibodies can be constructed by combining Fv clone variable domain sequence(s) selected from human-derived phage display libraries with known human constant domain sequences(s) as described above. Alternatively, human monoclonal antibodies may be made by the hybridoma method. Human myeloma and mouse-human heteromyeloma cell lines for the production of human monoclonal antibodies have been described, for example, by Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991). In addition, transgenic animals (e.g., mice) can be used to produce a full repertoire of human antibodies in the absence of endogenous immunoglobulin production. See, e.g., Jakobovits et al., PNAS USA, 90: 2551 (1993); Jakobovits et al., Nature, 362: 255 (1993); Bruggermann et al., Year in Immunol., 7: 33 (1993). Gene shuffling can also be used to derive human antibodies from non-human, e.g., rodent antibodies, where the human antibody has similar affinities and specificities to the starting non-human antibody. (Sec PCT WO 93/06213 published Apr. 1, 1993). Unlike traditional humanization of non-human antibodies by CDR grafting, this technique provides completely human antibodies, which have no FR or CDR residues of non-human origin.

In one embodiment, a CAR and/or CCR comprises a “humanized” antibody. A humanized antibody is an immunoglobulin including a human framework region and one or more CDRs from a non-human (for example a mouse, rat, or synthetic) immunoglobulin. The non-human immunoglobulin providing the CDRs is termed a “donor.” and the human immunoglobulin providing the framework is termed an “acceptor.” In one embodiment, all the CDRs are from the donor immunoglobulin in a humanized immunoglobulin. Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. Humanized or other monoclonal antibodies can have additional conservative amino acid substitutions, which have substantially no effect on antigen binding or other immunoglobulin functions. Humanized antibodies can be constructed by means of genetic engineering (scc for example, U.S. Pat. No. 5,585,089).

Antibodies include antigen binding fragments thereof, such as a Camel Ig, a Llama Ig, an Alpaca Ig. Ig NAR, a Fab′ fragment, a F(ab′)2 fragment, a bispecific Fab dimer (Fab2), a trispecific Fab trimer (Fab3), an Fv, an single chain Fv protein (“scFv”), a bis-scFv, (scFv)2, a minibody, a diabody, a triabody, a tetrabody, a disulfide stabilized Fv protein (“dsFv”), and a single-domain antibody (sdAb, a camelid VHH, Nanobody) and portions of full length antibodies responsible for antigen binding. Antibodies also include: polyclonal and monoclonal antibodies and antigen binding fragments thereof; murine antibodies, camelid antibodies, and human antibodies, and antigen binding fragments thereof; and chimeric antibodies, heteroconjugate antibodies, and humanized antibodies, and antigen binding fragments thereof. See also, Pierce Catalog and Handbook, 1994-1995 (Pierce Chemical Co., Rockford, IL); Kuby, J., Immunology, 3rd Ed., W. H. Freeman & Co., New York, 1997.

A “heavy chain antibody” refers to an antibody that contains two VH domains and no light chains (Riechmann L. et al, J. Immunol. Methods 231:25-38 (1999); WO94/04678; WO94/25591; U.S. Pat. No. 6,005,079). A “camelid antibody” refers to an antibody isolated from a Camel, Alpaca, or Llama that contains two VH domains and no light chains. A “humanized VHH” or “humanized camelid antibody” refers to a non-human VHH or camelid antibody that has undergone humanization to reduce potential immunogenicity of the antibody in human recipients.

“IgNAR” of “immunoglobulin new antigen receptor” refers to class of antibodies from the shark immune repertoire that consist of homodimers of one variable new antigen receptor (VNAR) domain and five constant new antigen receptor (CNAR) domains. IgNARs represent some of the smallest known immunoglobulin-based protein scaffolds and are highly stable and possess efficient binding characteristics. The inherent stability can be attributed to both (i) the underlying Ig scaffold, which presents a considerable number of charged and hydrophilic surface exposed residues compared to the conventional antibody VH and VL domains found in murine antibodies; and (ii) stabilizing structural features in the complementary determining region (CDR) loops including inter-loop disulphide bridges, and patterns of intra-loop hydrogen bonds.

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment which contains a complete antigen-binding site. In one embodiment, a two-chain Fv species consists of a dimer of one heavy- and one light-chain variable domain in tight, non-covalent association. In a single-chain Fv (scFv) species, one heavy-and one light-chain variable domain can be covalently linked by a flexible peptide linker such that the light and heavy chains can associate in a “dimeric” structure analogous to that in a two-chain Fv species. It is in this configuration that the three hypervariable regions (HVRs) of each variable domain interact to define an antigen-binding site on the surface of the VH-VL dimer. Collectively, the six HVRs confer antigen-binding specificity to the antibody. However, even a single variable domain (or half of an Fv comprising only three HVRs specific for an antigen) has the ability to recognize and bind antigen, although at a lower affinity than the entire binding site.

The Fab fragment contains the heavy- and light-chain variable domains and also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known. Bispecific Fab dimers (Fab2) have two Fab′ fragments, cach binding a different antigen. Trispecific Fab trimers (Fab3) have three Fab′ fragments, cach binding a different antigen.

The term “diabodies” refers to antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies may be bivalent or bispecific. Diabodies are described more fully in, for example, EP 404,097; WO 1993/01161; Hudson et al., Nat. Med. 9:129-134 (2003); and Hollinger et al., PNAS USA 90: 6444-6448 (1993). Triabodies and tetrabodies are also described in Hudson et al., Nat. Med. 9:129-134 (2003).

“Single domain antibody” or “sdAb” or “nanobody” refers to an antibody fragment that consists of the variable region of an antibody heavy chain (VH domain) or the variable region of an antibody light chain (VL domain) (Holt, L., et al, Trends in Biotechnology, 21(11): 484-490).

“Single-chain Fv” or “scFv” antibody fragments comprise the VH and VL domains of antibody, wherein these domains are present in a single polypeptide chain and in either orientation (e.g., VL-VH or VH-VL). Generally, the scFv polypeptide further comprises a polypeptide linker between the VH and VL domains which enables the scFv to form the desired structure for antigen binding. For a review of scFv, sec, e.g., Pluckthün, in The Pharmacology of Monoclonal Antibodies, vol. 113, Rosenburg and Moore eds., (Springer-Verlag, New York, 1994), pp. 269-315.

In preferred embodiments, the antigen-specific binding fragment is an scFv or VHH. In particular embodiments, the scFv is a murine, human or humanized scFv. Single chain antibodies may be cloned form the V region genes of a hybridoma specific for a desired target. The production of such hybridomas has become routine. A technique which can be used for cloning the variable region heavy chain (VH) and variable region light chain (VL) has been described, for example, in Orlandi et al., PNAS, 1989; 86: 3833-3837.

In various embodiments, the antigen-specific binding domain of the CAR (e.g., a first binding domain) and/or CCR (e.g., a second binding domain) binds a target antigen selected from the group consisting of: alpha folate receptor (FRα), αvβ6 integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA 10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

In particular embodiments, the antigen-specific binding domain is an scFv or VHH.

In particular embodiments, the antigen-specific binding domain is an scFv that binds a human BCMA or EGFR polypeptide.

### 2. Linkers

In certain embodiments, CARs and/or CCRs comprise linker residues between the various domains, e.g., added for appropriate spacing and conformation of the molecule. In particular embodiments the linker is a variable region linking sequence. A “variable region linking sequence,” is an amino acid sequence that connects the VH and VI, domains and provides a spacer function compatible with interaction of the two sub-binding domains so that the resulting polypeptide retains a specific binding affinity to the same target molecule as an antibody that comprises the same light and heavy chain variable regions. In particular embodiments, CARs and/or CCRs comprise one, two, three, four, or five or more linkers. In particular embodiments, the length of a linker is about 1 to about 25 amino acids, about 5 to about 20 amino acids, or about 10 to about 20 amino acids, or any intervening length of amino acids. In some embodiments, the linker is 1, 2, 3, 4, 5, 6, 7, 8, 9, 10, 11, 12, 13, 14, 15, 16, 17, 18, 19, 20, 21, 22, 23, 24, 25, or more amino acids long.

Illustrative examples of linkers include glycine polymers (G)ₙ; glycine-serine polymers (G₁₋₅S₁₋₅)ₙ, where n is an integer of at least one, two, three, four, or five; glycine-alanine polymers; alanine-serine polymers; and other flexible linkers known in the art. Glycine and glycine-serine polymers are relatively unstructured, and therefore may be able to serve as a neutral tether between domains of fusion proteins such as the CARs or CCRs described herein. Glycine accesses significantly more phi-psi space than even alanine, and is much less restricted than residues with longer side chains (see Scheraga, Rev. Computational Chem. 11173-142 (1992)). The ordinarily skilled artisan will recognize that design of a CAR or CCR in particular embodiments can include linkers that are all or partially flexible, such that the linker can include a flexible linker as well as one or more portions that confer less flexible structure to provide for a desired CAR or CCR structure.

Other exemplary linkers include, but are not limited to the following amino acid sequences: DGGGS (SEQ ID NO: 21); TGEKP (SEQ ID NO: 22) (see, e.g., Liu et al., PNAS 5525-5530 (1997)); GGRR (SEQ ID NO: 23) (Pomerantz et al. 1995, supra); (GGGGS)ₙ wherein =1, 2, 3, 4 or 5 (SEQ ID NO: 24) (Kim et al., PNAS 93, 1156-1160 (1996.); EGKSSGSGSESKVD (SEQ ID NO: 25) (Chaudhary et al., 1990, Proc. Natl. Acad. Sci. U.S.A. 87:1066-1070); KESGSVSSEQLAQFRSLD (SEQ ID NO: 26) (Bird et al., 1988, Science 242:423-426), GGRRGGGS (SEQ ID NO: 27); LRQRDGERP (SEQ ID NO: 28); LRQKDGGGSERP (SEQ ID NO: 29); LRQKD(GGGS)2 ERP (SEQ ID NO: 30). Alternatively, flexible linkers can be rationally designed using a computer program capable of modeling both DNA-binding sites and the peptides themselves (Desjarlais & Berg, PNAS 90:2256-2260 (1993), PNAS 91:11099-11103 (1994) or by phage display methods. In one embodiment, the linker comprises the following amino acid sequence: GSTSGSGKPGSGEGSTKG (SEQ ID NO: 31) (Cooper et al., Blood, 101(4): 1637-1644 (2003)).

### 3. Spacer Domain

In particular embodiments, the binding domain of a CAR and/or CCR is followed by one or more “spacer domains,” which refers to the region that moves the antigen binding domain away from the effector cell surface to enable proper cell/cell contact, antigen binding and activation (Patel et al., Gene Therapy, 1999; 6: 412-419). The spacer domain may be derived either from a natural, synthetic, semi-synthetic, or recombinant source. In certain embodiments, a spacer domain is a portion of an immunoglobulin, including, but not limited to, one or more heavy chain constant regions, e.g., CH2 and CH3. The spacer domain can include the amino acid sequence of a naturally occurring immunoglobulin hinge region or an altered immunoglobulin hinge region.

In one embodiment, the spacer domain comprises the CH2 and CH3 domains of IgG1, IgG4, or IgD.

### 4. Hinge Domain

The terms “hinge”, “hinge domain”, and “hinge region” are used interchangeably herein and refer to a flexible domain that plays a role in positioning the antigen binding domain away from the effector cell surface to enable proper cell/cell contact, antigen binding and activation. The binding domain of a CAR and/or CCR is generally followed by one or more hinge domains between the binding domain and the transmembrane domain (TM). The hinge domain may be derived either from a natural, synthetic, semi-synthetic, or recombinant source. The hinge domain can include the amino acid sequence of a naturally occurring immunoglobulin hinge region or an altered immunoglobulin hinge region. In some embodiments, the hinge domain comprises a spacer domain.

As used herein, the terms “altered hinge region”, “modified hinge region”, and “modified hinge domain” are used interchangeably and refer to (a) a naturally occurring hinge region with up to 30% amino acid changes (e.g., up to 25%, 20%, 15%, 10%, or 5% amino acid substitutions or deletions), (b) a portion of a naturally occurring hinge region that is at least 10 amino acids (e.g., at least 12, 13, 14 or 15 amino acids) in length with up to 30% amino acid changes (e.g., up to 25%, 20%, 15%, 10%, or 5% amino acid substitutions or deletions), or (c) a portion of a naturally occurring hinge region that comprises the core hinge region (which may be 4, 5, 6, 7, 8, 9, 10, 11, 12, 13, 14, or 15, or at least 4, 5, 6, 7, 8, 9, 10, 11, 12, 13, 14, or 15 amino acids in length).

In preferred embodiments, the modified hinge region comprises an amino acid sequence having at least 85%, 86%, 87%, 88%, 89%, 90%, 91%, 92%, 93%, 94%, 95%, 96%, 97%, 98%, or 99% amino acid identity to a suitable hinge domain/region as described herein and/or known in the art. In some embodiments, the modified hinge region comprising a hinge sequence as described herein having 4 or fewer, 3 or fewer, or 2 or fewer amino acid substitutions and/or deletions.

In certain embodiments, one or more cysteine residues in a naturally occurring hinge region may be substituted by one or more other amino acid residues (e.g., one or more serine residues). In particular embodiments, an altered hinge region comprises substitution of a proline residue by another amino acid residue (e.g., a serine residue).

Illustrative hinge domains suitable for use in the CARs and/or CCRs contemplated in particular embodiments herein include the hinge region derived from the extracellular regions of type 1 membrane proteins including, but not limited to, CD8α, CD4, CD28 and CD7, which may be wild-type hinge regions from these molecules or may be altered. In one embodiment, the hinge is a PD-1 hinge or CD152 hinge. In another embodiment, the hinge domain comprises a naturally occurring immunoglobin hinge region, e.g., an IgG1, IgG2, IgG3, or IgG4 hinge. In another embodiment the hinge domain comprises an IgG1 hinge/CH2/CH3, an IgG1 hinge/CH3/hinge/M1, an IgG4 hinge/CH2/CH3, or an IgG4 hinge/CH2.

In some embodiments, the CD8α hinge region comprises the amino acid sequence set forth in SEQ ID NO: 2, or functional fragment or alteration thereof.

In some embodiments, the CD4 hinge region comprises the amino acid sequence set forth in SEQ ID NO: 7, or functional fragment or alteration thereof.

In some embodiments, the CD28 hinge region comprises the amino acid sequence set forth in SEQ ID NO: 8, or functional fragment or alteration thereof.

In some embodiments, the CD7 hinge region comprises the amino acid sequence set forth in SEQ ID NO: 9, or functional fragment or alteration thereof.

In some embodiments, the CD152 hinge region comprises the amino acid sequence set forth in SEQ ID NO: 10, or functional fragment or alteration thereof.

In some embodiments, the PD-1 hinge region comprises the amino acid sequence set forth in SEQ ID NO: 11, or functional fragment or alteration thereof.

In some embodiments, the IgG1 hinge region comprises the amino acid sequence set forth in SEQ ID NO: 12, or functional fragment or alteration thereof.

In some embodiments, the IgG2 hinge region comprises the amino acid sequence set forth in SEQ ID NO: 13, or functional fragment or alteration thereof.

In some embodiments, the IgG3 hinge region comprises the amino acid sequence set forth in SEQ ID NO: 14, or functional fragment or alteration thereof.

In some embodiments, the IgG4 hinge region comprises the amino acid sequence set forth in SEQ ID NO: 15, or functional fragment or alteration thereof.

In some embodiments, the IgG1 hinge/CH2/CH3 hinge region comprises the amino acid sequence set forth in SEQ ID NO: 16, or functional fragment or alteration thereof.

In some embodiments, the IgG1 Hinge-CH3-Hinge-M1 hinge region comprises the amino acid sequence set forth in SEQ ID NO: 17, or functional fragment or alteration thereof.

In some embodiments, the IgG4 hinge/CH2/CH3 hinge region comprises the amino acid sequence set forth in SEQ ID NO: 18, or functional fragment or alteration thereof.

In some embodiments, the IgG4 hinge/CH2 hinge region comprises the amino acid sequence set forth in SEQ ID NO: 19, or functional fragment or alteration thereof.

In some embodiments, the PD-1 hinge region comprises the amino acid sequence set forth in SEQ ID NO: 20, or functional fragment or alteration thereof.

In various embodiments, the fusion polypeptides described herein comprise a modified first and/or second hinge region comprising an amino acid sequence having at least 85%, 86%, 87%, 88%, 89%, 90%, 91%, 92%, 93%, 94%, 95%, 96%, 97%, 98%, or 99% amino acid identity to any one of the hinge domain/regions as described above. In some embodiments, the modified first and/or second hinge region comprises a hinge sequence, as described above, having 4 or fewer, 3 or fewer, or 2 or fewer amino acid substitutions and/or deletions.

In certain embodiments, one or more cysteine residues in a hinge region/domain may be substituted by one or more other amino acid residues to produce a modified hinge domain. In some embodiments, the modified hinge domain comprises one or more cysteine residues substituted with serine(s) or alanine(s). In another embodiment, the modified hinge domain comprises one or more cysteine residues substituted with serine(s). In another embodiment, the modified CD8a hinge domain comprises one or more cysteine residues substituted with alanine(s). In some embodiments, the CCR hinge domain is modified. In some embodiments, the CAR hinge domain is not modified. In some embodiments, both the CAR and CCR hinge domains are modified.

In a preferred embodiment, the hinge domain comprises a CD8α hinge region/domain. In one embodiment, the hinge domain comprises the amino acid residues as set forth in SEQ ID NO: 2. In another embodiment, the CAR and/or CCR comprises a modified CD8α hinge domain. In another embodiment, the CCR comprises a modified CD8α hinge domain, and the CAR optionally comprises a wild-type hinge sequence. In another embodiment, the modified CD8α hinge domain comprises one or more cysteine residues substituted with one or more other amino acid residues. In another embodiment, the modified CD8α hinge domain comprises one or more cysteine residues substituted with serine(s) or alanine(s). In another embodiment, the modified CD8α hinge domain comprises one or more cysteine residues substituted with serine(s). In another embodiment, the modified CD8α hinge domain comprises one or more cysteine residues substituted with alanine(s).

In one embodiment, the modified CD8α hinge domain comprises cysteine to serine substitution at position 27 of SEQ ID NO: 2. In another embodiment, the modified CD8α hinge domain comprises the amino acid residues as set forth in SEQ ID NO: 3. In a particular embodiment, the modified CD8α hinge domain comprises an amino acid sequence having at least 85%, 86%, 87%, 88%, 89%, 90%, 91%, 92%, 93%, 94%, 95%, 96%, 97%, 98%, or 99% amino acid identity to the sequence set forth in SEQ ID NO: 3. In particular embodiments, the modified CD8α hinge domain comprises an amino acid sequence having at least 90%, 93%, 95%, or 97% identity to the sequence set forth in SEQ ID NO: 3. In some embodiments, the modified CD8α hinge domain comprises 4 or fewer, 3 or fewer, or 2 or fewer amino acid substitutions and/or deletions.

In one embodiment, the modified CD8α hinge domain comprises cysteine to serine substitution at position 44 of SEQ ID NO: 2. In another embodiment, the modified CD8α hinge domain comprises the amino acid residues as set forth in SEQ ID NO: 4. In a particular embodiment, the modified CD8α hinge domain comprises an amino acid sequence having at least 85%, 86%, 87%, 88%, 89%, 90%, 91%, 92%, 93%, 94%, 95%, 96%, 97%, 98%, or 99% amino acid identity to the sequence set forth in SEQ ID NO: 4. In particular embodiments, the modified CD8α hinge domain comprises an amino acid sequence having at least 90%, 93%, 95%, or 97% identity to the sequence set forth in SEQ ID NO: 4. In some embodiments, the modified CD8α hinge domain comprises 4 or fewer, 3 or fewer, or 2 or fewer amino acid substitutions and/or deletions.

In one embodiment, the modified CD8α hinge domain comprises cysteine to serine substitution at positions 27 and 44 of SEQ ID NO: 2. In another embodiment, the modified CD8α hinge domain comprises the amino acid residues as set forth in SEQ ID NO: 5. In a particular embodiment, the modified CD8α hinge domain comprises an amino acid sequence having at least 85%, 86%, 87%, 88%, 89%, 90%, 91%, 92%, 93%, 94%, 95%, 96%, 97%, 98%, or 99% amino acid identity to the sequence set forth in SEQ ID NO: 5. In particular embodiments, the modified CD8α hinge domain comprises an amino acid sequence having at least 90%, 93%, 95%, or 97% identity to the sequence set forth in SEQ ID NO: 5. In some embodiments, the modified CD8α hinge domain comprises 4 or fewer, 3 or fewer, or 2 or fewer amino acid substitutions and/or deletions.

### 5. Transmembrane (TM) Domain

The “transmembrane domain” is the portion of a CAR and/or CCR that fuses the extracellular binding portion and intracellular signaling domain and anchors the CAR and/or CCR to the plasma membrane of the immune effector cell. The TM domain may be derived either from a natural, synthetic, semi-synthetic, or recombinant source. The TM domain may be derived from (i.e., comprises at least the transmembrane region(s) of) the alpha or beta chain of the T-cell receptor, CD3δ, CD3ε, CD3γ, CD3ζ, CD4, CD5, CD8α, CD9, CD16, CD22, CD27, CD28, CD33, CD37, CD45, CD64, CD80, CD86, CD134, CD137, CD152, CD154, and PD1. In a particular embodiment, the TM domain is synthetic and predominantly comprises hydrophobic residues such as leucine and valine.

In one embodiment, the CARs and/or CCRs comprise a TM domain derived from, PD1, CD152, CD28, or CD8α. In another embodiment, a CAR and/or CCR comprises a TM domain derived from, PD1, CD152, CD28, or CD8α and a short oligo- or polypeptide linker, preferably between 1, 2, 3, 4, 5, 6, 7, 8, 9, or 10 amino acids in length that links the TM domain and the intracellular signaling or co stimulatory domains of the CAR or CCR as the case may be. A glycine-serine based linker provides a particularly suitable linker.

For example, in one non-limiting and merely illustrative embodiment the transmembrane domain comprises a CD8α transmembrane domain as set forth in SEQ ID NO: 6. In a particular embodiment, the CD8α transmembrane domain comprises an amino acid sequence having at least 85%, 86%, 87%, 88%, 89%, 90%, 91%, 92%, 93%, 94%, 95%, 96%, 97%, 98%, or 99% amino acid identity to the sequence set forth in SEQ ID NO: 6.

### 6. Intracellular Signaling Domain

In particular embodiments, the CARs comprise one or more intracellular signaling domains. An “intracellular signaling domain.” refers to the part of a CAR that participates in transducing the message of effective CAR binding to a human antigen into the interior of the immune effector cell to elicit effector cell function, e.g., activation, cytokine production, proliferation and cytotoxic activity, including the release of cytotoxic factors to the CAR-bound target cell, or other cellular responses elicited with antigen binding to the extracellular CAR domain.

The term “effector function” refers to a specialized function of an immune effector cell. Effector function of the T cell, for example, may be cytolytic activity or help or activity including the secretion of a cytokine. Thus, the term “intracellular signaling domain” refers to the portion of a protein which transduces the effector function signal and that directs the cell to perform a specialized function. While usually the entire intracellular signaling domain can be employed, in many cases it is not necessary to use the entire domain. To the extent that a truncated portion of an intracellular signaling domain is used, such truncated portion may be used in place of the entire domain as long as it transduces the effector function signal. The term intracellular signaling domain is meant to include any truncated portion of the intracellular signaling domain sufficient to transducing effector function signal.

It is known that signals generated through the TCR alone are insufficient for full activation of the T cell and that a secondary or co-stimulatory signal is also required. Thus, T cell activation can be said to be mediated by two distinct classes of intracellular signaling domains: primary signaling domains that initiate antigen-dependent primary activation through the TCR (e.g., a TCR/CD3 complex) and co-stimulatory signaling domains that act in an antigen-independent manner to provide a secondary or co-stimulatory signal. In preferred embodiments, a CAR comprises an intracellular signaling domain that comprises one or more “co-stimulatory signaling domain” and a “primary signaling domain.”

Primary signaling domains regulate primary activation of the TCR complex either in a stimulatory way, or in an inhibitory way. Primary signaling domains that act in a stimulatory manner may contain signaling motifs which are known as immunoreceptor tyrosine-based activation motifs or ITAMs.

Illustrative examples of ITAM containing primary signaling domains that are useful in particular embodiments include those derived from FcRγ, FcRβ, CD3γ, CD3δ, CD3ε, CD3ζ, CD22, CD79a, CD79b, and CD66d. In particular preferred embodiments, a CAR comprises a CD3ζ primary signaling domain and one or more co-stimulatory signaling domains. The intracellular primary signaling and co-stimulatory signaling domains may be linked in any order in tandem to the carboxyl terminus of the transmembrane domain.

In particular embodiments, CARs and/or CCRs comprise one or more co-stimulatory signaling domains to enhance the efficacy and expansion of T cells expressing CAR and CCR receptors. As used herein, the term, “co-stimulatory signaling domain,” or “co-stimulatory domain”, refers to an intracellular signaling domain of a co-stimulatory molecule. Co-stimulatory molecules are cell surface molecules other than antigen receptors or Fc receptors that provide a second signal required for efficient activation and function of T lymphocytes upon binding to antigen. Illustrative examples of such co-stimulatory molecules include TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, TNFR2, and ZAP70. In one embodiment, a CAR and/or CCR comprises one or more co-stimulatory signaling domains selected from the group consisting of CD28, CD137, and CD134, and a CD35 primary signaling domain.

In another embodiment, a CAR and/or CCR comprises CD28 and CD137 co-stimulatory signaling domains and a CD3ζ primary signaling domain.

In yet another embodiment, a CAR and/or CCR comprises CD28 and CD134 co-stimulatory signaling domains and a CD3ζ primary signaling domain.

In one embodiment, a CAR and/or CCR comprises CD137 and CD134 co-stimulatory signaling domains and a CD3ζ primary signaling domain.

In one embodiment, a CAR and/or CCR comprises a CD137 co-stimulatory signaling domain and a CD3ζ primary signaling domain.

In one embodiment, a CAR and/or CCR comprises a CD134 co-stimulatory signaling domain and a CD3ζ primary signaling domain.

In one embodiment, a CAR and/or CCR comprises a CD28 co-stimulatory signaling domain and a CD3ζ primary signaling domain.

### D. Illustrative Embodiments of CARs and CCRs

In one embodiment, a CAR comprises an antibody or antigen-specific binding fragment thereof that binds an antigen; a hinge region; a transmembrane domain; one or more intracellular co-stimulatory signaling domains from a co-stimulatory molecule; and a primary signaling domain.

In one embodiment, a CAR comprises an antibody or antigen-specific binding fragment thereof that binds an antigen expressed on a cancer cell; a hinge region; a transmembrane domain derived from a polypeptide selected from the group consisting of: alpha or beta chain of the T-cell receptor, CDδ, CD3ε, CDγ, CD3ζ, CD4, CD5, CD8α, CD9, CD16, CD22, CD27, CD28, CD33, CD37, CD45, CD64, CD80, CD86, CD134, CD137, CD152, CD154, AMN1, and PD1; one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from FcRγ, FcRβ, CD3γ, CD3δ, CD3ε, CD3ζ, CD22, CD79a, CD79b, and CD66d.

In one embodiment, the CAR comprises a hinge domain selected from the group consisting of: a CD8α hinge, a CD4 hinge, a CD28 hinge, a CD7 hinge, a CD152 hinge, a PD1 hinge, an IgG1 hinge, an IgG2 hinge, an IgG3 hinge, an IgG4 hinge, an IgG1 hinge/CH2/CH3, an IgG1 hinge/CH3/hinge/M1, an IgG4 hinge/CH2/CH3, and an IgG4 hinge/CH2.

In one embodiment, a CAR comprises an antibody or antigen-specific binding fragment thereof that binds an antigen expressed on a cancer; a hinge domain selected from the group consisting of: a CD8α hinge, a CD4 hinge, a CD28 hinge, a CD7 hinge, a CD152 hinge, a PD1 hinge, an IgG1 hinge, an IgG2 hinge, an IgG3 hinge, an IgG4 hinge, an IgG1 hinge/CH2/CH3, an IgG1 hinge/CH3/hinge/M1, an IgG4 hinge/CH2/CH3, and an IgG4 hinge/CH2; a transmembrane domain derived from a polypeptide selected from the group consisting of: alpha or beta chain of the T-cell receptor, CDδ, CD3ε, CDγ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70; and a primary signaling domain from FcRγ, FcRβ, CD3γ, CD3δ, CD3ε, CD3ζ, CD22, CD79a, CD79b, and CD66d.

In a particular embodiment, a CAR comprises an antibody or antigen-specific binding fragment thereof that binds an antigen expressed on a cancer; a hinge domain comprising a CD8α polypeptide; a CD8α transmembrane domain comprising a polypeptide linker (e.g., a Tail-CD8 linker) of about 3 to about 10 amino acids; a CD137 (4-1BB) intracellular co-stimulatory signaling domain; and a CD3ζ primary signaling domain.

In a particular embodiment, a CAR comprises an antibody or antigen-specific binding fragment thereof that binds an antigen expressed on a cancer; a hinge domain comprising a CD8α polypeptide; a CD8α transmembrane domain comprising a polypeptide linker of about 3 to about 10 amino acids (e.g., a Tail-CD8 linker); a CD134 intracellular co-stimulatory signaling domain; and a CD3ζ primary signaling domain.

In a particular embodiment, a CAR comprises an antibody or antigen-specific binding fragment thereof that binds an antigen expressed on a cancer; a hinge domain comprising a CD8α polypeptide; a CD8α transmembrane domain comprising a polypeptide linker of about 3 to about 10 amino acids (e.g., a Tail-CD8 linker); a CD28 intracellular co-stimulatory signaling domain; and a CD3ζ primary signaling domain.

In various embodiments, the CAR antibody or antigen-specific binding fragment thereof is an scFv or a VHH. In some embodiments, the CAR antibody or antigen-specific binding fragment thereof is an scFv. In some embodiments, the CAR antibody or antigen-specific binding fragment thereof is an VHH.

In any one of the embodiments described herein, the CAR hinge region may be modified to reduced antigen-independent T cell signaling as described herein. In any one of the embodiments described herein, the hinge region may be modified to reduce association with a CCR in the absence of CAR antigen, compared to a CAR comprising a non-modified hinge region.

In one embodiment, the modified CAR hinge region comprises one or more cysteines substituted with a different amino acid. In another embodiment, the one or more cysteine residues are substituted with serine(s) or alanine(s). In a preferred embodiment, the modified hinge region is a CD8α hinge region. In another embodiment, the modified CD8α hinge region is derived from SEQ ID NO: 2. In another embodiment, the modified CD8α hinge region comprises an amino acid substitution at position 27 of SEQ ID NO: 2 (i.e., see SEQ ID NO: 3). In another embodiment, the modified CD8α hinge region comprises an amino acid substitution at position 44 of SEQ ID NO: 2 (i.e., see SEQ ID NO: 4). In another embodiment, the modified CD8α hinge region comprises an amino acid substitution at positions 27 and 44 of SEQ ID NO: 2 (i.e., see SEQ ID NO: 5).

In any of the above embodiments, the one or more cysteine substitutions within the CAR hinge region reduces CCR co-stimulation of T cell signaling in the absence of CAR antigen, compared to a CAR comprising a non-modified hinge region. In any one of the above embodiments, the one or more cysteine substitutions within the CAR hinge region reduces association with the CCR in the absence of CAR antigen, compared to a CAR comprising a non-modified hinge region.

In one embodiment, a CCR comprises an antibody or antigen-specific binding fragment thereof that binds an antigen; a hinge region; a transmembrane domain; and one or more intracellular co-stimulatory signaling domains from a co-stimulatory molecule.

In one embodiment, a CCR comprises an antibody or antigen-specific binding fragment thereof that binds an antigen expressed on a cancer; a modified hinge region comprising one or more cysteines substituted with a different amino acid; a transmembrane domain derived from a polypeptide selected from the group consisting of: alpha or beta chain of the T-cell receptor, CD3δ, CD3ε, CD3γ, CD3ζ, CD4, CD5, CD8α, CD9, CD16, CD22, CD27, CD28, CD33, CD37, CD45, CD64, CD80, CD86, CD134, CD137, CD152, CD154, AMN1, and PD1; and one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, and ZAP70.

In one embodiment, the CCR comprises a modified hinge region selected from the group consisting of: a CD8α hinge, a CD4 hinge, a CD28 hinge, a CD7 hinge, a CD152 hinge, a PD1 hinge, an IgG1 hinge, an IgG2 hinge, an IgG3 hinge, an IgG4 hinge, an IgG1 hinge/CH2/CH3, an IgG1 hinge/CH3/hinge/M1, an IgG4 hinge/CH2/CH3 and an IgG4 hinge/CH2.

In one embodiment, a CCR comprises an antibody or antigen-specific binding fragment thereof that binds an antigen expressed on a cancer; a modified hinge region selected from the group consisting of: a CD8α hinge, a CD4 hinge, a CD28 hinge, a CD7 hinge, a CD152 hinge, a PD1 hinge, an IgG1 hinge, an IgG2 hinge, an IgG3 hinge, an IgG4 hinge, an IgG1 hinge/CH2/CH3, an IgG1 hinge/CH3/hinge/M1, an IgG4 hinge/CH2/CH3, and an IgG4 hinge/CH2; a transmembrane domain derived from a polypeptide selected from the group consisting of: alpha or beta chain of the T-cell receptor, CD3δ, CD3ε, CD3γ, CD3ζ, CD4, CD5, CD8α, CD9, CD 16, CD22, CD27, CD28, CD33, CD37, CD45, CD64, CD80, CD86, CD 134, CD137, CD152, CD154, AMN1, and PD1; a short oligo- or polypeptide linker, preferably between 1, 2, 3, 4, 5, 6, 7, 8, 9, or 10 amino acids in length that links the TM domain to the intracellular signaling domain of the CAR; and one or more intracellular co-stimulatory signaling domains from a co-stimulatory molecule selected from the group consisting of: TLR1, TLR2, TLR3, TLR4, TLR5, TLR6, TLR7, TLR8, TLR9, TLR10, CARD11, CD2, CD7, CD27, CD28, CD30, CD40, CD54 (ICAM), CD83, CD134 (OX40), CD137 (4-1BB), CD278 (ICOS), DAP10, LAT, NKD2C, SLP76, TRIM, TNFR2, and ZAP70.

In a particular embodiment, a CCR comprises an scFv or VHH that binds an antigen expressed on a cancer; a modified hinge region comprising a CD8α polypeptide as described herein; a CD8α transmembrane domain comprising a polypeptide linker (e.g., a Tail-CD8 linker) of about 3 to about 10 amino acids; and a CD137 intracellular co-stimulatory signaling domain.

In a particular embodiment, a CCR comprises an scFv or VHH that binds an antigen expressed on a cancer; a modified hinge region comprising a CD8α polypeptide as described herein; a CD8α transmembrane domain comprising a polypeptide linker of about 3 to about 10 amino acids (e.g., a Tail-CD8 linker); and a CD134 intracellular co-stimulatory signaling domain.

In a particular embodiment, a CCR comprises an scFv or VHH that binds an antigen expressed on a cancer; a modified hinge region comprising a CD8α polypeptide as described herein; a CD8α transmembrane domain comprising a polypeptide linker of about 3 to about 10 amino acids (e.g., a Tail-CD8 linker); and a CD28 intracellular co-stimulatory signaling domain.

In various embodiments, the CCR antibody or antigen-specific binding fragment thereof is an scFv or a VHH. In some embodiments, the CCR antibody or antigen-specific binding fragment thereof is an scFv. In some embodiments, the CCR antibody or antigen-specific binding fragment thereof is an VHH.

In any one of the embodiments described herein, the CCR hinge region may be modified to reduced antigen-independent T cell signaling as described herein. In any one of the embodiments described herein, the CCR hinge region may be modified to reduce association with a CAR in the absence of CAR antigen, compared to a CCR comprising a non-modified hinge region.

In one embodiment, the modified CCR hinge region comprises one or more cysteines substituted with a different amino acid. In another embodiment, the one or more cysteine residues are substituted with serine(s) or alanine(s). In a preferred embodiment, the modified hinge region is a CD8α hinge region. In another embodiment, the modified CD8α hinge region is derived from SEQ ID NO: 2. In another embodiment, the modified CD8α hinge region comprises an amino acid substitution at position 27 of SEQ ID NO: 2 (i.e., see SEQ ID NO: 3). In another embodiment, the modified CD8α hinge region comprises an amino acid substitution at position 44 of SEQ ID NO: 2 (i.e., see SEQ ID NO: 4). In another embodiment, the modified CD8α hinge region comprises an amino acid substitution at positions 27 and 44 of SEQ ID NO: 2 (i.e., see SEQ ID NO: 5).

In any of the above embodiments, the one or more cysteine substitutions within the CCR hinge region reduces CCR co-stimulation of T cell signaling in the absence of CAR antigen, compared to a CCR comprising a non-modified hinge region. In any one of the above embodiments, the one or more cysteine substitutions within the CCR hinge region reduces association with the CAR in the absence of CAR antigen, compared to a CCR comprising a non-modified hinge region.

In various embodiments, the antigen-specific binding domain of the CAR and/or CCR binds a target antigen selected from the group consisting of: alpha folate receptor (FRα), αᵥβ₆ integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1). In particular embodiments, the antigen-specific binding domain is an scFv or VHH. In particular embodiments, the antigen-specific binding domain is an scFv. In particular embodiments, the antigen-specific binding domain is an VHH. In particular embodiments, the antigen-specific binding domain is an scFv that binds a human BCMA or EGFR polypeptide.

In various embodiments, the antigen-specific binding domain of the CAR binds a target antigen selected from the group consisting of: alpha folate receptor (FRα), αᵥβ₆ integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA 10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1).

In various embodiments, the antigen-specific binding domain of the CAR binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands.

In various embodiments, the antigen-specific binding domain of the CAR binds a human BCMA polypeptide.

In various embodiments, the antigen-specific binding domain of the CCR binds a target antigen selected from the group consisting of: alpha folate receptor (FRα), αᵥβ₆ integrin, B cell maturation antigen (BCMA), B7-H3 (CD276), B7-H6, carbonic anhydrase IX (CAIX), CCR1, CD16, CD19, CD20, CD22, CD30, CD33, CD37, CD38, CD44, CD44v6, CD44v7/8, CD70, CD79a, CD79b, CD123, CD133, CD135 (also known as fmc like tyrosine kinase 3; FLT3), CD138, CD171, carcinoembryonic antigen (CEA), Claudin-6 (CLDN6), C-type lectin-like molecule-1 (CLL-1), CD2 subset 1 (CS-1), chondroitin sulfate proteoglycan 4 (CSPG4), cutaneous T cell lymphoma-associated antigen 1 (CTAGE1), epidermal growth factor receptor (EGFR), epidermal growth factor receptor variant III (EGFRvIII), epithelial glycoprotein 2 (EGP2), epithelial glycoprotein 40 (EGP40), epithelial cell adhesion molecule (EPCAM), ephrin type-A receptor 2 (EPHA2), fibroblast activation protein (FAP), Fc Receptor Like 5 (FCRL5), fetal acetylcholinesterase receptor (AchR), ganglioside G2 (GD2), ganglioside G3 (GD3), Glypican-3 (GPC3), EGFR family including ErbB2 (HER2), IL-11Rα, IL-13Rα2, Kappa, cancer/testis antigen 2 (LAGE-1A), Lambda, Lewis-Y (LeY), L1 cell adhesion molecule (L1-CAM), Leukocyte immunoglobulin-like receptor subfamily B member 2 (LILRB2); melanoma antigen gene (MAGE)-A1, MAGE-A3, MAGE-A4, MAGE-A6, MAGEA10, melanoma antigen recognized by T cells 1 (MelanA or MART1), Mesothelin (MSLN), MUC1, MUC16, neural cell adhesion molecule (NCAM), cancer/testis antigen 1 (NY-ESO-1), polysialic acid; placenta-specific 1 (PLAC1), preferentially expressed antigen in melanoma (PRAME), prostate stem cell antigen (PSCA), prostate-specific membrane antigen (PSMA), receptor tyrosine kinase-like orphan receptor 1 (ROR1), synovial sarcoma, X breakpoint 2 (SSX2), Survivin, tumor associated glycoprotein 72 (TAG72), T-cell immunoglobulin and mucin-domain containing-3 (TIM-3), tumor endothelial marker 1 (TEM1/CD248), tumor endothelial marker 7-related (TEM7R), trophoblast glycoprotein (TPBG), NKG2D ligands, vascular endothelial growth factor receptor 2 (VEGFR2), and Wilms tumor 1 (WT-1). In various embodiments, the antigen-specific binding domain of the CCR binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands

In various embodiments, the antigen-specific binding domain of the CCR binds a human EGFR polypeptide, an EGFRvIII polypeptide, a TAG72 polypeptide, or a CD20 polypeptide.

In various embodiments, the antigen-specific binding domain of the CCR binds a human EGFR polypeptide. In various embodiments, the antigen-specific binding domain of the CCR binds a human EGFRvIII polypeptide. In various embodiments, the antigen-specific binding domain of the CCR binds a human TAG72 polypeptide. In various embodiments, the antigen-specific binding domain of the CCR binds a human CD20 polypeptide.

In particular embodiments, the antigen-specific binding domain of the CCR is an scFv or a VHH.

In various embodiments, the CAR comprises a first antibody or antigen-specific binding fragment thereof; and the CCR comprises a second antibody or antigen-specific binding fragment thereof. In some embodiments, the first and second antibody or antigen-specific binding fragments bind the same target antigen. In some embodiments, the first and second antibody or antigen-specific binding fragments bind different epitopes on the same target antigen. In some embodiments, the first and second antibody or antigen-specific binding fragments bind different target antigens.

In various embodiments, the antigen-specific binding domain of the CAR binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the antigen-specific binding domain of the CCR binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands.

In various embodiments, the antigen-specific binding domain of the CAR binds a target antigen selected from the group consisting of: B cell maturation antigen (BCMA), CD20, CD33, CD70, CD79a, CD79b, CD123, CD133, C-type lectin-like molecule-1 (CLL-1), epidermal growth factor receptor (EGFR), epidermal growth factor receptor variant III (EGFRvIII), EGFR family including ErbB2 (HER2), MAGE-A4, MUC1, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the antigen-specific binding domain of the CCR binds a target antigen selected from the group consisting of: B cell maturation antigen (BCMA), CD20, epidermal growth factor receptor (EGFR), epidermal growth factor receptor variant III (EGFRvIII), and TAG72.

In various embodiments, the antigen-specific binding domain of the CAR binds a target antigen selected from the group consisting of: B cell maturation antigen (BCMA), CD33, CD79a, CD79b, C-type lectin-like molecule-1 (CLL-1), MAGE-A4, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the antigen-specific binding domain of the CCR binds a target antigen selected from the group consisting of: B cell maturation antigen (BCMA), CD20, epidermal growth factor receptor (EGFR), epidermal growth factor receptor variant III (EGFRvIII), and TAG72.

In various embodiments, the antigen-specific binding domain of the CAR binds a target antigen is B cell maturation antigen (BCMA), CD33, CD79a, CD79b, C-type lectin-like molecule-1 (CLL-1), MAGE-A4, MUC16, cancer/testis antigen 1 (NY-ESO-1), preferentially expressed antigen in melanoma (PRAME), receptor tyrosine kinase-like orphan receptor 1 (ROR1), tumor associated glycoprotein 72 (TAG72), and NKG2D ligands; and the antigen-specific binding domain of the CCR binds a target antigen selected from the group consisting of: B cell maturation antigen (BCMA), CD20, epidermal growth factor receptor (EGFR), epidermal growth factor receptor variant III (EGFRvIII), and TAG72.

In various embodiments, the antigen-specific binding domain of the CAR binds a human BCMA polypeptide; and the antigen-specific binding domain of the CCR binds a human EGFR polypeptide.

In various embodiments, the antigen-specific binding domain of the CAR binds a human BCMA polypeptide; and the antigen-specific binding domain of the CCR binds a human EGFRvIII polypeptide.

In various embodiments, the antigen-specific binding domain of the CAR binds a human BCMA polypeptide; and the antigen-specific binding domain of the CCR binds a human CD20 polypeptide.

In various embodiments, the antigen-specific binding domain of the CAR binds a human BCMA polypeptide; and the antigen-specific binding domain of the CCR binds a human TAG72 polypeptide.

In various embodiments, the antigen-specific binding domain of the CAR binds a human CD33 polypeptide; and the antigen-specific binding domain of the CCR binds a human EGFR polypeptide.

In various embodiments, the antigen-specific binding domain of the CAR binds a human CD33 polypeptide; and the antigen-specific binding domain of the CCR binds a human EGFRVIII polypeptide.

In various embodiments, the antigen-specific binding domain of the CAR binds a human CD33 polypeptide; and the antigen-specific binding domain of the CCR binds a human CD20 polypeptide.

In various embodiments, the antigen-specific binding domain of the CAR binds a human CD33 polypeptide; and the antigen-specific binding domain of the CCR binds a human TAG72 polypeptide.

In various embodiments, the antigen-specific binding domain of the CAR binds a human CD79a polypeptide; and the antigen-specific binding domain of the CCR binds a human EGFR polypeptide.

In various embodiments, the antigen-specific binding domain of the CAR binds a human CD79a polypeptide; and the antigen-specific binding domain of the CCR binds a human EGFRvIII polypeptide.

In various embodiments, the antigen-specific binding domain of the CAR binds a human CD79a polypeptide; and the antigen-specific binding domain of the CCR binds a human CD20 polypeptide.

In various embodiments, the antigen-specific binding domain of the CAR binds a human CD79a polypeptide; and the antigen-specific binding domain of the CCR binds a human TAG72 polypeptide.

In various embodiments, the antigen-specific binding domain of the CAR binds a human CD79b polypeptide; and the antigen-specific binding domain of the CCR binds a human EGFR polypeptide.

In various embodiments, the antigen-specific binding domain of the CAR binds a human CD79b polypeptide; and the antigen-specific binding domain of the CCR binds a human EGFRvIII polypeptide.

In various embodiments, the antigen-specific binding domain of the CAR binds a human CD79b polypeptide; and the antigen-specific binding domain of the CCR binds a human CD20 polypeptide.

In various embodiments, the antigen-specific binding domain of the CAR binds a human CD79b polypeptide; and the antigen-specific binding domain of the CCR binds a human TAG72 polypeptide.

In various embodiments, the antigen-specific binding domain of the CAR binds a human CLL-1 polypeptide; and the antigen-specific binding domain of the CCR binds a human EGFR polypeptide.

In various embodiments, the antigen-specific binding domain of the CAR binds a human CLL-1 polypeptide; and the antigen-specific binding domain of the CCR binds a human EGFRvIII polypeptide.

In various embodiments, the antigen-specific binding domain of the CAR binds a human CLL-1 polypeptide; and the antigen-specific binding domain of the CCR binds a human CD20 polypeptide.

In various embodiments, the antigen-specific binding domain of the CAR binds a human CLL-1 polypeptide; and the antigen-specific binding domain of the CCR binds a human TAG72 polypeptide.

In various embodiments, the antigen-specific binding domain of the CAR binds a human MAGE-A4 polypeptide; and the antigen-specific binding domain of the CCR binds a human EGFR polypeptide.

In various embodiments, the antigen-specific binding domain of the CAR binds a human MAGE-A4 polypeptide; and the antigen-specific binding domain of the CCR binds a human EGFRvIII polypeptide.

In various embodiments, the antigen-specific binding domain of the CAR binds a human MAGE-A4 polypeptide; and the antigen-specific binding domain of the CCR binds a human CD20 polypeptide.

In various embodiments, the antigen-specific binding domain of the CAR binds a human MAGE-A4 polypeptide; and the antigen-specific binding domain of the CCR binds a human TAG72 polypeptide.

In various embodiments, the antigen-specific binding domain of the CAR binds a human MUC16 polypeptide; and the antigen-specific binding domain of the CCR binds a human EGFR polypeptide.

In various embodiments, the antigen-specific binding domain of the CAR binds a human MUC16 polypeptide; and the antigen-specific binding domain of the CCR binds a human EGFRvIII polypeptide.

In various embodiments, the antigen-specific binding domain of the CAR binds a human MUC16 polypeptide; and the antigen-specific binding domain of the CCR binds a human CD20 polypeptide.

In various embodiments, the antigen-specific binding domain of the CAR binds a human MUC16 polypeptide; and the antigen-specific binding domain of the CCR binds a human TAG72 polypeptide.

In various embodiments, the antigen-specific binding domain of the CAR binds a human NY-ESO-1 polypeptide; and the antigen-specific binding domain of the CCR binds a human EGFR polypeptide.

In various embodiments, the antigen-specific binding domain of the binds a human NY-ESO-1 polypeptide; and the antigen-specific binding domain of the CCR binds a human EGFRvIII polypeptide.

In various embodiments, the antigen-specific binding domain of the CAR binds a human NY-ESO-1 polypeptide; and the antigen-specific binding domain of the CCR binds a human CD20 polypeptide.

In various embodiments, the antigen-specific binding domain of the CAR binds a human NY-ESO-1 polypeptide; and the antigen-specific binding domain of the CCR binds a human TAG72 polypeptide.

In various embodiments, the antigen-specific binding domain of the CAR binds a human PRAME polypeptide; and the antigen-specific binding domain of the CCR binds a human EGFR polypeptide.

In various embodiments, the antigen-specific binding domain of the CAR binds a human PRAME polypeptide; and the antigen-specific binding domain of the CCR binds a human EGFRvIII polypeptide.

In various embodiments, the antigen-specific binding domain of the CAR binds a human PRAME polypeptide; and the antigen-specific binding domain of the CCR binds a human CD20 polypeptide.

In various embodiments, the antigen-specific binding domain of the CAR binds a human PRAME polypeptide; and the antigen-specific binding domain of the CCR binds a human TAG72 polypeptide.

In various embodiments, the antigen-specific binding domain of the CAR binds a human ROR1 polypeptide; and the antigen-specific binding domain of the CCR binds a human EGFR polypeptide.

In various embodiments, the antigen-specific binding domain of the CAR binds a human ROR1 polypeptide; and the antigen-specific binding domain of the CCR binds a human EGFRvIII polypeptide.

In various embodiments, the antigen-specific binding domain of the CAR binds a human ROR1 polypeptide; and the antigen-specific binding domain of the CCR binds a human CD20 polypeptide.

In various embodiments, the antigen-specific binding domain of the CAR binds a human ROR1 polypeptide; and the antigen-specific binding domain of the CCR binds a human TAG72 polypeptide.

In various embodiments, the antigen-specific binding domain of the CAR binds a human TAG72 polypeptide; and the antigen-specific binding domain of the CCR binds a human EGFR polypeptide.

In various embodiments, the antigen-specific binding domain of the CAR binds a human TAG72 polypeptide; and the antigen-specific binding domain of the CCR binds a human EGFRvIII polypeptide.

In various embodiments, the antigen-specific binding domain of the CAR binds a human TAG72 polypeptide; and the antigen-specific binding domain of the CCR binds a human CD20 polypeptide.

In various embodiments, the antigen-specific binding domain of the CAR binds a human NKG2D ligand polypeptide; and the antigen-specific binding domain of the CCR binds a human EGFR polypeptide.

In various embodiments, the antigen-specific binding domain of the CAR binds a human NKG2D ligand polypeptide; and the antigen-specific binding domain of the CCR binds a human EGFRvIII polypeptide.

In various embodiments, the antigen-specific binding domain of the CAR binds a human NKG2D ligand polypeptide; and the antigen-specific binding domain of the CCR binds a human CD20 polypeptide.

In various embodiments, the antigen-specific binding domain of the CAR binds a human NKG2D ligand polypeptide; and the antigen-specific binding domain of the CCR binds a human TAG72 polypeptide.

The design of the CARs and CCRs contemplated in particular embodiments enable improved expansion, long-term persistence, and cytotoxic properties in T cells expressing the CARs and CCRs compared to non-modified T cells or T cells modified to express other CARs.

### E. Polypeptides

Various polypeptides, fusion polypeptides, and polypeptide variants are contemplated herein, including, but not limited to, CAR polypeptides, CCR polypeptides, fusion polypeptides thereof and fragments thereof. In particular embodiments, exemplary polypeptides contemplated herein include a CCR comprising a modified hinge region and related fusion polypeptides. In particular, the polypeptides described herein, display reduced T cell signaling in the absence of the CAR antigen, compared to a polypeptide or cell comprising non-modified CCR hinge region. More particularly, the improved polypeptides described herein surprisingly reduce CCR mediated CAR antigen-independent signaling, while increasing T cell signaling in the presence of both CAR and CCR antigens.

“Polypeptide,” “peptide” and “protein” are used interchangeably, unless specified to the contrary, and according to conventional meaning, i.e., as a sequence of amino acids. Polypeptides are not limited to a specific length, e.g., they may comprise a full-length polypeptide or a polypeptide fragment, and may include one or more post-translational modifications of the polypeptide, for example, glycosylations, acetylations, phosphorylations and the like, as well as other modifications known in the art, both naturally occurring and non-naturally occurring.

An “isolated polypeptide” and the like, as used herein, refer to in vitro synthesis, isolation, and/or purification of a peptide or polypeptide molecule from a cellular environment, and from association with other components of the cell, i.e., it is not significantly associated with in vivo substances. In particular embodiments, an isolated polypeptide is a synthetic polypeptide, a semi-synthetic polypeptide, or a polypeptide obtained or derived from a recombinant source.

Polypeptides include “polypeptide variants.” Polypeptide variants may differ from a naturally occurring polypeptide in one or more substitutions, deletions, additions and/or insertions. Such variants may be naturally occurring or may be synthetically generated, for example, by modifying one or more of the above polypeptide sequences. For example, in particular embodiments, it may be desirable to improve the binding affinity and/or other biological properties of a CAR and/or CCR by introducing one or more substitutions, deletions, additions and/or insertions into a binding domain, hinge, TM domain, co-stimulatory signaling domain or primary signaling domain, if present. In particular embodiments, polypeptides include polypeptides having at least about 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% amino acid identity to any of the reference sequences contemplated herein, typically where the variant maintains at least one biological activity of the reference sequence. In particular embodiments, the biological activity is binding affinity. In particular embodiments, the biological activity is cytolytic activity.

Polypeptides include “polypeptide fragments.” Polypeptide fragments refer to a polypeptide, which can be monomeric or multimeric that has an amino-terminal deletion, a carboxyl-terminal deletion, and/or an internal deletion or substitution of a naturally occurring or recombinantly-produced polypeptide. Illustrative examples of biologically active polypeptide fragments include antibody fragments. As used herein, the term “biologically active fragment” or “minimal biologically active fragment” refers to a polypeptide fragment that retains at least 100%, at least 90%, at least 80%, at least 70%, at least 60%, at least 50%, at least 40%, at least 30%, at least 20%, at least 10%, or at least 5% of the naturally occurring polypeptide activity. In preferred embodiments, the biological activity is binding affinity to an idiotype. In certain embodiments, a polypeptide fragment can comprise an amino acid chain at least 5 to about 500 amino acids long. It will be appreciated that in certain embodiments, fragments are at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, or 450 amino acids long. Particularly useful polypeptide fragments include functional domains, including antigen-binding domains or fragments of antibodies.

The polypeptide may also be fused in-frame or conjugated to a linker or other sequence for case of synthesis, purification or identification of the polypeptide (e.g., poly-His), or to enhance binding of the polypeptide to a solid support.

As noted above, in particular embodiments, polypeptides may be altered in various ways including amino acid substitutions, deletions, truncations, and insertions. Methods for such manipulations are generally known in the art. For example, amino acid sequence variants of a reference polypeptide can be prepared by mutations in the DNA. Methods for mutagenesis and nucleotide sequence alterations are well known in the art. See, for example, Kunkel (1985, Proc. Natl. Acad. Sci. USA. 82: 488-492), Kunkel et al., (1987, Methods in Enzymol, 154: 367-382), U.S. Pat. No. 4,873,192, Watson, J. D. et al., (Molecular Biology of the Gene, Fourth Edition, Benjamin/Cummings, Menlo Park, Calif., 1987) and the references cited therein. Guidance as to appropriate amino acid substitutions that do not affect biological activity of the protein of interest may be found in the model of Dayhoff et al., (1978) Atlas of Protein Sequence and Structure (Natl. Biomed. Res. Found., Washington, D.C.).

In certain embodiments, a polypeptide variant comprises one or more conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence, e.g., according to Table 1.

TABLE 1
Amino Acid Codons

| | One | Three | | | | | | |
|---|---|---|---|---|---|---|---|---|
| | letter | letter | | | | | | |
| Amino Acids | code | code | Codons | | | | | |
| Alanine | A | Ala | GCA | GCC | GCG | GCU | | |
| Cysteine | C | Cys | UGC | UGU | | | | |
| Aspartic acid | D | Asp | GAC | GAU | | | | |
| Glutamic acid | E | Glu | GAA | GAG | | | | |
| Phenylalanine | F | Phe | UUC | UUU | | | | |
| Glycine | G | Gly | GGA | GGC | GGG | GGU | | |
| Histidine | H | His | CAC | CAU | | | | |
| Isoleucine | I | Iso | AUA | AUC | AUU | | | |
| Lysine | K | Lys | AAA | AAG | | | | |
| Leucine | L | Leu | UUA | UUG | CUA | CUC | CUG | CUU |
| Methionine | M | Met | AUG | | | | | |
| Asparagine | N | Asn | AAC | AAU | | | | |
| Proline | P | Pro | CCA | CCC | CCG | CCU | | |
| Glutamine | Q | Gln | CAA | CAG | | | | |
| Arginine | R | Arg | AGA | AGG | CGA | CGC | CGG | CGU |
| Serine | S | Ser | AGC | AGU | UCA | UCC | UCG | UCU |

| | | | | | | |
|---|---|---|---|---|---|---|
| Threonine | T | Thr | ACA | ACC | ACG | ACU |
| Valine | V | Val | GUA | GUC | GUG | GUU |

| Tryptophan | W | Trp | UGG | |
|---|---|---|---|---|
| Tyrosine | Y | Tyr | UAC | UAU |

Guidance in determining which amino acid residues can be substituted, inserted, or deleted without abolishing biological activity can be found using computer programs well known in the art, such as DNASTAR, DNA Strider, Geneious, Mac Vector, or Vector NTI software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cysteine, serine, threonine, tyrosine) amino acids. Phenylalanine, tryptophan, and tyrosine are sometimes classified jointly as aromatic amino acids. In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p.224).

As outlined above, amino acid substitutions may be based on the relative similarity of the amino acid side-chain substituents, for example, their hydrophobicity, hydrophilicity, charge, size, and the like.

Polypeptide variants further include glycosylated forms, aggregative conjugates with other molecules, and covalent conjugates with unrelated chemical moieties (e.g., pegylated molecules). Covalent variants can be prepared by linking functionalities to groups which are found in the amino acid chain or at the N- or C-terminal residue, as is known in the art. Variants also include allelic variants, species variants, and muteins. Truncations or deletions of regions which do not affect functional activity of the proteins are also variants.

In particular embodiments, expression of a CAR and a CCR in the same cell is desired. Polynucleotide sequences encoding a CAR and CCR can be separated by an IRES sequence as discussed elsewhere herein.

In preferred embodiments, fusion polypeptides are contemplated herein.

In a particular preferred embodiment, a CAR and CCR can be expressed as a fusion polypeptide that comprises one or more self-cleaving polypeptide sequences that separate a CAR and CCR.

Fusion polypeptides and fusion proteins refer to a polypeptide having at least two, three, four, five, six, seven, eight, nine, or ten or more polypeptide segments. Fusion polypeptides are typically linked C-terminus to N-terminus, although they can also be linked C-terminus to C-terminus, N-terminus to N-terminus, or N-terminus to C-terminus. The polypeptides of the fusion protein can be in any order or a specified order. In one embodiment, a fusion protein comprises a CAR, a polypeptide cleavage signal, and a CCR. In another embodiment, a fusion protein comprises a CCR, a polypeptide cleavage signal, and a CAR.

In one embodiment, a fusion protein comprises a CAR, a polypeptide cleavage signal, and a CCR. In another embodiment, a fusion protein comprises a CCR, a polypeptide cleavage signal, and a CAR.

In particular embodiments, a fusion protein comprises a CAR comprising an scFv that binds an antigen expressed on a cancer, a hinge region, a transmembrane domain, a co-stimulatory domain, and a primary signaling domain; a polypeptide cleavage signal; and a CCR comprising an scFv that binds an antigen, a modified hinge region as described herein, a transmembrane domain, and a co-stimulatory domain. Exemplary domains/regions may be selected from those described herein.

In preferred embodiments, a fusion protein comprises a CAR comprising an scFv that binds an antigen expressed on a cancer, a CD8α hinge, a transmembrane domain, a CD137 co-stimulatory domain, and a CD3ζ primary signaling domain; a polypeptide cleavage signal; and a CCR comprising an scFv that binds an antigen, a modified CD8α hinge as described herein, a transmembrane domain, and a CD28 co-stimulatory domain.

Exemplary polypeptide cleavage signals include polypeptide cleavage recognition sites such as protease cleavage sites, nuclease cleavage sites (e.g., rare restriction enzyme recognition sites, self-cleaving ribozyme recognition sites), and self-cleaving viral oligopeptides (see deFelipe and Ryan, 2004. Traffic, 5(8); 616-26).

Suitable protease cleavages sites and self-cleaving peptides are known to the skilled person (see, e.g., in Ryan et al., 1997. J. Gener. Virol. 78, 699-722; Scymczak et al. (2004) Nature Biotech. 5, 589-594). Exemplary protease cleavage sites include, but are not limited to the cleavage sites of potyvirus NIa proteases (e.g., tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, heparin, thrombin, factor Xa and enterokinase. Due to its high cleavage stringency, TEV (tobacco etch virus) protease cleavage sites are preferred in one embodiment, e.g., EXXYXQ(G/S) (SEQ ID NO: 32), for example, ENLYFQG (SEQ ID NO: 33) and ENLYFQS (SEQ ID NO: 34), wherein X represents any amino acid (cleavage by TEV occurs between Q and G or Q and S).

In particular embodiments, the polypeptide cleavage signal is a viral self-cleaving peptide or ribosomal skipping sequence.

Illustrative examples of ribosomal skipping sequences include, but are not limited to: a 2A or 2A-like site, sequence or domain (Donnelly et al., 2001. J. Gen. Virol. 82:1027-1041). In a particular embodiment, the viral 2A peptide is an aphthovirus 2A peptide, a potyvirus 2A peptide, or a cardiovirus 2A peptide.

In one embodiment, the viral 2A peptide is selected from the group consisting of: a foot-and-mouth disease virus (FMDV) 2A peptide, an equine rhinitis A virus (ERAV) 2A peptide, a Thosea asigna virus (TaV) 2A peptide, a porcine teschovirus-1 (PTV-1) 2A peptide, a Theilovirus 2A peptide, and an encephalomyocarditis virus 2A peptide.

Illustrative examples of 2A sites are provided in Table 2.

TABLE 2

| | |
|---|---|
| SEQ ID NO: 35 | GSGATNFSLLKQAGDVEENPGP |
| SEQ ID NO: 36 | ATNFSLLKQAGDVEENPGP |
| SEQ ID NO: 37 | LLKQAGDVEENPGP |
| SEQ ID NO: 38 | GSGEGRGSLLTCGDVEENPGP |
| SEQ ID NO: 39 | EGRGSLLTCGDVEENPGP |
| SEQ ID NO: 40 | LLTCGDVEENPGP |
| SEQ ID NO: 41 | GSGQCTNYALLKLAGDVESNPGP |
| SEQ ID NO: 42 | QCTNYALLKLAGDVESNPGP |
| SEQ ID NO: 43 | LLKLAGDVESNPGP |
| SEQ ID NO: 44 | GSGVKQTLNFDLLKLAGDVESNPGP |
| SEQ ID NO: 45 | VKQTLNFDLLKLAGDVESNPGP |
| SEQ ID NO: 46 | LLKLAGDVESNPGP |
| SEQ ID NO: 47 | LLNFDLLKLAGDVESNPGP |
| SEQ ID NO: 48 | TLNFDLLKLAGDVESNPGP |
| SEQ ID NO: 49 | LLKLAGDVESNPGP |
| SEQ ID NO: 50 | NFDLLKLAGDVESNPGP |
| SEQ ID NO: 51 | QLLNFDLLKLAGDVESNPGP |
| SEQ ID NO: 52 | APVKQTLNFDLLKLAGDVESNPGP |
| SEQ ID NO: 53 | VTELLYRMKRAETYCPRPLLAIHPT |
| | EARHKQKIVAPVKQT |
| SEQ ID NO: 54 | LNFDLLKLAGDVESNPGP |
| SEQ ID NO: 55 | LLAIHPTEARHKQKIVAPVKQTLNF |
| | DLLKLAGDVESNPGP |
| SEQ ID NO: 56 | EARHKQKIVAPVKQTLNFDLLKLAG |
| | DVESNPGP |

In preferred embodiments, a fusion polypeptide comprises a CAR as described herein, a T2A self-cleaving polypeptide, and a CCR as described herein.

### F. Polynucleotides

In preferred embodiments, a polynucleotide encoding one or more CAR polypeptides, CCR polypeptides, or fusion polypeptide comprising a CAR, 2A peptide, and CCR is provided. As used herein, the terms “polynucleotide” or “nucleic acid” refer to deoxyribonucleic acid (DNA), ribonucleic acid (RNA) and DNA/RNA hybrids. Polynucleotides may be single-stranded or double-stranded and either recombinant, synthetic, or isolated. Polynucleotides include, but are not limited to: pre-messenger RNA (pre-mRNA), messenger RNA (mRNA), RNA, genomic DNA (gDNA), PCR amplified DNA, complementary DNA (cDNA), synthetic DNA, or recombinant DNA. Polynucleotides refer to a polymeric form of nucleotides of at least 5, at least 10, at least 15, at least 20, at least 25, at least 30, at least 40, at least 50, at least 100, at least 200, at least 300, at least 400, at least 500, at least 1000, at least 5000, at least 10000, or at least 15000 or more nucleotides in length, either ribonucleotides or deoxyribonucleotides or a modified form of either type of nucleotide, as well as all intermediate lengths. It will be readily understood that “intermediate lengths,” in this context, means any length between the quoted values, such as 6, 7, 8, 9, etc., 101, 102, 103, etc.; 151, 152, 153, etc.; 201, 202, 203, etc. In particular embodiments, polynucleotides or variants have at least or about 50%, 55%, 60%, 65%, 70%, 71%, 72%, 73%, 74%, 75%,76%, 77%, 78%, 79%, 80%, 81%, 82%, 83%, 84%,85%, 86%, 87%, 88%, 89%, 90%, 91%, 92%, 93%, 94%, 95%, 96%, 97%, 98%, 99% or 100% sequence identity to a reference sequence.

As used herein, “isolated polynucleotide” refers to a polynucleotide that has been purified from the sequences which flank it in a naturally occurring state, e.g., a DNA fragment that has been removed from the sequences that are normally adjacent to the fragment. In particular embodiments, an “isolated polynucleotide” also refers to a complementary DNA (cDNA), a recombinant DNA, or other polynucleotide that does not exist in nature and that has been made by the hand of man. In particular embodiments, an isolated polynucleotide is a synthetic polynucleotide, a semi-synthetic polynucleotide, or a polynucleotide obtained or derived from a recombinant source.

In various embodiments, a polynucleotide comprises an mRNA encoding a polypeptide contemplated herein. In certain embodiments, the mRNA comprises a cap, one or more nucleotides, and a poly(A) tail.

In particular embodiments, polynucleotides may be codon-optimized. As used herein, the term “codon-optimized” refers to substituting codons in a polynucleotide encoding a polypeptide in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, (x) systematic variation of codon sets for each amino acid, and/or (xi) isolated removal of spurious translation initiation sites.

As used herein, the terms “polynucleotide variant” and “variant” and the like refer to polynucleotides displaying substantial sequence identity with a reference polynucleotide sequence or polynucleotides that hybridize with a reference sequence under stringent conditions that are defined hereinafter. These terms include polynucleotides in which one or more nucleotides have been added or deleted, or replaced with different nucleotides compared to a reference polynucleotide. In this regard, it is well understood in the art that certain alterations inclusive of mutations, additions, deletions and substitutions can be made to a reference polynucleotide whereby the altered polynucleotide retains the biological function or activity of the reference polynucleotide.

Polynucleotide variants include polynucleotide fragments that encode biologically active polypeptide fragments or variants. As used herein, the term “polynucleotide fragment” refers to a polynucleotide fragment at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 150, 200, 250, 300, 350, 400, 450, 500, 550, 600, 650, 700, 750, 800, 850, 900, 950, 1000, 1100, 1200, 1300, 1400, 1500, 1600, 1700 or more nucleotides in length that encodes a polypeptide variant that retains at least 100%, at least 90%, at least 80%, at least 70%, at least 60%, at least 50%, at least 40%, at least 30%, at least 20%, at least 10%, or at least 5% of the naturally occurring polypeptide activity. Polynucleotide fragments refer to a polynucleotide that encodes a polypeptide that has an amino-terminal deletion, a carboxyl-terminal deletion, and/or an internal deletion or substitution of one or more amino acids of a naturally occurring or recombinantly-produced polypeptide.

The recitations “sequence identity” or, for example, comprising a “sequence 50% identical to.” as used herein, refer to the extent that sequences are identical on a nucleotide-by-nucleotide basis or an amino acid-by-amino acid basis over a window of comparison. Thus, a “percentage of sequence identity” may be calculated by comparing two optimally aligned sequences over the window of comparison, determining the number of positions at which the identical nucleic acid base (e.g., A, T, C, G, I) or the identical amino acid residue (e.g., Ala, Pro, Ser, Thr, Gly, Val, Leu, Ile, Phe, Tyr, Trp, Lys, Arg, His, Asp, Glu, Asn, Gln, Cys and Met) occurs in both sequences to yield the number of matched positions, dividing the number of matched positions by the total number of positions in the window of comparison (i.e., the window size), and multiplying the result by 100 to yield the percentage of sequence identity. Included are nucleotides and polypeptides having at least about 50%, 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 86%, 97%, 98%, or 99% sequence identity to any of the reference sequences described herein, typically where the polypeptide variant maintains at least one biological activity of the reference polypeptide.

Terms used to describe sequence relationships between two or more polynucleotides or polypeptides include “reference sequence,” “comparison window,” “sequence identity,” “percentage of sequence identity,” and “substantial identity”. A “reference sequence” is at least 12 but frequently 15 to 18 and often at least 25 monomer units, inclusive of nucleotides and amino acid residues, in length. Because two polynucleotides may cach comprise (1) a sequence (i.e., only a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity. A “comparison window” refers to a conceptual segment of at least 6 contiguous positions, usually about 50 to about 100, more usually about 100 to about 150 in which a sequence is compared to a reference sequence of the same number of contiguous positions after the two sequences are optimally aligned. The comparison window may comprise additions or deletions (i.e., gaps) of about 20% or less as compared to the reference sequence (which does not comprise additions or deletions) for optimal alignment of the two sequences. Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res. 25:3389. A detailed discussion of sequence analysis can be found in Unit 19.3 of Ausubel et al., Current Protocols in Molecular Biology, John Wiley & Sons Inc. 1994-1998, Chapter 15.

Terms that describe the orientation of polynucleotides include: 5′ (normally the end of the polynucleotide having a free phosphate group) and 3′ (normally the end of the polynucleotide having a free hydroxyl (OH) group). Polynucleotide sequences can be annotated in the 5′ to 3′ orientation or the 3′ to 5′ orientation. For DNA and mRNA, the 5′ to 3′ strand is designated the “sense,” “plus,” or “coding” strand because its sequence is identical to the sequence of the premessenger (premRNA) [except for uracil (U) in RNA, instead of thymine (T) in DNA]. For DNA and mRNA, the complementary 3′ to 5′ strand which is the strand transcribed by the RNA polymerase is designated as “template,” “antisense,” “minus,” or “non-coding” strand. As used herein, the term “reverse orientation” refers to a 5′ to 3′ sequence written in the 3′ to 5′ orientation or a 3′ to 5′ sequence written in the 5′ to 3′ orientation.

The terms “complementary” and “complementarity” refer to polynucleotides (i.e., a sequence of nucleotides) related by the base-pairing rules. For example, the complementary strand of the DNA sequence 5′ A G T C A T G 3′ is 3′ T C A G T A C 5′. The latter sequence is often written as the reverse complement with the 5′ end on the left and the 3′ end on the right, 5′ C A T G A C T 3′. A sequence that is equal to its reverse complement is said to be a palindromic sequence. Complementarity can be “partial,” in which only some of the nucleic acids' bases are matched according to the base pairing rules. Or, there can be “complete” or “total” complementarity between the nucleic acids.

Moreover, it will be appreciated by those of ordinary skill in the art that, as a result of the degeneracy of the genetic code, there are many nucleotide sequences that encode a polypeptide, or fragment of variant thereof, as described herein. Some of these polynucleotides bear minimal homology to the nucleotide sequence of any native gene. Nonetheless, polynucleotides that vary due to differences in codon usage are specifically contemplated in particular embodiments, for example polynucleotides that are optimized for human and/or primate codon selection. Further, alleles of the genes comprising the polynucleotide sequences provided herein may also be used. Alleles are endogenous genes that are altered as a result of one or more mutations, such as deletions, additions and/or substitutions of nucleotides.

The term “nucleic acid cassette” or “expression cassette” as used herein refers to genetic sequences within the vector which can express an RNA, and subsequently a polypeptide. In one embodiment, the nucleic acid cassette contains a gene(s)-of-interest, e.g., a polynucleotide(s)-of-interest. In another embodiment, the nucleic acid cassette contains one or more expression control sequences, e.g., a promoter, enhancer, poly(A) sequence, and a gene(s)-of-interest, e.g., a polynucleotide(s)-of-interest. Vectors may comprise 1, 2, 3, 4, 5, 6, 7, 8, 9 or 10 or more nucleic acid cassettes. The nucleic acid cassette is positionally and sequentially oriented within the vector such that the nucleic acid in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular compartments. Preferably, the cassette has its 3′ and 5′ ends adapted for ready insertion into a vector, e.g., it has restriction endonuclease sites at each end. In a preferred embodiment, the nucleic acid cassette encodes a CAR and/or CCR. The cassette can be removed and inserted into a plasmid or viral vector as a single unit.

Polynucleotides include polynucleotide(s)-of-interest. As used herein, the term “polynucleotide-of-interest” refers to a polynucleotide encoding a polypeptide, polypeptide variant, or fusion polypeptide. A vector may comprise 1, 2, 3, 4, 5, 6, 7, 8, 9, or 10 polynucleotides-of-interest. In certain embodiments, the polynucleotide-of-interest encodes a polypeptide that provides a therapeutic effect in the treatment or prevention of a disease or disorder. Polynucleotides-of-interest, and polypeptides encoded therefrom, include both polynucleotides that encode wild-type polypeptides, as well as functional variants and fragments thereof. In particular embodiments, a functional variant has at least 80%, at least 90%, at least 95%, or at least 99% identity to a corresponding wild-type reference polynucleotide or polypeptide sequence. In certain embodiments, a functional variant or fragment has at least 50%, at least 60%, at least 70%, at least 80%, or at least 90% of a biological activity of a corresponding wild-type polypeptide.

The polynucleotides contemplated herein, regardless of the length of the coding sequence itself, may be combined with other DNA sequences, such as promoters and/or enhancers, untranslated regions (UTRs), signal sequences, Kozak sequences, polyadenylation signals, additional restriction enzyme sites, multiple cloning sites, internal ribosomal entry sites (IRES), recombinase recognition sites (e.g., LoxP. FRT, and Att sites), termination codons, transcriptional termination signals, and polynucleotides encoding self-cleaving polypeptides, epitope tags, as disclosed elsewhere herein or as known in the art, such that their overall length may vary considerably. It is therefore contemplated that a polynucleotide fragment of almost any length may be employed in particular embodiments, with the total length preferably being limited by the ease of preparation and use in the intended recombinant DNA protocol.

Polynucleotides can be prepared, manipulated and/or expressed using any of a variety of well-established techniques known and available in the art. In order to express a desired polypeptide, a nucleotide sequence encoding the polypeptide, can be inserted into appropriate vector.

Illustrative examples of vectors include, but are not limited to plasmid, autonomously replicating sequences, and transposable elements, e.g., piggyBac, Sleeping Beauty, Mos1, Tc1/mariner, Tol2, mini-Tol2, Tc3, MuA, Himar I, Frog Prince, and derivatives thereof.

Additional Illustrative examples of vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses.

Illustrative examples of viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40).

Illustrative examples of expression vectors include, but are not limited to, pClneo vectors (Promega) for expression in mammalian cells; pLenti4/V5-DEST™, pLenti6/V5-DEST™, and pLenti6.2/V5-GW/lacZ (Invitrogen) for lentivirus-mediated gene transfer and expression in mammalian cells. In particular embodiments, coding sequences of polypeptides disclosed herein can be ligated into such expression vectors for the expression of the polypeptides in mammalian cells.

In particular embodiments, the vector is an episomal vector or a vector that is maintained extrachromosomally. As used herein, the term “episomal” refers to a vector that is able to replicate without integration into host's chromosomal DNA and without gradual loss from a dividing host cell also meaning that said vector replicates extrachromosomally or episomally.

The “control elements” or “regulatory sequences” present in an expression vector are those non-translated regions of the vector—origin of replication, selection cassettes, promoters, enhancers, translation initiation signals (Shine Dalgarno sequence or Kozak sequence) introns, a polyadenylation sequence, 5′ and 3′ untranslated regions—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

In particular embodiments, vectors include, but are not limited to expression vectors and viral vectors, will include exogenous, endogenous, or heterologous control sequences such as promoters and/or enhancers. An “endogenous” control sequence is one which is naturally linked with a given gene in the genome. An “exogenous” control sequence is one which is placed in juxtaposition to a gene by means of genetic manipulation (i.e., molecular biological techniques) such that transcription of that gene is directed by the linked enhancer/promoter. A “heterologous” control sequence is an exogenous sequence that is from a different species than the cell being genetically manipulated.

The term “promoter” as used herein refers to a recognition site of a polynucleotide (DNA or RNA) to which an RNA polymerase binds. An RNA polymerase initiates and transcribes polynucleotides operably linked to the promoter. In particular embodiments, promoters operative in mammalian cells comprise an AT-rich region located approximately 25 to 30 bases upstream from the site where transcription is initiated and/or another sequence found 70 to 80 bases upstream from the start of transcription, a CNCAAT region where N may be any nucleotide.

The term “enhancer” refers to a segment of DNA which contains sequences capable of providing enhanced transcription and in some instances can function independent of their orientation relative to another control sequence. An enhancer can function cooperatively or additively with promoters and/or other enhancer elements. The term “promoter/enhancer” refers to a segment of DNA which contains sequences capable of providing both promoter and enhancer functions.

The term “operably linked”, refers to a juxtaposition wherein the components described are in a relationship permitting them to function in their intended manner. In one embodiment, the term refers to a functional linkage between a nucleic acid expression control sequence (such as a promoter, and/or enhancer) and a second polynucleotide sequence, e.g., a polynucleotide-of-interest, wherein the expression control sequence directs transcription of the nucleic acid corresponding to the second sequence.

As used herein, the term “constitutive expression control sequence” refers to a promoter, enhancer, or promoter/enhancer that continually or continuously allows for transcription of an operably linked sequence. A constitutive expression control sequence may be a “ubiquitous” promoter, enhancer, or promoter/enhancer that allows expression in a wide variety of cell and tissue types or a “cell specific,” “cell type specific,” “cell lineage specific,” or “tissue specific” promoter, enhancer, or promoter/enhancer that allows expression in a restricted variety of cell and tissue types, respectively.

Illustrative ubiquitous expression control sequences suitable for use in particular embodiments include, but are not limited to, a cytomegalovirus (CMV) immediate early promoter, a viral simian virus 40 (SV40) (e.g., early or late), a Moloney murine leukemia virus (MoMLV) LTR promoter, a Rous sarcoma virus (RSV) LTR, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus (Irions et al., Nature Biotechnology 25, 1477 - 1482 (2007)), a Ubiquitin C promoter (UBC), a phosphoglycerate kinase-1 (PGK) promoter, a cytomegalovirus enhancer/chicken β-actin (CAG) promoter, a β-actin promoter and a myeloproliferative sarcoma virus enhancer, negative control region deleted, d1587rev primer-binding site substituted (MND) U3 promoter (Haas et al. Journal of Virology. 2003;77(17): 9439-9450).

In one embodiment, a vector comprises an MNDU3 promoter.

In one embodiment, a vector comprises an EF1a promoter comprising the first intron of the human EF1a gene.

In one embodiment, a vector comprises an EF1a promoter that lacks the first intron of the human EF1a gene.

As used herein, “conditional expression” may refer to any type of conditional expression including, but not limited to, inducible expression; repressible expression; expression in cells or tissues having a particular physiological, biological, or disease state, etc. This definition is not intended to exclude cell type or tissue specific expression. Certain embodiments provide conditional expression of a polynucleotide-of-interest, e.g., expression is controlled by subjecting a cell, tissue, organism, etc., to a treatment or condition that causes the polynucleotide to be expressed or that causes an increase or decrease in expression of the polynucleotide encoded by the polynucleotide-of-interest.

Illustrative examples of inducible promoters/systems include, but are not limited to, steroid-inducible promoters such as promoters for genes encoding glucocorticoid or estrogen receptors (inducible by treatment with the corresponding hormone), metallothionine promoter (inducible by treatment with various heavy metals), MX-1 promoter (inducible by interferon), the “GeneSwitch” mifepristone-regulatable system (Sirin et al., 2003, Gene, 323:67), the cumate inducible gene switch (WO 2002/088346), tetracycline-dependent regulatory systems, etc.

Conditional expression can also be achieved by using a site-specific DNA recombinase. According to certain embodiments the vector comprises at least one (typically two) site(s) for recombination mediated by a site-specific recombinase. As used herein, the terms “recombinase” or “site specific recombinase” include excisive or integrative proteins, enzymes, co-factors or associated proteins that are involved in recombination reactions involving one or more recombination sites (e.g., two, three, four, five, seven, ten, twelve, fifteen, twenty, thirty, fifty, etc.), which may be wild-type proteins (see Landy, Current Opinion in Biotechnology 3:699-707 (1993)), or mutants, derivatives (e.g., fusion proteins containing the recombination protein sequences or fragments thereof), fragments, and variants thereof. Illustrative examples of recombinases suitable for use in particular embodiments include, but are not limited to: Cre, Int, IHF, Xis, Flp, Fis, Hin, Gin, ΦC31, Cin, Tn3 resolvase, TndX, XerC, XerD, TnpX, Hjc, Gin, SpCCE1, and ParA.

The vectors may comprise one or more recombination sites for any of a wide variety of site-specific recombinases. It is to be understood that the target site for a site-specific recombinase is in addition to any site(s) required for integration of a vector, e.g., a retroviral vector or lentiviral vector. As used herein, the terms “recombination sequence.” “recombination site.” or “site specific recombination site” refer to a particular nucleic acid sequence to which a recombinase recognizes and binds.

For example, one recombination site for Cre recombinase is loxP which is a 34 base pair sequence comprising two 13 base pair inverted repeats (serving as the recombinase binding sites) flanking an 8 base pair core sequence (see FIG. 1 of Sauer, B., Current Opinion in Biotechnology 5:521-527 (1994)). Other exemplary loxP sites include, but are not limited to: lox511 (Hoess et al., 1996; Bethke and Sauer, 1997), lox5171 (Lec and Saito, 1998), lox2272 (Lee and Saito, 1998), m2 (Langer et al., 2002), lox71 (Albert et al., 1995), and lox66 (Albert et al., 1995).

Suitable recognition sites for the FLP recombinase include, but are not limited to: FRT (McLeod, et al., 1996), F₁, F₂, F₃ (Schlake and Bode, 1994), F₄, F₅ (Schlake and Bode, 1994), FRT(LE) (Senecoff et al., 1988), FRT(RE) (Senecoff et al., 1988).

Other examples of recognition sequences are the attB, attP, attL, and attR sequences, which are recognized by the recombinase enzyme λ Integrase, e.g., phi-c31. The φC31 SSR mediates recombination only between the heterotypic sites attB (34 bp in length) and attP (39 bp in length) (Groth et al., 2000). attB and attP, named for the attachment sites for the phage integrase on the bacterial and phage genomes, respectively, both contain imperfect inverted repeats that are likely bound by φC31 homodimers (Groth et al., 2000). The product sites, attL and attR, are effectively inert to further φC31-mediated recombination (Belteki et al., 2003), making the reaction irreversible. For catalyzing insertions, it has been found that attB-bearing DNA inserts into a genomic attP site more readily than an attP site into a genomic attB site (Thyagarajan et al., 2001; Belteki et al., 2003). Thus, typical strategies position by homologous recombination an attP-bearing “docking site” into a defined locus, which is then partnered with an attB-bearing incoming sequence for insertion.

As used herein, an “internal ribosome entry site” or “IRES” refers to an element that promotes direct internal ribosome entry to the initiation codon, such as ATG, of a cistron (a protein encoding region), thereby leading to the cap-independent translation of the gene. See, e.g., Jackson et al., 1990. Trends Biochem Sci 15(12):477-83) and Jackson and Kaminski. 1995. RNA 1(10):985-1000. In particular embodiments, vectors include one or more polynucleotides-of-interest that encode one or more polypeptides. In particular embodiments, to achieve efficient translation of each of the plurality of polypeptides, the polynucleotide sequences can be separated by one or more IRES sequences or polynucleotide sequences encoding self-cleaving polypeptides. In one embodiment, the IRES used in polynucleotides contemplated herein is an EMCV IRES.

As used herein, the term “Kozak sequence” refers to a short nucleotide sequence that greatly facilitates the initial binding of mRNA to the small subunit of the ribosome and increases translation. The consensus Kozak sequence is (GCC)RCCATGG (SEQ ID NO: 57), where R is a purine (A or G) (Kozak, 1986. Cell. 44(2):283-92, and Kozak, 1987. Nucleic Acids Res. 15(20):8125-48). In particular embodiments, the vectors comprise polynucleotides that have a consensus Kozak sequence and that encode a desired polypeptide, e.g., a CAR.

Elements directing the efficient termination and polyadenylation of the heterologous nucleic acid transcripts increases heterologous gene expression. Transcription termination signals are generally found downstream of the polyadenylation signal. In particular embodiments, vectors comprise a polyadenylation sequence 3′ of a polynucleotide encoding a polypeptide to be expressed. The term “polyA site” or “polyA sequence” as used herein denotes a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Cleavage and polyadenylation is directed by a poly(A) sequence in the RNA. The core poly(A) sequence for mammalian pre-mRNAs has two recognition elements flanking a cleavage-polyadenylation site. Typically, an almost invariant AAUAAA hexamer lies 20-50 nucleotides upstream of a more variable element rich in U or GU residues. Cleavage of the nascent transcript occurs between these two elements and is coupled to the addition of up to 250 adenosines to the 5′ cleavage product. In particular embodiments, the core poly(A) sequence is an ideal polyA sequence (e.g., AATAAA, ATTAAA, AGTAAA). In particular embodiments, the poly(A) sequence is an SV40 polyA sequence, a bovine growth hormone polyA sequence (BGHpA), a rabbit β-globin polyA sequence (rβgpA), variants thereof, or another suitable heterologous or endogenous polyA sequence known in the art.

In some embodiments, a polynucleotide or cell harboring the polynucleotide utilizes a suicide gene, including an inducible suicide gene to reduce the risk of direct toxicity and/or uncontrolled proliferation. In specific aspects, the suicide gene is not immunogenic to the host harboring the polynucleotide or cell. A certain example of a suicide gene that may be used is caspase-9 or caspase-8 or cytosine deaminase. Caspase-9 can be activated using a specific chemical inducer of dimerization (CID).

### G. Vectors

In certain embodiments, one or more polynucleotides encoding a CAR and a CCR are introduced into a cell (e.g., an immune effector cell) by non-viral or viral vectors. In particular embodiments, a polycistronic polynucleotide encoding a CAR and a CCR is introduced into a cell by a non-viral or viral vector. In particular embodiments, a polycistronic polynucleotide encoding a fusion protein encoding a CAR, a 2A self-cleaving polypeptide, and a CCR is introduced into a cell by a non-viral or viral vector.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, non-viral vectors are used to deliver one or more polynucleotides contemplated herein to a T cell. In one embodiment, the vector is an in vitro synthesized or synthetically prepared mRNA encoding a polycistronic message encoding a CAR and a CCR.

In one embodiment, the vector is an in vitro synthesized or synthetically prepared mRNA encoding a fusion protein encoding a CAR, a 2A self-cleaving polypeptide, and a CCR.

Illustrative examples of non-viral vectors include, but are not limited to mRNA, plasmids (e.g., DNA plasmids or RNA plasmids), transposons, cosmids, and bacterial artificial chromosomes.

Illustrative methods of non-viral delivery of polynucleotides or vectors contemplated in particular embodiments include, but are not limited to: electroporation, sonoporation, lipofection, microinjection, biolistics, virosomes, liposomes, immunoliposomes, nanoparticles, polycation or lipid:nucleic acid conjugates, naked DNA, artificial virions, DEAE-dextran-mediated transfer, gene gun, and heat-shock.

Illustrative examples of polynucleotide delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

In various embodiments, the polynucleotide is an mRNA that is introduced into a cell in order to transiently express a desired polypeptide. As used herein, “transient” refers to expression of a non-integrated transgene for a period of hours, days or weeks, wherein the period of time of expression is less than the period of time for expression of the polynucleotide if integrated into the genome or contained within a stable plasmid replicon in the cell.

In particular embodiments, the mRNA encoding a polypeptide is an in vitro transcribed mRNA. As used herein, “in vitro transcribed RNA” refers to RNA, preferably mRNA that has been synthesized in vitro. Generally, the in vitro transcribed RNA is generated from an in vitro transcription vector. The in vitro transcription vector comprises a template that is used to generate the in vitro transcribed RNA.

In particular embodiments, mRNAs may further comprise a comprise a 5′ cap or modified 5′ cap and/or a poly(A) sequence. As used herein, a 5′ cap (also termed an RNA cap, an RNA 7-methylguanosine cap or an RNA m^{7G} cap) is a modified guanine nucleotide that has been added to the “front” or 5′ end of a eukaryotic messenger RNA shortly after the start of transcription. The 5′ cap comprises a terminal group which is linked to the first transcribed nucleotide and recognized by the ribosome and protected from RNases. The capping moiety can be modified to modulate functionality of mRNA such as its stability or efficiency of translation. In a particular embodiment, the mRNA comprises a poly(A) sequence of between about 50 and about 5000 adenines. In one embodiment, the mRNA comprises a poly(A) sequence of between about 100 and about 1000 bases, between about 200 and about 500 bases, or between about 300 and about 400 bases. In one embodiment, the mRNA comprises a poly(A) sequence of about 65 bases, about 100 bases, about 200 bases, about 300 bases, about 400 bases, about 500 bases, about 600 bases, about 700 bases, about 800 bases, about 900 bases, or about 1000 or more bases. poly(A) sequences can be modified chemically or enzymatically to modulate mRNA functionality such as localization, stability or efficiency of translation.

Viral vectors comprising polynucleotides contemplated in particular embodiments can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., mobilized peripheral blood, lymphocytes, bone marrow aspirates, tissue biopsy, etc.) or universal donor hematopoietic stem cells, followed by reimplantation of the cells into a patient.

In one embodiment, a viral vector comprising a polynucleotide encoding an anti- CAR and a CCR is administered directly to an organism for transduction of cells in vivo. In one embodiment, a viral vector comprising a polynucleotide encoding a CAR, a 2A self-cleaving polypeptide, and a CCR is administered directly to an organism for transduction of cells in vivo. Alternatively, naked DNA can be administered. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides encoding a polycistronic message encoding a CAR and a CCR or a fusion protein encoding a CAR, a 2A self-cleaving polypeptide, and a CCR are introduced into an immune effector cell, e.g., a T cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides encoding a polycistronic message encoding a CAR and a CCR or a fusion protein encoding a CAR, a 2A self-cleaving polypeptide, and a CCR are introduced into an immune effector cell, by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L, Q221 L, W235F, W235E, K236S, K236A, K246A, G247W, D253A, R262A, R263A and K264H.

In one embodiment, the HIV-1 integrase deficient pol gene comprises a D64V, D116I, D116A, E152G, or E152A mutation; D64V, D116I, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., carly or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus that has viral envelope proteins that have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009;9:10. doi: 10.1186/1472-6750-9-10; Kutner et al. Nat. Protoc. 2009;4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments, one or more polynucleotides encoding a polycistronic message encoding a CAR and a CCR or a fusion protein encoding a CAR, a 2A self-cleaving polypeptide, and a CCR are introduced into an immune effector cell by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides encoding a polycistronic message encoding a CAR and a CCR or a fusion protein encoding a CAR, a 2A self-cleaving polypeptide, and a CCR are introduced into an immune effector cell by transducing the cell with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, carly, or late HSV genes to prevent replication. For example, the HSV vector may be deficient in an immediate carly gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804.413, and International Patent Applications WO 91/02788, WO 96/04394, WO 98/15637, and WO 99/06583, each of which are incorporated by reference herein in its entirety.

### H. Genetically Modified Cells

In various embodiments, cells genetically modified to express a CAR and a CCR contemplated herein, for use in the treatment of cancer are provided. As used herein, the term “genetically engineered” or “genetically modified” refers to the addition of extra genetic material in the form of DNA or RNA into the total genetic material in a cell. The terms, “genetically modified cells,” “modified cells,” and, “redirected cells,” are used interchangeably. As used herein, the term “gene therapy” refers to the introduction of extra genetic material in the form of DNA or RNA into the total genetic material in a cell that restores, corrects, or modifies expression of a gene, or for the purpose of expressing an a CAR and a CCR or a fusion protein encoding a CAR, a 2A self-cleaving polypeptide, and a CCR.

In particular embodiments, a CAR and a CCR or a fusion protein encoding a CAR, a 2A self-cleaving polypeptide, and a CCR contemplated herein are introduced and expressed in immune effector cells so as to redirect their specificity to a target antigen of interest. An “immune effector cell,” is any cell of the immune system that has one or more effector functions (e.g., cytotoxic cell killing activity, secretion of cytokines, induction of ADCC and/or CDC). Illustrative immune effector cells contemplated herein are T lymphocytes, including but not limited to cytotoxic T cells (CTLs; CD8+ T cells), TILs, and helper T cells (HTLs; CD4+ T cells). In a particular embodiment, the cells comprise αβ T cells. In a particular embodiment, the cells comprise γδ T cells. In one embodiment, immune effector cells include natural killer (NK) cells. In one embodiment, immune effector cells include natural killer T (NKT) cells.

Immune effector cells can be autologous/autogeneic (“self”) or non-autologous (“non-self,” e.g., allogeneic, syngeneic or xenogeneic). “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In preferred embodiments, the cells are autologous.

Illustrative immune effector cells used with the CARs and CCRs contemplated in particular embodiments include T lymphocytes. The terms “T cell” or “T lymphocyte” are art-recognized and are intended to include thymocytes, immature T lymphocytes, mature T lymphocytes, resting T lymphocytes, or activated T lymphocytes. A T cell can be a T helper (Th) cell, for example a T helper 1 (Th1) or a T helper 2 (Th2) cell. The T cell can be a helper T cell (HTL; CD4+ T cell) CD4+ T cell, a cytotoxic T cell (CTL; CD8+ T cell), CD4+CD8+ T cell, CD4−CD8− T cell, or any other subset of T cells. Other illustrative populations of T cells suitable for use in particular embodiments include naïve T cells (TN), T memory stem cells (TSCM), central memory T cells (TCM), effector memory T cells (TEM), and effector T cells (TEFF).

As would be understood by the skilled person, other cells may also be used as immune effector cells with the CARs and CCRs contemplated herein. In particular, immune effector cells also include NK cells, NKT cells, neutrophils, and macrophages. Immune effector cells also include progenitors of effector cells wherein such progenitor cells can be induced to differentiate into an immune effector cells in vivo or in vitro. Thus, in particular embodiments, immune effector cell includes progenitors of immune effectors cells such as hematopoictic stem cells (HSCs) contained within the CD34+ population of cells derived from cord blood, bone marrow or mobilized peripheral blood which upon administration in a subject differentiate into mature immune effector cells, or which can be induced in vitro to differentiate into mature immune effector cells.

The term, “CD34+ cell,” as used herein refers to a cell expressing the CD34 protein on its cell surface. “CD34,” as used herein refers to a cell surface glycoprotein (e.g., sialomucin protein) that often acts as a cell-cell adhesion factor and is involved in T cell entrance into lymph nodes. The CD34+ cell population contains hematopoietic stem cells (HSC), which upon administration to a patient differentiate and contribute to all hematopoietic lineages, including T cells, NK cells, NKT cells, neutrophils and cells of the monocyte/macrophage lineage.

Methods for making the immune effector cells that express a CAR and a CCR contemplated herein are provided in particular embodiments. In one embodiment, the method comprises transfecting or transducing immune effector cells isolated from an individual such that the immune effector cells express a polycistronic message encoding an a CAR and a CCR or a fusion protein encoding a CAR, a 2A self-cleaving polypeptide, and an a CCR contemplated herein. In certain embodiments, the immune effector cells are isolated from an individual and genetically modified without further manipulation in vitro. Such cells can then be directly re-administered into the individual. In further embodiments, the immune effector cells are first activated and stimulated to proliferate in vitro prior to being genetically modified to express a CAR and a CCR. In this regard, the immune effector cells may be cultured before and/or after being genetically modified (i.e., transduced or transfected to express a CAR and a CCR contemplated herein).

In particular embodiments, prior to in vitro manipulation or genetic modification of the immune effector cells described herein, the source of cells is obtained from a subject. In particular embodiments, modified immune effector cells comprise T cells.

In particular embodiments, PBMCs may be directly genetically modified to express a polycistronic message encoding a CAR and a CCR or a fusion protein encoding a CAR, a 2A self-cleaving polypeptide, and a CCR using methods contemplated herein. In certain embodiments, after isolation of PBMC, T lymphocytes are further isolated and in certain embodiments, both cytotoxic and helper T lymphocytes can be sorted into naïve, memory, and effector T cell subpopulations either before or after genetic modification and/or expansion.

The immune effector cells, such as T cells, can be genetically modified following isolation using known methods, or the immune effector cells can be activated and expanded (or differentiated in the case of progenitors) in vitro prior to being genetically modified. In a particular embodiment, the immune effector cells, such as T cells, are genetically modified with the chimeric antigen receptors contemplated herein (e.g., transduced with a viral vector comprising a nucleic acid encoding a polycistronic message encoding a CAR and a CCR or a fusion protein encoding a CAR, a 2A self-cleaving polypeptide, and a CCR) and then are activated and expanded in vitro. In various embodiments, T cells can be activated and expanded before or after genetic modification, using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

In one embodiment, CD34+ cells are transduced with a nucleic acid construct contemplated herein. In certain embodiments, the transduced CD34+ cells differentiate into mature immune effector cells in vivo following administration into a subject, generally the subject from whom the cells were originally isolated. In another embodiment, CD34+ cells may be stimulated in vitro prior to exposure to or after being genetically modified with one or more of the following cytokines: Flt-3 ligand (FLT3), stem cell factor (SCF), megakaryocyte growth and differentiation factor (TPO), IL-3 and IL-6 according to the methods described previously (Asheuer et al., 2004; Imren, et al., 2004).

In particular embodiments, a population of modified immune effector cells for the treatment of cancer comprises a CAR and CCR contemplated herein. For example, a population of modified immune effector cells are prepared from peripheral blood mononuclear cells (PBMCs) obtained from a patient diagnosed with B cell malignancy described herein (autologous donors). The PBMCs form a heterogeneous population of T lymphocytes that can be CD4+, CD8+, or CD4+ and CD8+.

The PBMCs also can include other cytotoxic lymphocytes such as NK cells or NKT cells. An expression vector carrying the coding sequence of a CAR and CCR contemplated in particular embodiments is introduced into a population of human donor T cells, NK cells or NKT cells. In particular embodiments, successfully transduced T cells that carry the expression vector can be sorted using flow cytometry to isolate CD3 positive T cells and then further propagated to increase the number of these CAR and CCR expressing T cells in addition to cell activation using anti-CD3 antibodies and or anti-CD28 antibodies and IL-2 or any other methods known in the art as described elsewhere herein. Standard procedures are used for cryopreservation of T cells for storage and/or preparation for use in a human subject. In one embodiment, the in vitro transduction, culture and/or expansion of T cells are performed in the absence of non-human animal derived products such as fetal calf serum and fetal bovine serum. Since a heterogeneous population of PBMCs is genetically modified, the resultant transduced cells are a heterogeneous population of modified cells comprising a polycistronic message encoding a CAR and a CCR or a polynucleotide encoding a fusion protein encoding a CAR, a 2A self-cleaving polypeptide, and a CCR as contemplated herein.

In a further embodiment, a mixture of, e.g., one, two, three, four, five or more, different expression vectors can be used in genetically modifying a donor population of immune effector cells wherein each vector encodes a different chimeric antigen receptor protein as contemplated herein. The resulting modified immune effector cells forms a mixed population of modified cells.

### I. T-Cell Manufacturing Methods

In various embodiments, genetically modified T cells are expanded by contact with an agent that stimulates a CD3 TCR complex associated signal and a ligand that stimulates a co-stimulatory molecule on the surface of the T cells.

In particular embodiments, PBMCs or isolated T cells are contacted with a stimulatory agent and co-stimulatory agent, such as soluble anti-CD3 and anti-CD28 antibodies, or antibodies attached to a bead or other surface, in a culture medium with appropriate cytokines, such as IL-2, IL-7, and/or IL-15.

In particular embodiments, PBMCs or isolated T cells are contacted with a stimulatory agent and co-stimulatory agent, such as soluble anti-CD3 and anti-CD28 antibodies, or antibodies attached to a bead or other surface, in a culture medium with appropriate cytokines, such as IL-2, IL-7, and/or IL-15 and/or a PI3K inhibitor.

In one embodiment, peripheral blood mononuclear cells (PBMCs) are used as the source of T cells in the T cell manufacturing methods contemplated herein. PBMCs form a heterogeneous population of T lymphocytes that can be CD4+, CD8+, or CD4+ and CD8+ and can include other mononuclear cells such as monocytes, B cells, NK cells and NKT cells. An expression vector comprising a polynucleotide encoding a polycistronic message encoding an CAR and a CCR or a fusion protein encoding a CAR, a 2A self-cleaving polypeptide, and a CCR contemplated in particular embodiments are introduced into a population of human donor T cells, NK cells or NKT cells. In a particular embodiment, successfully transduced T cells that carry the expression vector can be sorted using flow cytometry to isolate CD3 positive T cells and then further propagated to increase the number of the modified T cells in addition to cell activation using anti-CD3 antibodies and or anti-CD28 antibodies and IL-2, IL-7, and/or IL-15.

In order to achieve sufficient therapeutic doses of T cell compositions, T cells are often subject to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887.466; 6,905,681; 7,144,575; 7,067,318; 7,172,869; 7,232.566; 7,175,843; 5,883,223; 6,905,874; 6,797,514; and 6,867,041, each of which is incorporated herein by reference in its entirety.

In preferred embodiments, the T cells manufactured by the methods contemplated herein provide improved adoptive immunotherapy compositions. Without wishing to be bound to any particular theory, it is believed that the T cell compositions manufactured by the methods in particular embodiments contemplated herein are imbued with superior properties, including increased survival, expansion in the relative absence of differentiation, and persistence in vivo. In one embodiment, a method of manufacturing T cells comprises contacting the cells with one or more agents that modulate a PI3K cell signaling pathway.

In a particular embodiment. T cells are manufactured by stimulating T cells to become activated and to proliferate in the presence of one or more stimulatory signals and optionally, a PI3K inhibitor.

The T cells can then be modified to express a polycistronic message a CAR and a CCR or a fusion protein encoding a CAR, a 2A self-cleaving polypeptide, and a CCR. In one embodiment, the T cells are modified by transducing the T cells with a viral vector comprising a polycistronic message encoding a CAR and a CCR or a fusion protein encoding a CAR, a 2A self-cleaving polypeptide, and a CCR contemplated herein. In a certain embodiment, the T cells are modified prior to stimulation and activation. In another embodiment. T cells are modified after stimulation and activation. In a particular embodiment, T cells are modified within 12 hours, 24 hours, 36 hours, or 48 hours of stimulation and activation. In particular embodiments, the T cells are activated, stimulated, and /or modified in the presence or absence of a PI3K inhibitor.

After T cells are transduced, the cells are cultured to proliferate. T cells may be cultured for at least 1, 2, 3, 4, 5, 6, or 7 days, at least 2 weeks, at least 1, 2, 3, 4, 5, or 6 months or more with 1, 2, 3, 4, 5, 6, 7, 8, 9, or 10 or more rounds of expansion. In a particular embodiment, the T cells are cultured to proliferate.

In various embodiments, T cell compositions are manufactured in the presence of a PI3K inhibitor. Without wishing to be bound to any particular theory, it is contemplated that treatment or contacting T cells with one or more inhibitors of the PI3K pathway during any one of, any combination of, or all of, the stimulation, activation, and/or expansion phases of the manufacturing process preferentially increases young T cells, thereby producing superior therapeutic T cell compositions.

As used herein, the term “PI3K inhibitor” refers to a nucleic acid, peptide, compound, or small organic molecule that binds to and inhibits at least one activity of PI3K. The PI3K proteins can be divided into three classes, class 1 PI3Ks, class 2 PI3Ks, and class 3 PI3Ks. Class 1 PI3Ks exist as heterodimers consisting of one of four p110 catalytic subunits (p110α, p110β, p110δ, and p110γ) and one of two families of regulatory subunits. A PI3K inhibitor preferably targets the class 1 PI3K inhibitors. In one embodiment, a PI3K inhibitor will display selectivity for one or more isoforms of the class 1 PI3K inhibitors (i.e., selectivity for p110α, p110β, p110δ, and p110γ or one or more of p110α, p110β, p110δ, and p110γ). In another aspect, a PI3K inhibitor will not display isoform selectivity and be considered a “pan-PI3K inhibitor.” In one embodiment, a PI3K inhibitor will compete for binding with ATP to the PI3K catalytic domain.

In certain embodiments, a PI3K inhibitor can, for example, target PI3K as well as additional proteins in the PI3K-AKT-mTOR pathway. In particular embodiments, a PI3K inhibitor that targets both mTOR and PI3K can be referred to as either an mTOR inhibitor or a PI3K inhibitor. A PI3K inhibitor that only targets PI3K can be referred to as a selective PI3K inhibitor. In one embodiment, a selective PI3K inhibitor can be understood to refer to an agent that exhibits a 50% inhibitory concentration with respect to PI3K that is at least 10-fold, at least 20-fold, at least 30-fold, at least 50-fold, at least 100-fold, at least 1000-fold, or more, lower than the inhibitor's IC50 with respect to mTOR and/or other proteins in the pathway.

In a particular embodiment, exemplary PI3K inhibitors inhibit PI3K with an IC50 (concentration that inhibits 50% of the activity) of about 200 nM or less, preferably about 100 nm or less, even more preferably about 60 nM or less, about 25 nM, about 10 nM, about 5 nM, about 1 nM, 100 μM, 50 μM, 25 μM, 10 μM, 1 μM, or less. In one embodiment, a PI3K inhibitor inhibits PI3K with an IC50 from about 2 nM to about 100 nm, more preferably from about 2 nM to about 50 nM, even more preferably from about 2 nM to about 15 nM.

Illustrative examples of PI3K inhibitors suitable for use in the T cell manufacturing methods contemplated in particular embodiments include, but are not limited to, BKM120 (class 1 PI3K inhibitor, Novartis), XL147 (class 1 PI3K inhibitor, Exelixis), (pan-PI3K inhibitor, GlaxoSmithKline), and PX-866 (class 1 PI3K inhibitor; p110α, p110β, and p110γ isoforms, Oncothyreon).

Other illustrative examples of selective PI3K inhibitors include, but are not limited to BYL719, GSK2636771, TGX-221, AS25242, CAL-101, ZSTK474, and IPI-145.

Further illustrative examples of pan-PI3K inhibitors include, but are not limited to BEZ235, LY294002, GSK1059615, TG100713, and GDC-0941.

In a preferred embodiment, the PI3K inhibitor is ZSTK474.

In a particular embodiment, a method for increasing the proliferation of T cells expressing an engineered T cell receptor is provided. Such methods may comprise, for example, harvesting a source of T cells from a subject, stimulating and activating the T cells, modification of the T cells to express a CAR and a CCR, and expanding the T cells in culture wherein the T cells are manufactured in the presence of one or more PI3K inhibitors in any one of more steps of the manufacturing process.

Manufacturing methods contemplated herein may further comprise cryopreservation of modified T cells for storage and/or preparation for use in a human subject. In one embodiment, a method of storing genetically modified immune effector cells comprises cryopreserving the immune effector cells such that the cells remain viable upon thawing. T cells are cryopreserved such that the cells remain viable upon thawing. When needed, the cryopreserved transformed immune effector cells can be thawed, grown and expanded for more such cells. As used herein, “cryopreserving.” refers to the preservation of cells by cooling to sub-zero temperatures, such as (typically) 77 K or −196° C. (the boiling point of liquid nitrogen). Cryoprotective agents are often used at sub-zero temperatures to prevent the cells being preserved from damage due to freezing at low temperatures or warming to room temperature. Cryopreservative agents and optimal cooling rates can protect against cell injury. Cryoprotective agents which can be used include but are not limited to dimethyl sulfoxide (DMSO) (Lovelock and Bishop, Nature, 1959; 183: 1394-1395; Ashwood-Smith, Nature, 1961; 190: 1204-1205), glycerol, polyvinylpyrrolidine (Rinfret, Ann. N.Y. Acad. Sci., 1960; 85: 576), and polyethylene glycol (Sloviter and Ravdin, Nature, 1962; 196: 48). The preferred cooling rate is 1° to 3° C./minute. After at least two hours, the T cells have reached a temperature of −80° C. and can be placed directly into liquid nitrogen (−196° C.) for permanent storage such as in a long-term cryogenic storage vessel.

### J. Compositions and Formulations

In particular embodiments, formulation of pharmaceutically-acceptable carrier solutions is well-known to those of skill in the art, as is the development of suitable dosing and treatment regimens for using the particular compositions described herein in a variety of treatment regimens, including e.g., enteral and parenteral, e.g., intravascular, intravenous, intrarterial, intraosscously, intraventricular, intracerebral, intracranial, intraspinal, intrathecal, and intramedullary administration and formulation. It would be understood by the skilled artisan that particular embodiments contemplated herein may comprise other formulations, such as those that are well known in the pharmaceutical art, and are described, for example, in Remington: The Science and Practice of Pharmacy, volume I and volume II. 22nd Edition. Edited by Loyd V. Allen Jr. Philadelphia, PA: Pharmaceutical Press; 2012, which is incorporated by reference herein, in its entirety.

The compositions contemplated hercin may comprise one or more CAR polypeptides, CCR polypeptides, polynucleotides, vectors comprising same, genetically modified immune effector cells, etc., as contemplated hercin. Compositions include, but are not limited to pharmaceutical compositions. In preferred embodiments, a composition comprises one or more cells modified to express a CAR and a CCR; a fusion protein encoding a CAR, a self-cleaving polypeptide, and a CCR; or a fusion protein encoding a CAR, a self-cleaving polypeptide, and a CCR.

A “pharmaceutical composition” refers to a composition formulated in pharmaceutically-acceptable or physiologically-acceptable solutions for administration to a cell or an animal, either alone, or in combination with one or more other modalities of therapy. It will also be understood that, if desired, the compositions may be administered in combination with other agents as well, such as, e.g., cytokines, growth factors, hormones, small molecules, chemotherapeutics, pro-drugs, drugs, antibodies, or other various pharmaceutically-active agents. There is virtually no limit to other components that may also be included in the compositions, provided that the additional agents do not adversely affect the ability of the composition to deliver the intended therapy. In preferred embodiments, a pharmaceutical composition comprises a pharmaceutically acceptable carrier, diluent or excipient and one or more cells modified to express a CAR and a CCR or a fusion protein encoding a CAR and a CCR.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose and sucrose; starches, such as corn starch and potato starch; cellulose, and its derivatives, such as sodium carboxymethyl cellulose, ethyl cellulose and cellulose acetate; tragacanth; malt; gelatin; talc; cocoa butter, waxes, animal and vegetable fats, paraffins, silicones, bentonites, silicic acid, zinc oxide; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

In particular embodiments, compositions comprise an amount of CAR and CCR-expressing immune effector cells contemplated herein. As used herein, the term “amount” refers to “an amount effective” or “an effective amount” of a genetically modified therapeutic cell, e.g., T cell, to achieve a beneficial or desired prophylactic or therapeutic result, including clinical results.

A “prophylactically effective amount” refers to an amount of a genetically modified therapeutic cells effective to achieve the desired prophylactic result. Typically, but not necessarily, since a prophylactic dose is used in subjects prior to or at an earlier stage of disease, the prophylactically effective amount is less than the therapeutically effective amount.

A “therapeutically effective amount” of a genetically modified therapeutic cell may vary according to factors such as the disease state, age, sex, and weight of the individual, and the ability of the stem and progenitor cells to elicit a desired response in the individual. A therapeutically effective amount is also one in which any toxic or detrimental effects of the virus or transduced therapeutic cells are outweighed by the therapeutically beneficial effects. The term “therapeutically effective amount” includes an amount that is effective to “treat” a subject (e.g., a patient). When a therapeutic amount is indicated, the precise amount of the compositions to be administered can be determined by a physician with consideration of individual differences in age, weight, tumor size, extent of infection or metastasis, and condition of the patient (subject).

It can generally be stated that a pharmaceutical composition comprising the T cells described herein may be administered at a dosage of 102 to 1010 cells/kg body weight, preferably 105 to 106 cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 106 cells/ml and generally is greater than 107 cells/ml. generally 108 cells/ml or greater. The clinically relevant number of immune cells can be apportioned into multiple infusions that cumulatively equal or exceed 105, 106, 107, 108, 109, 1010, 1011, or 1012 cells. In some aspects, particularly since all the infused cells will be redirected to a particular target antigen, lower numbers of cells, in the range of 106/kilogram (106-1011 per patient) may be administered. Compositions may be administered multiple times at dosages within these ranges. The cells may be allogencic, syngeneic, xenogeneic, or autologous to the patient undergoing therapy. If desired, the treatment may also include administration of mitogens (e.g., PHA) or lymphokines, cytokines, and/or chemokines (e.g., IFN-γ, IL-2, IL-12, TNF-alpha, IL-18, and TNF-beta, GM-CSF, IL-4, IL-13, Flt3-L, RANTES, MIP1α, etc.) as described herein to enhance induction of the immune response.

Generally, compositions comprising the cells activated and expanded as described herein may be utilized in the treatment and prevention of diseases that arise in individuals who are immunocompromised. In particular embodiments, compositions comprising immune effector cells modified to express a CAR and a CCR or a fusion protein encoding a CAR and a CCR contemplated herein are used in the treatment of cancer. The modified immune effector cells may be administered either alone, or as a pharmaceutical composition in combination with carriers, diluents, excipients, and/or with other components such as IL-2 or other cytokines or cell populations. In particular embodiments, pharmaceutical compositions comprise an amount of genetically modified T cells, in combination with one or more pharmaceutically or physiologically acceptable carriers, diluents or excipients.

Pharmaceutical compositions comprising an immune effector cell population modified to express a CAR and a CCR or a fusion protein encoding a CAR and a CCR, such as T cells, may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions are preferably formulated for parenteral administration, e.g., intravascular (intravenous or intraarterial), intraperitoneal or intramuscular administration.

The liquid pharmaceutical compositions, whether they be solutions, suspensions or other like form, may include one or more of the following: sterile diluents such as water for injection, saline solution, preferably physiological saline, Ringer's solution, isotonic sodium chloride, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. An injectable pharmaceutical composition is preferably sterile.

In one embodiment, the T cell compositions contemplated herein are formulated in a pharmaceutically acceptable cell culture medium. Such compositions are suitable for administration to human subjects. In particular embodiments, the pharmaceutically acceptable cell culture medium is a serum free medium.

Serum-free medium has several advantages over serum containing medium, including a simplified and better-defined composition, a reduced degree of contaminants, elimination of a potential source of infectious agents, and lower cost. In various embodiments, the serum-free medium is animal-free, and may optionally be protein-free. Optionally, the medium may contain biopharmaceutically acceptable recombinant proteins. “Animal-free” medium refers to medium wherein the components are derived from non-animal sources. Recombinant proteins replace native animal proteins in animal-free medium and the nutrients are obtained from synthetic, plant or microbial sources. “Protein-free” medium, in contrast, is defined as substantially free of protein.

Illustrative examples of serum-free media used in particular compositions includes, but is not limited to QBSF-60 (Quality Biological, Inc.), StemPro-34 (Life Technologies), and X-VIVO 10.

In one preferred embodiment, compositions comprising immune effector cells contemplated herein are formulated in a solution comprising PlasmaLyte A.

In another preferred embodiment, compositions comprising immune effector cells contemplated herein are formulated in a solution comprising a cryopreservation medium. For example, cryopreservation media with cryopreservation agents may be used to maintain a high cell viability outcome post-thaw. Illustrative examples of cryopreservation media used in particular compositions includes, but is not limited to, CryoStor CS10, CryoStor CS5, and CryoStor CS2.

In a more preferred embodiment, compositions comprising immune effector cells contemplated herein are formulated in a solution comprising 50:50 PlasmaLyte A to CryoStor CS10.

In a particular embodiment, compositions comprise an effective amount of immune effector cells modified to express a CAR and a CCR or a fusion protein encoding a CAR and a CCR, alone or in combination with one or more therapeutic agents. Thus, the CAR-expressing immune effector cell compositions may be administered alone or in combination with other known cancer treatments, such as radiation therapy, chemotherapy, transplantation, immunotherapy, hormone therapy, photodynamic therapy, etc. The compositions may also be administered in combination with antibiotics. Such therapeutic agents may be accepted in the art as a standard treatment for a particular disease state as described herein, such as a particular cancer. Exemplary therapeutic agents contemplated in particular embodiments include cytokines, growth factors, steroids, NSAIDS, DMARDs, anti-inflammatoires, chemotherapeutics, radiotherapeutics, therapeutic antibodies, or other active and ancillary agents.

In certain embodiments, compositions comprising immune effector cells modified to express a CAR and a CCR or a fusion protein encoding a CAR and a CCR may be administered in conjunction with any number of chemotherapeutic agents.

A variety of other therapeutic agents may be used in conjunction with the compositions described herein. In one embodiment, the composition comprising immune effector cells a CAR and a CCR or a fusion protein encoding a CAR and a CCR is administered with an anti-inflammatory agent.

In one embodiment, the composition comprising immune effector cells a CAR and a CCR or a fusion protein encoding the same is administered with a therapeutic antibody. Illustrative examples of therapeutic antibodies suitable for combination with the CAR modified T cells contemplated in particular embodiments, include but are not limited to, atezolizumab, avelumab, bavituximab, bevacizumab (avastin), bivatuzumab, blinatumomab, conatumumab, crizotinib, daratumumab, duligotumab, dacetuzumab, dalotuzumab, durvalumab, elotuzumab (HuLuc63), gemtuzumab, ibritumomab, indatuximab, inotuzumab, ipilimumab, lorvotuzumab, lucatumumab, milatuzumab, moxetumomab, nivolumab, ocaratuzumab, ofatumumab, pembrolizumab, rituximab, siltuximab, teprotumumab, and ublituximab.

### K. Therapeutic Methods

The genetically modified immune effector cells expressing a CAR and a CCR contemplated herein provide improved methods of adoptive immunotherapy for use in the prevention, treatment, and amelioration of a cancer, or for preventing, treating, or ameliorating at least one symptom associated with a cancer.

In various embodiments, the genetically modified immune effector cells contemplated herein provide improved methods of adoptive immunotherapy for use in increasing the cytotoxicity in cancer cells in a subject or for use in decreasing the number of cancer cells in a subject.

In particular embodiments, the specificity of a primary immune effector cell is redirected to cells expressing a particular antigen, e.g., cancer cells, by genetically modifying the primary immune effector cell with a CAR and/or CCR as contemplated herein. In various embodiments, a viral vector is used to genetically modify an immune effector cell with a particular polynucleotide encoding a CAR and a CCR.

In one embodiment, a type of cellular therapy where T cells are genetically modified to express a CAR and/or a CCR that target specific antigen(s) expressing cancer cells, and the T cells are infused to a recipient in need thereof is provided. The infused cell is able to kill disease causing cells in the recipient. Unlike antibody therapies, T cell therapies are able to replicate in vivo resulting in long-term persistence that can lead to sustained cancer therapy.

In one embodiment, T cells that express a CAR and a CCR can undergo robust in vivo T cell expansion and can persist for an extended amount of time. In another embodiment, T cells that express a CAR and a CCR evolve into specific memory T cells or stem cell memory T cells that can be reactivated to inhibit any additional tumor formation or growth.

In particular embodiments, compositions comprising immune effector cells that express a CAR and a CCR contemplated herein are used in the treatment of conditions associated particular antigen-expressing cancer cells or cancer stem cells.

Illustrative examples of conditions that can be treated, prevented or ameliorated using the immune effector cells that express a CAR and a CCR are contemplated in particular embodiments

In a particular embodiment, compositions comprising T cells that express a CAR and a CCR contemplated herein are used in the treatment of osteosarcoma or Ewing's sarcoma.

In a particular embodiment, compositions comprising T cells that express a CAR and a CCR contemplated herein are used in the treatment of liquid or hematological cancers.

In certain embodiments, the liquid or hematological cancer is selected from the group consisting of: leukemias, lymphomas, and multiple myelomas.

In certain embodiments, the liquid or hematological cancer is selected from the group consisting of: acute lymphocytic leukemia (ALL), acute myeloid leukemia (AML), myeloblastic, promyelocytic, myelomonocytic, monocytic, erythroleukemia, hairy cell leukemia (HCL), chronic lymphocytic leukemia (CLL), and chronic myeloid leukemia (CML), chronic myelomonocytic leukemia (CMML) and polycythemia vera, Hodgkin lymphoma, nodular lymphocyte-predominant Hodgkin lymphoma, Burkitt lymphoma, small lymphocytic lymphoma (SLL), diffuse large B-cell lymphoma, follicular lymphoma, immunoblastic large cell lymphoma, precursor B-lymphoblastic lymphoma, mantle cell lymphoma, marginal zone lymphoma, mycosis fungoides, anaplastic large cell lymphoma, Sézary syndrome, precursor T-lymphoblastic lymphoma, multiple myeloma, overt multiple myeloma, smoldering multiple myeloma, plasma cell leukemia, non-secretory myeloma, IgD myeloma, osteosclerotic myeloma, solitary plasmacytoma of bone, and extramedullary plasmacytoma.

In certain embodiments, the liquid or hematological cancer is selected from the group consisting of: acute lymphocytic leukemia (ALL), chronic lymphocytic leukemia (CLL), hairy cell leukemia (HCL), multiple myeloma (MM), acute myeloid leukemia (AML), or chronic myeloid leukemia (CML).

In preferred embodiments, the liquid or hematological cancer is DLBCL.

In preferred embodiments, the liquid or hematological cancer is relapsed/refractory DLBCL.

In particular embodiments, methods comprising administering a therapeutically effective amount of immune effector cells that express a CAR and a CCR contemplated herein or a composition comprising the same, to a patient in need thereof, alone or in combination with one or more therapeutic agents, are provided. In certain embodiments, the cells are used in the treatment of patients at risk for developing a condition associated with cancer cells. Thus, in particular embodiments, methods for the treatment or prevention or amelioration of at least one symptom of cancer comprising administering to a subject in need thereof, a therapeutically effective amount of the modified T cells that express a CAR and a CCR contemplated herein.

As used herein, the terms “individual” and “subject” are often used interchangeably and refer to any animal that exhibits a symptom of a disease, disorder, or condition that can be treated with the gene therapy vectors, cell-based therapeutics, and methods contemplated elsewhere herein. In preferred embodiments, a subject includes any animal that exhibits symptoms of a disease, disorder, or condition related to cancer that can be treated with the gene therapy vectors, cell-based therapeutics, and methods contemplated elsewhere herein. Suitable subjects (e.g., patients) include laboratory animals (such as mouse, rat, rabbit, or guinea pig), farm animals, and domestic animals or pets (such as a cat or dog). Non-human primates and, preferably, human patients, are included. Typical subjects include human patients that have a cancer, have been diagnosed with a cancer, or are at risk or having a cancer.

As used herein, the term “patient” refers to a subject that has been diagnosed with a particular disease, disorder, or condition that can be treated with the gene therapy vectors, cell-based therapeutics, and methods disclosed elsewhere herein.

As used herein “treatment” or “treating,” includes any beneficial or desirable effect on the symptoms or pathology of a disease or pathological condition, and may include even minimal reductions in one or more measurable markers of the disease or condition being treated. Treatment can involve optionally either the reduction the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

As used herein, the phrase “ameliorating at least one symptom of” refers to decreasing one or more symptoms of the disease or condition for which the subject is being treated. In particular embodiments, the disease or condition being treated is a cancer, wherein the one or more symptoms ameliorated include, but are not limited to, weakness, fatigue, shortness of breath, casy bruising and bleeding, frequent infections, enlarged lymph nodes, distended or painful abdomen (due to enlarged abdominal organs), bone or joint pain, fractures, unplanned weight loss, poor appetite, night sweats, persistent mild fever, and decreased urination (due to impaired kidney function).

By “enhance” or “promote,” or “increase” or “expand” refers generally to the ability of a composition contemplated herein, e.g., a genetically modified T cells that express a CAR and a CCR, to produce, elicit, or cause a greater physiological response (i.e., downstream effects) compared to the response caused by either vehicle or a control molecule/composition. A measurable physiological response may include an increase in T cell expansion, activation, persistence, and/or an increase in cancer cell killing ability, among others apparent from the understanding in the art and the description herein. An “increased” or “enhanced” amount is typically a “statistically significant” amount, and may include an increase that is 1.1. 1.2. 1.5, 2, 3, 4, 5, 6, 7, 8, 9, 10, 15, 20, 30 or more times (e.g., 500, 1000 times) (including all integers and decimal points in between and above 1, e.g., 1.5. 1.6, 1.7. 1.8, etc.) the response produced by vehicle or a control composition.

By “decrease” or “lower,” or “lessen,” or “reduce,” or “abate” refers generally to the ability of composition contemplated herein to produce, elicit, or cause a lesser physiological response (i.e., downstream effects) compared to the response caused by either vehicle or a control molecule/composition. A “decrease” or “reduced” amount is typically a “statistically significant” amount, and may include an decrease that is 1.1, 1.2, 1.5, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the response (reference response) produced by vehicle, a control composition, or the response in a particular cell lineage.

By “maintain,” or “preserve,” or “maintenance,” or “no change,” or “no substantial change,” or “no substantial decrease” refers generally to the ability of a composition contemplated herein to produce, elicit, or cause a similar physiological response (i.e., downstream effects) in a cell, as compared to the response caused by either vehicle, a control molecule/composition, or the response in a particular cell lineage. A comparable response is one that is not significantly different or measurable different from the reference response.

In one embodiment, a method of treating cancer in a subject in need thereof comprises administering an effective amount, e.g., therapeutically effective amount of a composition comprising genetically modified immune effector cells contemplated herein. The quantity and frequency of administration will be determined by such factors as the condition of the patient, and the type and severity of the patient's disease, although appropriate dosages may be determined by clinical trials.

In one embodiment, the amount of immune effector cells, e.g., T cells that express a CAR and a CCR, in the composition administered to a subject is at least 0.1×10⁵ cells, at least 0.5×10⁵ cells, at least 1×10⁵ cells, at least 5×10⁵ cells, at least 1×10⁶ cells, at least 0.5×10⁷ cells, at least 1×10⁷ cells, at least 0.5×10⁸ cells, at least 1×10⁸ cells, at least 0.5×10⁹ cells, at least 1×10⁹ cells, at least 2×10⁹ cells, at least 3×10⁹ cells, at least 4×10⁹ cells, at least 5×10⁹ cells, or at least 1×10¹⁰ cells.

In particular embodiments, about 1×10⁷ T cells to about 1×10⁹ T cells, about 2×10⁷ T cells to about 0.9×10⁹ T cells, about 3×10⁷ T cells to about 0.8×10⁹ T cells, about 4×10⁷ T cells to about 0.7×10⁹ T cells, about 5×10⁷ T cells to about 0.6×10⁹ T cells, or about 5×10⁷ T cells to about 0.5×10⁹ T cells are administered to a subject.

In one embodiment, the amount of immune effector cells, e.g., T cells that express an a CAR and a CCR, in the composition administered to a subject is at least 0.1×10⁴ cells/kg of bodyweight, at least 0.5×10⁴ cells/kg of bodyweight, at least 1×10⁴ cells/kg of bodyweight, at least 5×10⁴ cells/kg of bodyweight, at least 1 33 10⁵ cells/kg of bodyweight, at least 0.5×10⁶ cells/kg of bodyweight, at least 1×10⁶ cells/kg of bodyweight, at least 0.5×10⁷ cells/kg of bodyweight, at least 1×10⁷ cells/kg of bodyweight, at least 0.5×10⁸ cells/kg of bodyweight, at least 1×10⁸ cells/kg of bodyweight, at least 2×10⁸ cells/kg of bodyweight, at least 3×10⁸ cells/kg of bodyweight, at least 4×10⁸ cells/kg of bodyweight, at least 5×10⁸ cells/kg of bodyweight, or at least 1×10⁹ cells/kg of bodyweight.

In particular embodiments, about 1×10⁶ T cells/kg of bodyweight to about 1×10⁸ T cells/kg of bodyweight, about 2×10⁶ T cells/kg of bodyweight to about 0.9×10⁸ T cells/kg of bodyweight, about 3×10⁶ T cells/kg of bodyweight to about 0.8×10⁸ T cells/kg of bodyweight, about 4×10⁶ T cells/kg of bodyweight to about 0.7×10⁸ T cells/kg of bodyweight, about 5×10⁶ T cells/kg of bodyweight to about 0.6×10⁸ T cells/kg of bodyweight, or about 5×10⁶ T cells/kg of bodyweight to about 0.5×10⁸ T cells/kg of bodyweight are administered to a subject.

One of ordinary skill in the art would recognize that multiple administrations of the compositions contemplated herein may be required to affect the desired therapy. For example a composition may be administered 1, 2, 3, 4, 5, 6, 7, 8, 9, or 10 or more times over a span of 1 weck, 2 weeks, 3 weeks, 1 month, 2 months, 3 months, 4 months, 5 months, 6 months, 1 year, 2 years, 5, years, 10 years, or more.

In certain embodiments, it may be desirable to administer activated immune effector cells to a subject and then subsequently redraw blood (or have an apheresis performed), activate immune effector cells therefrom, and reinfuse the patient with these activated and expanded immune effector cells. This process can be carried out multiple times every few weeks. In certain embodiments, immune effector cells can be activated from blood draws of from 10 cc to 400 cc. In certain embodiments, immune effector cells are activated from blood draws of 20 cc, 30 cc, 40 cc, 50 cc, 60 cc, 70 cc, 80 cc, 90 cc, 100 cc, 150 cc, 200 cc, 250 cc, 300 cc, 350 cc, or 400 cc or more. Not to be bound by theory, using this multiple blood draw/multiple reinfusion protocol may serve to select out certain populations of immune effector cells.

The administration of the compositions contemplated herein may be carried out in any convenient manner, including by aerosol inhalation, injection, ingestion, transfusion, implantation or transplantation. In a preferred embodiment, compositions are administered parenterally. The phrases “parenteral administration” and “administered parenterally” as used herein refers to modes of administration other than enteral and topical administration, usually by injection, and includes, without limitation, intravascular, intravenous, intramuscular, intraarterial, intrathecal, intracapsular, intraorbital, intratumoral, intracardiac, intradermal, intraperitoneal, transtracheal, subcutaneous, subcuticular, intraarticular, subcapsular, subarachnoid, intraspinal and intrasternal injection and infusion. In one embodiment, the compositions contemplated herein are administered to a subject by direct injection into a tumor, lymph node, or site of infection.

In one embodiment, a subject in need thereof is administered an effective amount of a composition to increase a cellular immune response to a B cell related condition in the subject. The immune response may include cellular immune responses mediated by cytotoxic T cells capable of killing infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Current Protocols in Immunology, Edited by: John E. Coligan, Ada M. Kruisbeek, David H. Margulies, Ethan M. Shevach, Warren Strober (2001) John Wiley & Sons, NY, N.Y.

In one embodiment, a method of treating a subject diagnosed with a cancer is provided comprising removing immune effector cells from the subject, genetically modifying said immune effector cells with a vector comprising a nucleic acid encoding a CAR and a CCR contemplated herein, thereby producing a population of modified immune effector cells, and administering the population of modified immune effector cells to the same subject. In a preferred embodiment, the immune effector cells comprise T cells.

In certain embodiments, methods for stimulating an immune effector cell mediated immune modulator response to a target cell population in a subject are provided comprising the steps of administering to the subject an immune effector cell population expressing a nucleic acid construct encoding a CAR and a CCR.

The methods for administering the cell compositions contemplated in particular embodiments includes any method which is effective to result in reintroduction of ex vivo genetically modified immune effector cells that either directly express a CAR and a CCR in the subject or on reintroduction of the genetically modified progenitors of immune effector cells that on introduction into a subject differentiate into mature immune effector cells that express the CAR and CCR. One method comprises transducing peripheral blood T cells ex vivo with a nucleic acid construct contemplated herein and returning the transduced cells into the subject.

L. SEQUENCE LISTING

| | | SEQ |
|---|---|---|
| | | ID |
| Description | Sequence | NO: |
| Human CD8α | MALPVTALLL PLALLLHAAR PSQFRVSPLD RTWNLGETVE | 1 |
| protein | LKCQVLLSNP TSGCSWLFQP RGAAASPTFL LYLSQNKPKA | |
| (Swiss-Prot | AEGLDTORFS GKRLGDTFVL TLSDERRENE GYYFCSALSN | |
| accession | SIMYFSHFVP VFLPAKPTTT PAPRPPTPAP TIASQPLSLR | |
| number P01732) | PEACRPAAGG AVHTRGLDFA CDIYIWAPLA GTCGVLLLSL | |
| | VITLYCNHRN RRRVCKCPRP VVKSGDKPSL SARYV | |
| CD8α hinge | TTTPAPRPPT PAPTIASQPL SLRPEACRPA AGGAVHTRGL | 2 |
| | DFACD | |
| CD8α hinge | TTTPAPRPPT PAPTIASQPL SLRPEASRPA AGGAVHTRGL | 3 |
| (C27S) | DFACD | |
| CD8α hinge | TTTPAPRPPT PAPTIASQPL SLRPEACRPA AGGAVHTRGL | 4 |
| (C44S) | DFASD | |
| CD8α hinge | TTTPAPRPPT PAPTIASQPL SLRPEASRPA AGGAVHTRGL | 5 |
| (C27S; C44S) | DFASD | |
| CD8α TM region | IYIWAPLAGT CGVLLLSLVI T | 6 |
| CD4 hinge | SGQVLLESNI KVLPTWSTPV QP | 7 |
| CD28 hinge | KHLCPSPLFP GPSKP | 8 |
| CD7 hinge | APPRASALPA PPTGSALPDP QTASALPDPP AASALP | 9 |
| CD152 hinge | DTGLYICKVE LMYPPPYYLG IGNGTQIYVI DPEPCPDSD | 10 |
| PD-1 hinge | QIKESLRAEL RVTERRAEVP TAHPSPSPRP AGQFQTLV | 11 |
| IgG1 hinge | EPKSCDKTHT CPPCP | 12 |
| IgG2 hinge | ERKCCVECPP CP | 13 |
| IgG3 hinge | ELKTPLGDTT HTCPRCPEPK SCDTPPPCPR CPEPKSCDTP | 14 |
| | PPCPRCPEPK SCDTPPPCPR CP | |
| IgG4 hinge | ESKYGPPCPS CP | 15 |
| IgG1 hinge- | EPKSCDKTHT CPPCPAPPVA GPSVFLFPPK PKDTLMIART | 16 |
| CH2-CH3 | PEVTCVVVDV SHEDPEVKEN WYVDGVEVHN AKTKPREEQY | |
| | NSTYRVVSVL TVLHQDWLNG KEYKCKVSNK ALPAPIEKTI | |
| | SKAKGQPREP QVYTLPPSRD ELTKNQVSLT CLVKGFYPSD | |
| | IAVEWESNGQ PENNYKTTPP VLDSDGSFFL YSKLTVDKSR | |
| | WQQGNVFSCS VMHEALHNHY TQKSLSLSPG K | |
| IgG1 Hinge- | EPKSCDKTHT CPPCPGQPRE PQVYTLPPSR DELTKNQVSL | 17 |
| CH3-Hinge-M1 | TCLVKGFYPS DIAVEWESNG QPENNYKTTP PVLDSDGSFF | |
| | LYSKLTVDKS RWQQGNVFSC SVMHEALHNH YTQKSLSLSP | |
| | GKGLQLDETC AEAQDGELDG | |
| IgG4 hinge- | ESKYGPPCPS CPAPEFEGGP SVFLFPPKPK DTLMISRTPE | 18 |
| CH2-CH3 | VTCVVVDVSQ EDPEVQFNWY VDGVEVHNAK TKPREEQFQS | |
| | TYRVVSVLTV LHQDWLNGKE YKCKVSNKGL PSSIEKTISK | |
| | AKGQPREPQV YTLPPSQEEM TKNQVSLTCL VKGFYPSDIA | |
| | VEWESNGQPE NNYKTTPPVL DSDGSFFLYS RLTVDKSRWQ | |
| | EGNVFSCSVM HEALHNHYTQ KSLSLSLGK | |
| IgG4 hinge-CH2 | SSESKYGPPC PSCPAPEFEG GPSVFLFPPK PKDTLMISRT | 19 |
| | PEVTCVVVDV SQEDPEVQFN WYVDGVEVHN AKTKPREEQF | |
| | QSTYRVVSVL TVLHQDWLNG KEYKCKVSNK GLPSSIEKTI | |
| | SKAK | |
| PD1 hinge | SGTYLCGAIS LAPKAQIKES LRAELRVTER RAEVPTAHPS | 20 |
| (long) | PSPRPAGQFQ TLV | |

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Activation of T Cell Signaling in the Presence of CCR Antigen Alone

T cells were modified using lentiviral transduction to deliver constructs encoding either CAR molecules or CAR and CCR (CAR/CCR) molecules. The CAR construct expressed an anti-BCMA scFv fused to 4-1BB and CD3z signaling domains (BBz). The CAR+CCR construct expressed the BBz anti-BCMA CAR and an anti-EGFR scFv fused to a CD28 signaling domain CCR, which formed the CCR. The CAR and CCR sequences within the CAR+CCR construct were separated using a 2A ribosomal skipping element.

Modified T cells were then cocultured with the EGFR+HT-1080 tumor cell line, which lacks endogenous BCMA expression. In FIG. 1A, culture supernatants were collected 24 hours later and IFNγ production assessed by Luminex assay. As shown in FIG. 1A, unmodified T cells (UTD) or anti-BCMA CAR T cell failed to secrete IFNγ in the absence of antigen exposure. CAR+CCR T cells, on the other hand, produced detectable amounts of IFNγ despite only being exposed to EGFR.

Similarly, in FIG. 1B, only CAR+CCR T cells were capable of killing EGFR+tumor targets in a dose dependent manner during a standard 4 hour in vitro killing/cytotoxicity assay.

### Example 2

### CCR-Dependent Signaling Reduces Tumor Volume

Immunocompromised NSG mice were implanted subcutaneously with human A549 tumor cells that endogenously express EGFR but lack BCMA expression. Approximately 20 days following implantation, mice were treated with unmodified T cells (UTD), anti-BCMA CAR T cells or anti-BCMA CAR+anti-EGFR CCR T cells and tumor volume was assessed. As shown in FIG. 2, neither untransduced nor anti-BCMA CAR T cells were capable of controlling tumor growth and ultimately those mice succumbed to tumorigenesis. Alternatively, mice treated with anti-BCMA CAR+anti-EGFR CCR T cells saw complete tumor regressions for the duration of the study.

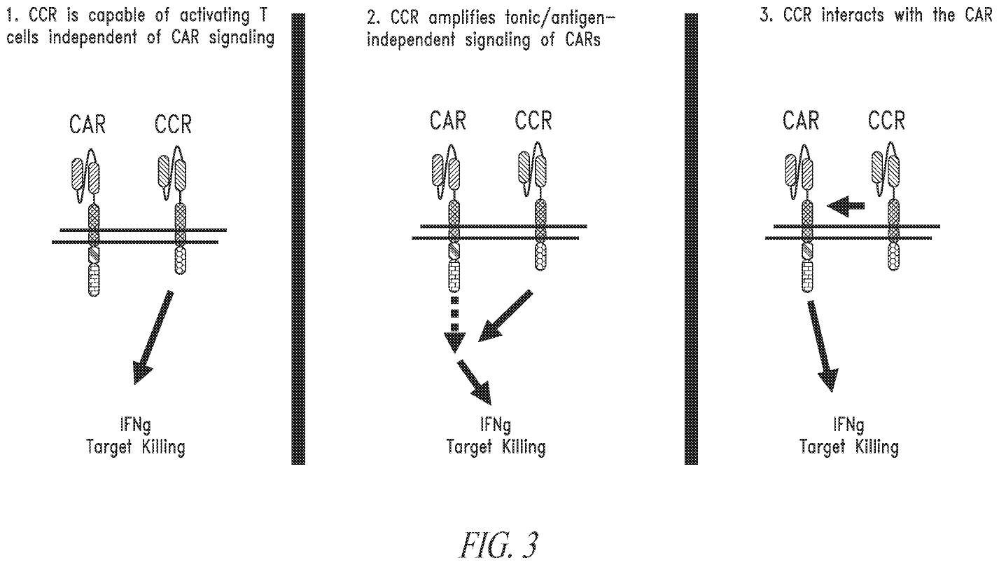

Without wishing to be bound by any particular theory, FIG. 3 depicts three illustrative and non-limiting models explaining how T cells endowed with a CAR and a CCR are capable of reacting to tumor target cells that express the CCR antigen while lacking the CAR antigen. The CCR is capable of binding the CCR antigen and subsequently transducing a signal independent of the CAR molecule (FIG. 3, left panel). Antigen independent basal CAR signaling through . CD3z is enhanced by the CCR co-stimulatory signal to a level that is now detectable by standard in vitro experiments and that manifests as tumor control in vivo (FIG. 3, left panel). A molecular interaction between the CAR and CCR molecules exists such that engagement of the CCR via the CCR antigen initiates signal transduction and triggers T cell activation.

### Example 3

### CAR and CCR Hinge Mutations Modulate CAR T Cell Signaling

An experiment was designed to test whether any one of the four cysteines in the CD8a hinge regions of the CAR and the CCR (two in the CAR and two in the CCR) were responsible for a molecular interaction between the two different proteins. Six different CAR+CCR constructs that varied in their usage of cysteines and serines at positions 289 and 306 of the CD8a hinge (correspond to positions 27 and 44 of SEQ ID NO: 2) of either the CAR or the CCR were generated and transduced into T cells. C1 is the parental CAR+CCR construct that utilizes cysteines in all four positions. C2 contains two serines in the CAR and two cysteines in the CCR. C3 contains two cysteines in the CAR and two serines in the CCR. C4 contains a serine at position 27 of SEQ ID NO: 2 and a cysteine at position 44 of SEQ ID NO: 2 of the CAR; and a cysteine at position 27 of SEQ ID NO: 2 and a serine at position 44 of SEQ ID NO: 2 of the CCR. C5 contains a cysteine at position 27 of SEQ ID NO: 2 and a serine at position 44 of SEQ ID NO: 2 of the CAR and serine at position 27 of SEQ ID NO: 2 and a cysteine at position 44 of SEQ ID NO: 2 of the CCR. C6 utilizes serines in all four positions.

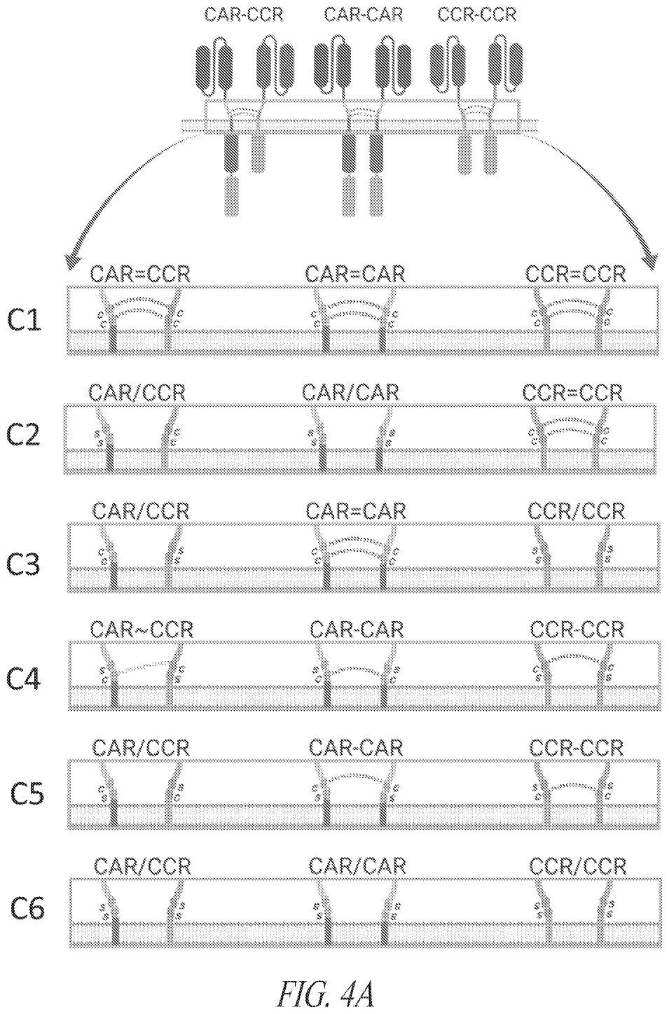

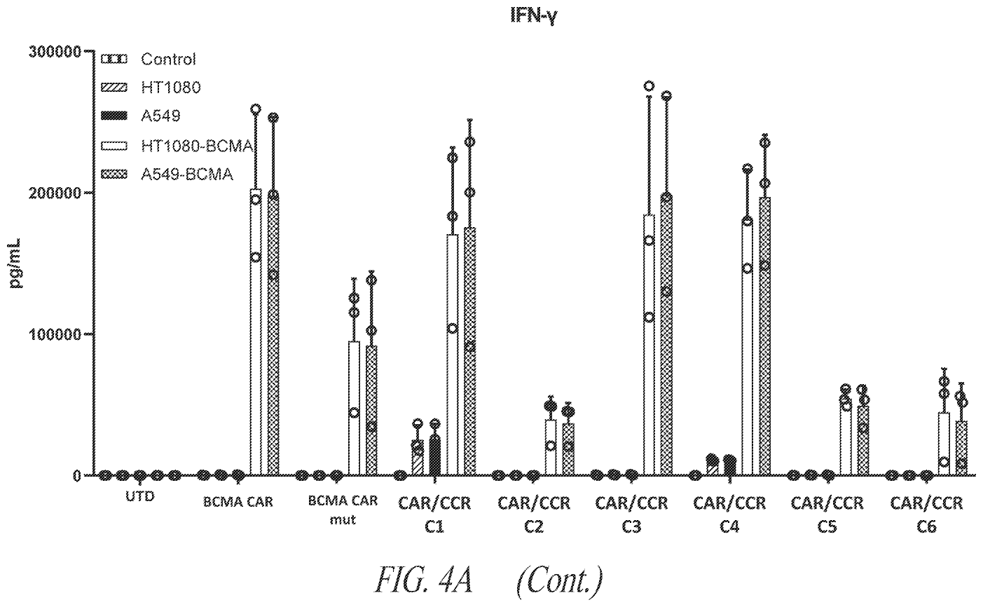

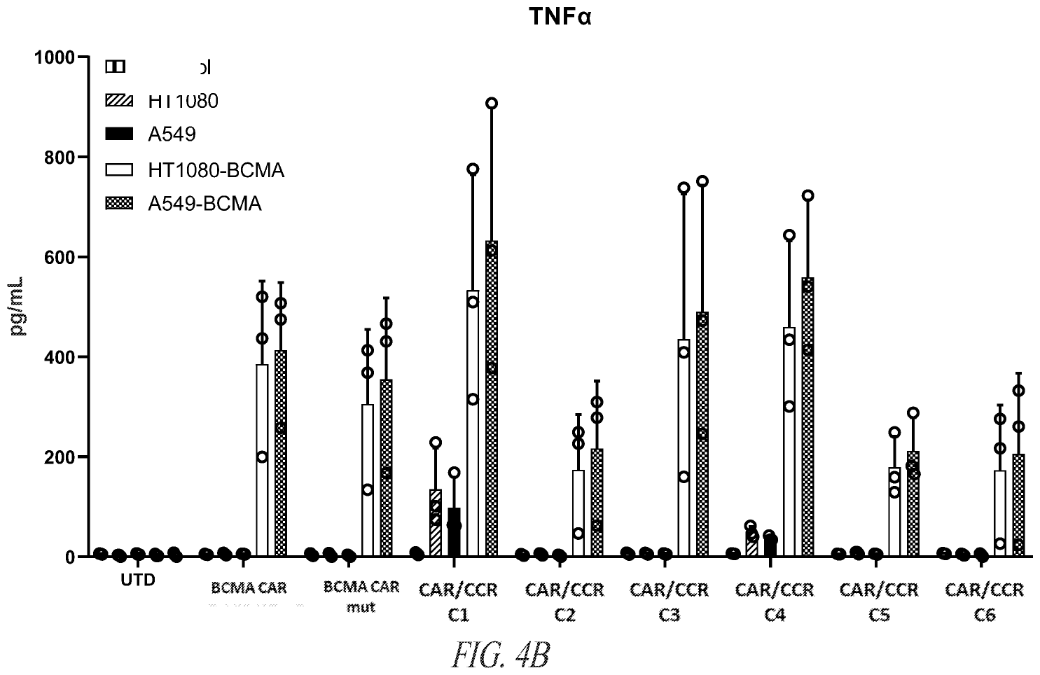

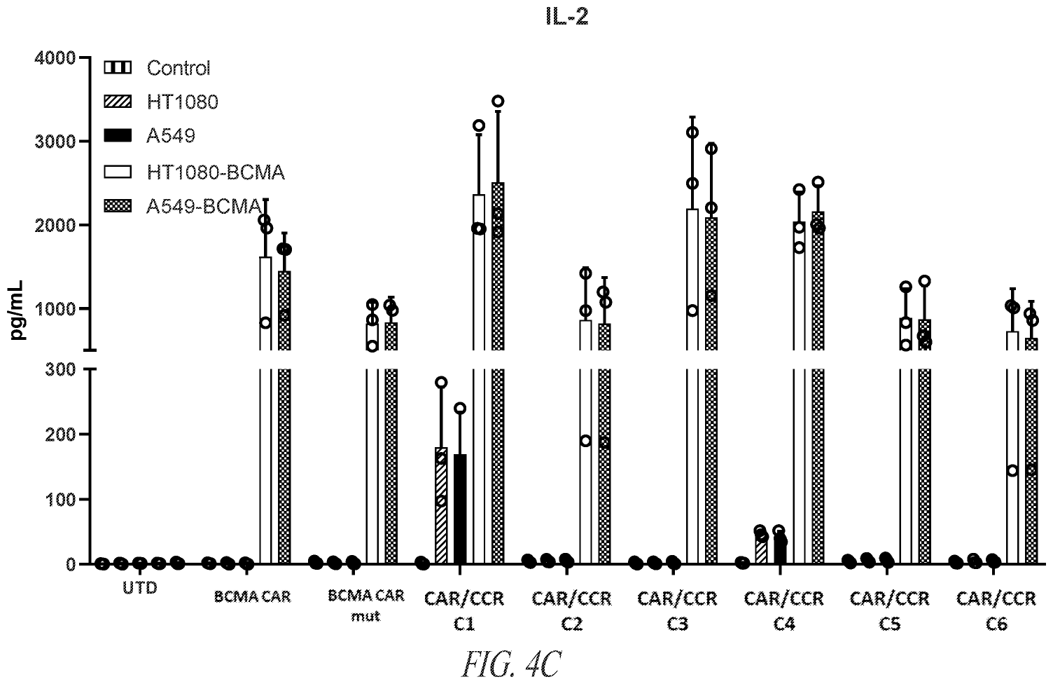

T cells were modified with the six constructs described above as well as two controls: an anti-BCMA BBz CAR and a variant of the anti-BCMA CAR that replaces the cysteines at positions 27 and 44 of SEQ ID NO: 2 with serines. Each T cell condition was then co-cultured with a variety of tumor cells that either express EGFR only (A549 and HT-1080), or tumor cells that express both BCMA and EGFR (A549.BCMA and HT-1080.BCMA). IFNγ production was assessed 24 hours later. As shown in FIGS. 4A-4C, control anti-BCMA CAR T cells only produced cytokine when cultured in the presence of BCMA expressing tumor cells. The introduction of serines at position 27 and 44 of SEQ ID NO: 2 in the anti-BCMA CAR resulted in reduced IFNγ production when cultured with BCMA expressing tumor cells. T cells engineered to express the parental CAR+CCR construct (C1) responded to tumor cells that either express BCMA and EGFR, or EGFR alone. None of the other CAR+CCR variants used in these experiments were able to retain full reactivity to tumor cells that only express EGFR. Interestingly, construct C4 was the only variant to exhibit partial responsiveness to EGFR expressing tumor cells. Taken together these data demonstrate the importance of hinge cysteines in mediating CAR/CCR molecular interactions.

### Example 4

### Design and Expression of CAR and CCR Fusion Polypeptides

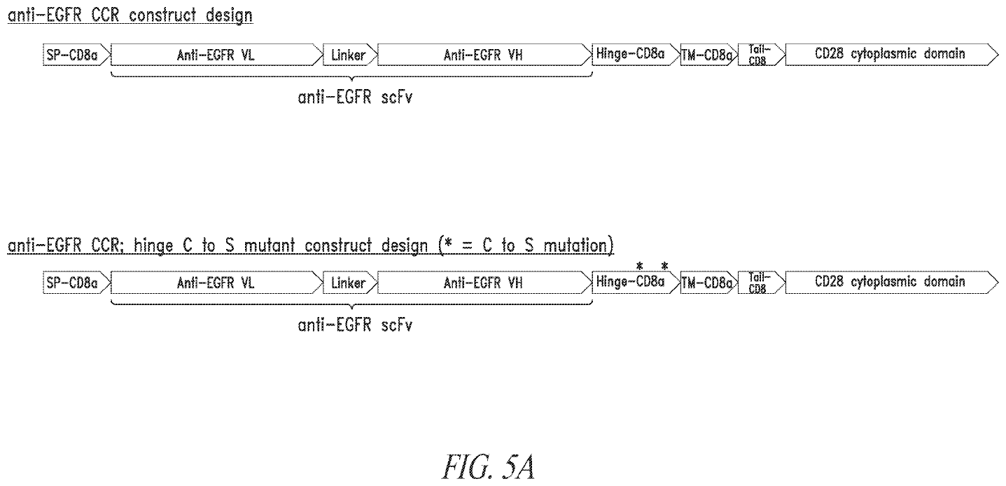

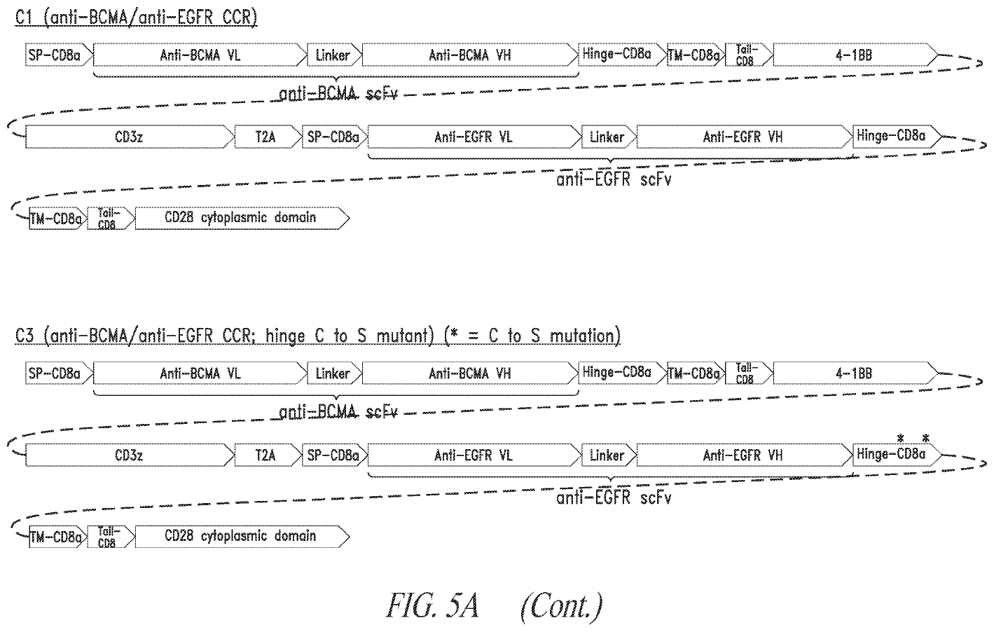

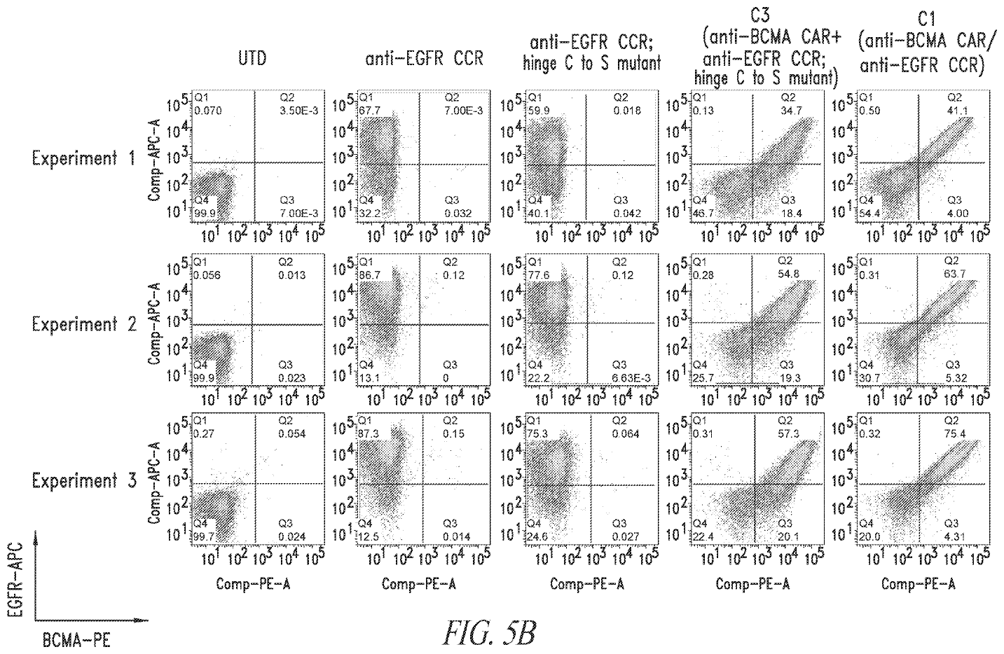

FIG. 5A depicts the modular protein domains used to construct the anti-EGFR CCR, the anti-EGFR CCR containing the two cysteine to serine mutations and the corresponding CAR+CCR constructs (C1 and C3, respectively). C3 is a construct encoding an anti-BCMA CAR and an anti-EGFR CCR with serines in place of cysteines within the CD8a hinge corresponding to positions 27 and 44 of SEQ ID NO: 2. C1 is the parental CAR+CCR construct with all four cysteines present in the CAR and CCR.

Each of these constructs was transduced into T cells from 3 different donors and flow cytometry was used to assess cell surface expression of both the CAR and the CCR compared to the UTD control that does not express either CAR or CCR. As demonstrated in FIG. 5B, the presence of serines at positions 27 and 44 of SEQ ID NO: 2 do not impact the overall transduction efficiency of the CCR only constructs. Similarly, T cells transduced with the C1 or C3 constructs result in the expression of both the CAR and CCR on the cell surface. There is no impact on transduction efficiency despite the presence of serines at both positions of the CCR.

### Example 5

### CCR Hinge Double Mutation Reduces T Cell Activation in the Absence of CAR Antigen

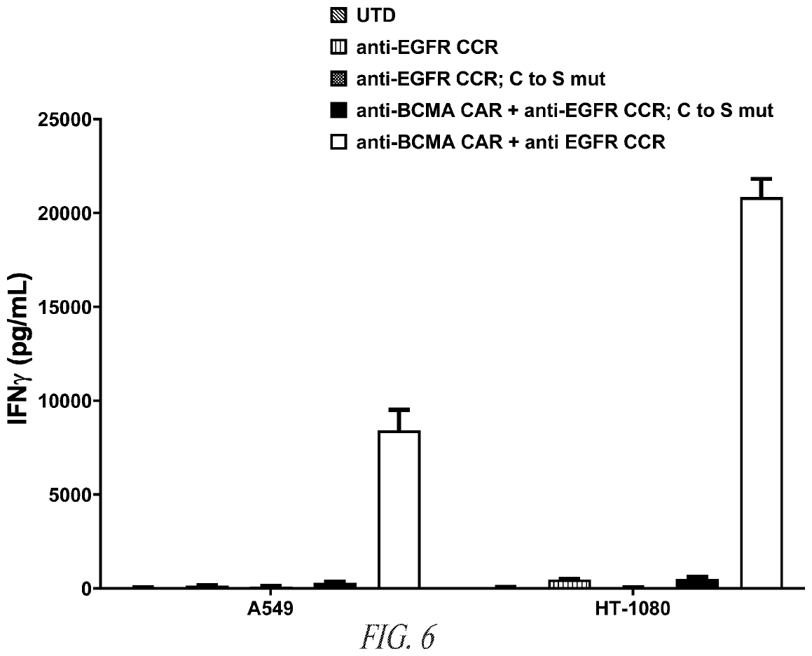

T cells transduced with the constructs described in Example 4 were co-cultured with A549 or HT-1080 tumor cells that only express the antigen against the CCR, EGFR and assessed for IFNγ production 24 hours later. FIG. 6 demonstrates the responsiveness of CAR+CCR T cells against tumor cells that express EGFR but lack BCMA, suggesting that these T cells targeted not just BCMA+ tumors but also tumors that only express EGFR. Upon introduction of serines at positions 27 and 44 of SEQ ID NO: 2 of the CCR, this reactivity is reduced to levels consistent with negative controls, indicating cysteines within the CCR hinge are important for mediating reactivity to EGFR single positive tumor cells.

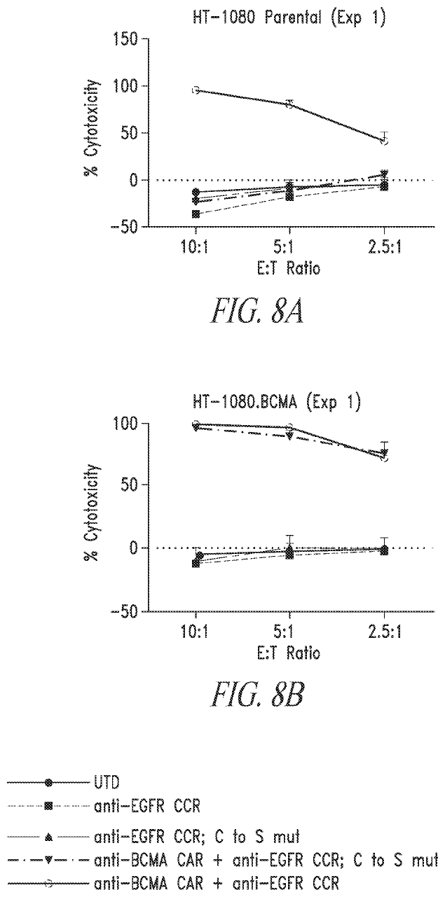

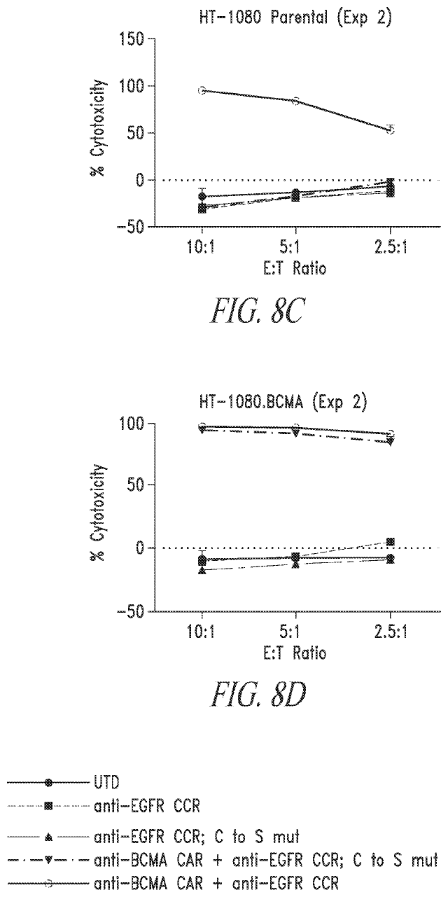

These results were further corroborated by the data in FIGS. 7A-7D and 8A-8D, which demonstrates that CAR+CCR T cells that target BCMA and EGFR increased cytokine release and killing of BCMA+ tumor cells regardless of their cysteine usage. However, only cells that contain their natural cysteines within the hinge domain of the CCR are capable of eliminating EGFR single positive tumor cells, whereas those T cells that contain serines in place of cysteines in the hinge of the CCR are unable to kill those tumor cells (FIGS. 8A-8D) in the absence of BCMA.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1-130. (canceled)

131. A fusion polypeptide comprising:
a) a chimeric antigen receptor (CAR) comprising a first hinge region;
b) a polypeptide cleavage signal; and
c) a chimeric co-stimulatory receptor (CCR) comprising a second hinge region comprising one or more cysteines substituted with another amino acid.

132. The fusion polypeptide of claim 131, wherein the one or more cysteine substitutions within the second hinge region (i) reduces CCR antigen-mediated stimulation of T cell signaling in the absence of CAR antigen, compared to a CCR comprising a second hinge region without cysteine substitutions and/or (ii) reduces CAR/CCR association in the absence of CAR antigen, compared to a CCR comprising a second hinge region without cysteine substitutions.

133. The fusion polypeptide of claim 131, wherein the first hinge region comprises one or more cysteines substituted with a different amino acid and wherein the first hinge region (i) reduces CCR antigen-mediated stimulation of T cell signaling in the absence of CAR antigen compared to a CAR comprising a first hinge region without cysteine substitutions and/or (ii) reduces CAR/CCR association in the absence of CAR antigen compared to a CAR comprising a first hinge region without cysteine substitutions.

134. The fusion polypeptide of claim 131, wherein the first and second hinge regions are CD8α hinge regions or a functional fragment thereof.

135. The fusion polypeptide of claim 131, wherein the first or second CD8α hinge region comprises an amino acid substitution at position 27 of SEQ ID NO: 2.

136. The fusion polypeptide of claim 131, wherein the first or second CD8α hinge region comprises an amino acid substitution at position 44 of SEQ ID NO: 2.

137. The fusion polypeptide of claim 131, wherein the second CD8α hinge region comprises amino acid substitutions at positions 27 and 44 of SEQ ID NO: 2.

138. The fusion polypeptide of claim 131, wherein the first CD8α hinge region comprises an amino acid substitution at position 27 of SEQ ID NO: 2, and the second CD8α hinge region comprises an amino acid substitution at position 44 of SEQ ID NO: 2.

139. The fusion polypeptide of claim 131, wherein the first or second hinge region comprises an amino acid sequence set forth in SEQ ID NO: 3.

140. The fusion polypeptide of claim 131, wherein the second hinge region comprises an amino acid sequence as set forth in SEQ ID NO: 4.

141. The fusion polypeptide of claim 131, wherein the second hinge region comprises an amino acid sequence as set forth in SEQ ID NO: 5.

142. A polynucleotide encoding the fusion polypeptide of claim 131.

143. A vector comprising the polynucleotide of claim 142.

144. The vector of claim 143, wherein the vector is a lentiviral vector.

145. A cell that expresses (i) the fusion polypeptide or (ii) the CAR and CCR of claim 131.

146. The cell of claim 145, wherein the cell is (i) an immune effector cell, (ii) a T-cell, (iii) a CD3+, CD4+, and/or CD8+ cell, (iv) a cytotoxic T lymphocytes (CTLs), a tumor infiltrating lymphocytes (TILs), or a helper T cell, (v) a natural killer (NK) or natural killer T (NKT) cell, or (vi) a macrophage.

147. A composition comprising the cell of claim 145.

148. A method of treating a cancer in a subject in need thereof, comprising administering to the subject a therapeutically effective amount of the composition of claim 147.

149. A method of reducing CCR antigen-mediated stimulation of T cell signaling in the absence of CAR antigen, compared to a CCR comprising a hinge domain without substitutions, the method comprising:
a) obtaining CAR and CCR polypeptides each having a hinge domain;
b) substituting one or more cysteine residues within the CCR hinge domain for another residue, thereby producing a modified CCR; and
c) expressing the CAR and modified CCR in a cell.

150. The method of claim 149, wherein the CAR hinge region comprises one or more cysteines substituted with a different amino acid.
